(19) United States
(12) Patent Application Publication
(10) Pub. No.: US 2017/0172438 A1
(43) Pub. Date: Jun. 22, 2017

(54) SYSTEMS AND METHODS FOR INJECTABLE DEVICES

(71) Applicant: President and Fellows of Harvard College, Cambridge, MA (US)

(72) Inventors: Charles M. LIEBER, Lexington, MA (US); Jia LIU, Palo Alto, CA (US); Zengguang CHENG, Summertown, Oxford (GB); Guosong HONG, Somerville, MA (US); Tian-Ming FU, Somerville, MA (US); Tao ZHOU, Somerville, MA (US)

(21) Appl. No.: 15/301,792

(22) PCT Filed: Apr. 3, 2015
(86) PCT No.: PCT/US2015/024252

§ 371 Date: Oct. 4, 2016

Related U.S. Application Data
(60) Provisional application No. 61/975,601, filed on Apr. 4, 2014.

(51) Int. Cl. A61B 5/04; A61N 1/05

(52) U.S. Cl. CPC A61B 5/04001; A61N 1/05

(57) ABSTRACT

The present invention generally relates to nanoscale wires and/or injectable devices. In some embodiments, the present invention is directed to electronic devices that can be injected or inserted into soft matter, such as biological tissue or polymeric matrixes. For example, the device may be passed through a syringe or a needle. In some cases, the device may comprise one or more nanoscale wires. Other components, such as fluids or cells, may also be injected or inserted. In addition, in some cases, the device, after insertion or injection, may be connected to an external electrical circuit, e.g., to a computer. Other embodiments are generally directed to systems and methods of making, using, or promoting such devices, kits involving such devices, and the like.

### RELATED APPLICATIONS

This application claims the benefit of U.S. Provisional Patent Application Ser. No. 61/975,601, filed Apr. 4, 2014, entitled “Systems and Methods for Injectable Devices,” incorporated herein by reference in its entirety.

### GOVERNMENT FUNDING

This invention was made with government support under Grant No. 8DP1GM105379-05 awarded by the National Institutes of Health, and under Grant No. N00244-09-1-0078 awarded by the Department of Defense. The government has certain rights in the invention.

### FIELD

The present invention generally relates to nanoscale wires and/or injectable devices.

### BACKGROUND

Recent efforts in coupling electronics and tissues have focused on flexible, stretchable planar arrays that conform to tissue surfaces, or implantable microfabricated probes. These approaches have been limited in merging electronics with tissues while minimizing tissue disruption, because the support structures and electronic detectors are generally of a much larger scale than the extracellular matrix and the cells. Furthermore, planar arrays only probe the tissue near the device plane surface and cannot be used to study the internal 3-dimensional structure of the tissue. For example, probes using nanowire field-effect transistors have shown that electronic devices with nanoscopic features can be used to detect extra- and intracellular potentials from single cells, but are limited to only surface recording from 3-dimensional tissues and organs.

### SUMMARY

The present invention generally relates to nanoscale wires and/or injectable devices. The subject matter of the present invention involves, in some cases, interrelated products, alternative solutions to a particular problem, and/or a plurality of different uses of one or more systems and/or articles.

In one aspect, the present invention is generally directed to passing a device comprising one or more nanoscale wires through a tube. In another, the present invention is generally directed to passing a device comprising one or more nanoscale wires through an opening of a tube. In another aspect, the present invention is generally directed to passing a device comprising one or more nanoscale wires through an injection device. In yet another aspect, the present invention is generally directed to injecting a device comprising one or more nanoscale wires into a subject. In still another aspect, the present invention is generally directed to injecting a device comprising one or more nanoscale wires into soft matter. In some cases, at least one of the nanoscale wires is a silicon nanowire.

In one aspect, the present invention is generally directed to a tube comprising a device comprising one or more nanoscale wires. The present invention, in another aspect, is generally directed to a needle comprising a device comprising one or more nanoscale wires. The present invention, in yet another aspect, is generally directed to a syringe comprising a device comprising one or more nanoscale wires. In some cases, at least one of the nanoscale wires is a silicon nanowire.

In another aspect, the present invention is generally directed to a tube inserted into a subject, wherein the tube comprises a device comprising one or more nanoscale wires. In another aspect, the present invention is generally directed to a needle inserted into a subject, wherein the needle comprises a device comprising one or more nanoscale wires. In another aspect, the present invention is generally directed to a syringe inserted into a subject, wherein the syringe comprises a device comprising one or more nanoscale wires. In some cases, at least one of the nanoscale wires is a silicon nanowire.

In another aspect, the present invention is generally directed to a tube inserted into soft matter, wherein the tube comprises a device comprising one or more nanoscale wires. In another aspect, the present invention is generally directed to a needle inserted into soft matter, wherein the needle comprises a device comprising one or more nanoscale wires. In another aspect, the present invention is generally directed to a syringe inserted into soft matter, wherein the syringe comprises a device comprising one or more nanoscale wires. In some cases, at least one of the nanoscale wires is a silicon nanowire.

In another aspect, the present invention encompasses methods of making one or more of the embodiments described herein, for example, a device comprising one or more nanoscale wires. The device may be injectable in some cases. In still another aspect, the present invention encompasses methods of using one or more of the embodiments described herein, for example, a device comprising one or more nanoscale wires. The device may be injectable in some cases.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1G illustrate certain devices in accordance with various embodiments of the invention;

FIGS. 2A-2E show injection of certain devices, according to some embodiments of the invention;

FIGS. 3A-3C show analysis of an injection process, according to one embodiment of the invention;

FIGS. 4A-4D show injectable electronics devices, in accordance with some embodiments of the invention;

FIGS. 5A-5J illustrate injectable electronics devices for certain biological systems, in accordance with some embodiments of the invention;

FIGS. 6A-6E shows optical images of certain device structures, in certain embodiments of the invention;

FIGS. 7A-7B illustrate device meshes of nanoscale wires, in some embodiments of the invention;

FIGS. 8A-8D illustrate injection of devices, in accordance with one set of embodiments of the invention;

FIGS. 9A-9D illustrate bonding in devices, in certain embodiments of the invention; FIGS. 10A-10F illustrate shows electronic devices within a needle, in some embodiments of the invention;

FIGS. 11A-11C shows a rolled mesh device, in one embodiment;

FIGS. 12A-12D illustrate injection of a device into soft matter, in another embodiment of the invention;

FIGS. 13A-13D illustrate injection of a device in vivo, in yet another embodiment of the invention;

FIGS. 14A-14D illustrate an interface between a device and tissue, in still other embodiments of the invention; aH4

FIG. 15 schematically illustrates a cross-section of one embodiment of the invention;

FIGS. 16A-16J show syringe injectable electronics in accordance with certain embodiments of the invention;

FIGS. 17A-17E show mesh electronics structures in various embodiments of the invention;

FIGS. 18A-18G show injection of mesh electronics in some embodiments of the invention; and

FIGS. 19A-19L show syringe injectable electronics in biological systems, in accordance with certain embodiments of the invention.

### DETAILED DESCRIPTION

The present invention generally relates to nanoscale wires and/or injectable devices. In some embodiments, the present invention is directed to electronic devices that can be injected or inserted into soft matter, such as biological tissue or polymeric matrixes. For example, the device may be passed through a syringe or a needle. In some cases, the device may comprise one or more nanoscale wires. Other components, such as fluids or cells, may also be injected or inserted. In addition, in some cases, the device, after insertion or injection, may be connected to an external electrical circuit, e.g., to a computer. Other embodiments are generally directed to systems and methods of making, using, or promoting such devices, kits involving such devices, and the like.

One aspect of the present invention is generally directed to a device for insertion or injection into a tissue (e.g., biological tissue), or other matter, including soft matter. The tissue may be in vitro or in vivo (i.e., the device may be injected into a living subject). In some cases, soft matter is matter that exhibits some viscoelasticity, e.g., the matter can undergo deformation, and may exhibit viscous and/or elastic characteristics while undergoing deformation. Examples of soft matter include, but are not limited to, polymers, gels, or other materials having viscoelastic properties. The device can be fully or partially inserted into the tissue or other matter. The device may be used to determine a property of the tissue or other matter, and/or provide an electrical signal to the tissue or other matter. This may be achieved using one or more nanoscale wires on the device. In some cases, at least one of the nanoscale wires is a silicon nanowire. In certain embodiments, a device comprising nanoscale wires may be inserted into an electrically-active tissue, such as the heart or the brain, and the nanoscale wires may be used to determine electrical properties of the tissue, e.g., action potentials or other electrical activity. In some cases, the device is relatively porous to allow cells, etc. to grow or migrate into the device, for example, neurons may grow into the device. This may be useful, for example, for long-term applications, for example, where the device is to be inserted and used for days, weeks, months, or years within the tissue. For example, neurons or cardiac cells may be able to grow around and/or into the device while it is inserted into the brain or the heart, e.g., over extended periods of time.

In some embodiments, a device may be formed from one or more polymeric constructs and/or metal leads. In some cases, the device is relatively small and may include components such as nanoscale wires. The device may also be flexible and/or have a relatively open structure, e.g., an open porosity of at least about 30%, or other porosities as discussed herein. For instance, the device may be formed from a plurality of nanoscale wires, connected by polymeric constructs and/or metal leads, forming a relatively large or open network, which can then be rolled to form a cylindrical or other 3-dimensional structure that is to be inserted into a subject. In some cases, the nanoscale wires may be distributed about the device, e.g., in three dimensions, thereby allowing determining properties and/or stimulation of a tissue, etc. in three-dimensions. The device can also be connected to an external electrical system, e.g., to facilitate use of the device. Polymeric constructs, metal leads, nanoscale wires, the structure of the device, and various properties of the devices are all discussed in additional detail below.

For instance, in certain aspects, a device as discussed herein may be positioned in a tube, such as a metal tube. The device may be shaped such that it is cylindrical or curved, and/or the device may be compressed to fit inside the tube, although the device may be able to expand after exiting the tube, e.g., as discussed herein. The tube may be formed out of any suitable material. For instance, the tube may comprise stainless steel. The tube may also be other materials in other embodiments. For example, the tube may be plastic, or the tube may be glass. The tube may be a needle or form part of a syringe, or the tube may be form part of an injector device, such as a microinjector. In some cases, the tube is cylindrical, although the tube may be noncylindrical in other cases. For instance, the tube may be tapered in some embodiments. In some cases, the tube is hollow. In some cases, the tube has a circular cross-section. However, in other cases, the tube may not have a circular cross-section. For example, the tube may have a square or rectangular cross-section, or the tube may have an open cross-section, e.g., having a “U”-shaped cross section. The tube may have any suitable inner diameter. For instance, the tube may have an inner diameter of less than about 1 mm, less than about 800 micrometers, less than about 600 micrometers, less than about 500 micrometers, less than about 400 micrometers, less than about 300 micrometers, less than about 200 micrometers, less than about 100 micrometers, less than about 80 micrometers, less than about 60 micrometers, less than about 50 micrometers, etc.

The device may pass through the tube using any suitable method. The device may fully pass through the tube, or the device may only partially pass through the tube such that a portion of the device remains within the tube. For instance, the device may be fully or partially expelled or urged from the tube using suitable forces, pressures, mechanisms, or apparatuses. For instance, in one set of embodiments, the device may be expelled using a microinjection device. In another embodiment, the device may be manually expelled, e.g., by pushing the plunger of a syringe. In some cases, fluids (liquids or gases) may be added to the tube to expel the device. For instance, water, saline, or air may be added to the tube to cause the device to be expelled therefrom. In some cases, for example, a pump or other fluid source (e.g., a spigot or a tank) may be used to introduce fluid into the tube to expel the device. For instance, a pump may pump fluid into the tube (or through tubing or other fluidic channels) into the tube to cause the device to be expelled therefrom (e.g., partially or fully). The device may be injected at a controlled rate and/or with controllable position, for example, by controlling the pressure or flow rate of fluid from the pump. In some cases, the tube may be inserted into a target such that the device is expelled directly into the target. For example, the tube may be inserted into a subject, e.g., into the tissue of a subject, such as those described herein. In another embodiment, the tube may be inserted into soft matter. For instance, the tube may be inserted into a polymer or a gel. Thus, the device may be expelled from the tube such that the device at least partially penetrates into the target.

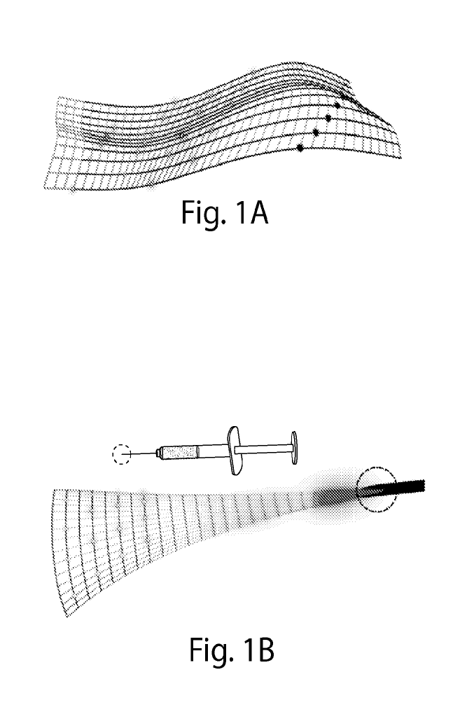

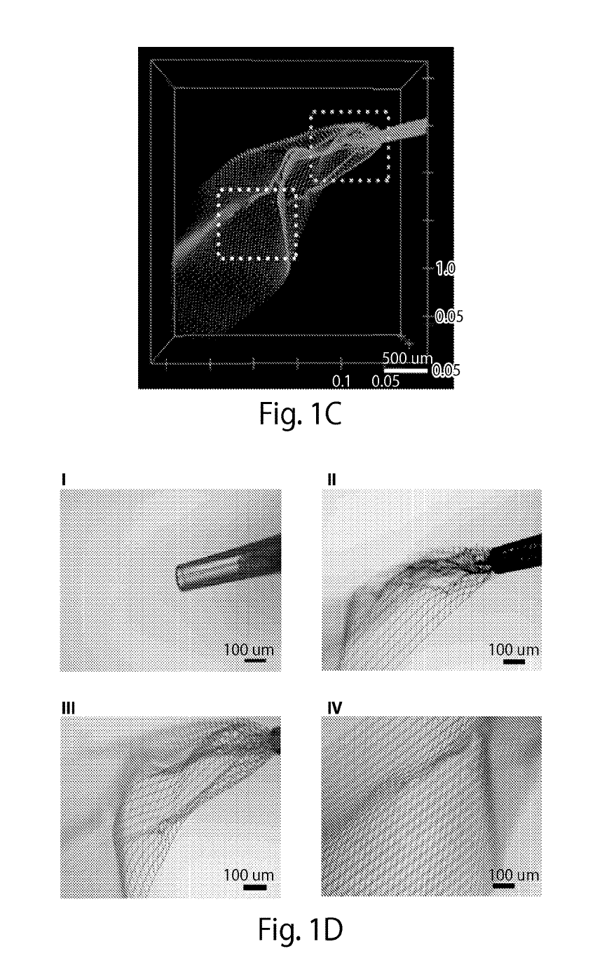

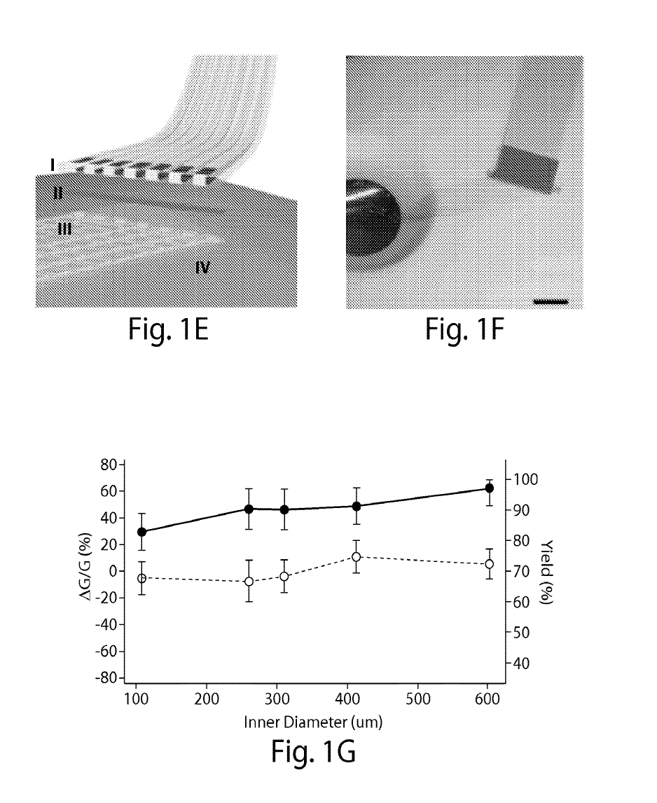

As mentioned, in some cases, the device, when inserted into the tube, is constrained or compressed in some fashion such that, upon expulsion (fully or partially), the device is able to at least partially expand. As a non-limiting example, the device may be a network that is rolled to form a cylinder; upon expulsion, the device is able to at least partially unroll and expand, e.g., as is shown in FIG. 1B. In some cases, the device is able to spontaneously expand, e.g., upon exiting the tube. The expansion may occur rapidly, or on longer time scales. As another example, the device may unfold, or the device may uncompress, upon exiting a tube. The device may expand to reach its original shape. In some cases, the device may substantially return to its original shape after about 24 hours, after about 48 hours, or after about 72 hours. In certain embodiments, it may take longer for the device to substantially return to its original shape, e.g., after 1 week, after 2 weeks, after 3 weeks, after 4 weeks, after 5 weeks, after 6 weeks, etc. In some cases, however, the device may not necessarily return to its original shape, e.g., inherently, and/or due to the matter that the device was injected or inserted into. For example, the presence of tissue (or other matter) may prevent the device from fully expanding back to its original shape after insertion.

In some aspects, other materials may also be present within the tube, e.g., in addition to the device. For example, in one set of embodiments, a gas or a liquid may be present within the tube. For instance, the tube may contain a liquid to facilitate expulsion of the device, or a liquid to assist in movement of the device out of the tube, or into the target. For instance, the tube may include a liquid such as saline, which can be injected into a subject, e.g., along with the device. In addition, in some cases, the fluid may also contain one or more cells, which may be inserted or injected into a target along with the device. If the target is a subject or biological tissue, the cells may be autologous, heterologous, or homologous to the tissue or to the subject.

In certain aspects, the device may comprise one or more electrical networks comprising nanoscale wires and conductive pathways in electrical communication with the nanoscale wires. In some cases, at least some of the conductive pathways may also provide mechanical strength to the device, and/or there may be polymeric or metal constructs that are used to provide mechanical strength to the device. The device may be planar or substantially define a plane, or the device may be non-planar or curved (i.e., a surface that can be characterized as having a finite radius of curvature). The device may also be flexible in some cases, e.g., the device may be able to bend or flex. For example, a device may be bent or distorted by a volumetric displacement of at least about 5%, about 10%, or about 20% (relative to the undisturbed volume), without causing cracks and/or breakage within the device. For example, in some cases, the device can be distorted such that about 5%, about 10%, or about 20% of the mass of the device has been moved outside the original surface perimeter of the device, without causing failure of the device (e.g., by breaking or cracking of the device, disconnection of portions of the electrical network, etc.). In some cases, the device may be bent or flexed as described above by an ordinary human being without the use of tools, machines, mechanical device, excessive force, or the like. A flexible device may be more biocompatible due to its flexibility, and the device may be treated as previously discussed to facilitate its insertion into a tissue.

In addition, the device may be non-planar in some cases, e.g., curved as previously discussed. For example, the device may be substantially U-shaped or cylindrical, and/or have a shape and/or size that is similar to a hypodermic needle. In some embodiments, the device may be generally cylindrical with a maximum outer diameter of no more than about 5 mm, no more than about 4 mm, no more than about 3 mm, no more than about 2 mm, no more than about 1 mm, no more than about 0.9 mm, no more than about 0.8 mm, no more than about 0.7 mm, no more than about 0.6 mm, no more than about 0.5 mm, no more than about 0.4 mm, no more than about 0.3 mm, or no more than about 0.2 mm. Accordingly, in some embodiments, the device may be able to placed into a tube, e.g., of a needle or a syringe. As discussed herein, the device can then be inserted or injected out of the tube upon application of suitable forces and/or pressures, for instance, such that the device can be inserted or injected into other matter. For instance, the device may be injected into the tissue of a subject, or into a gel.

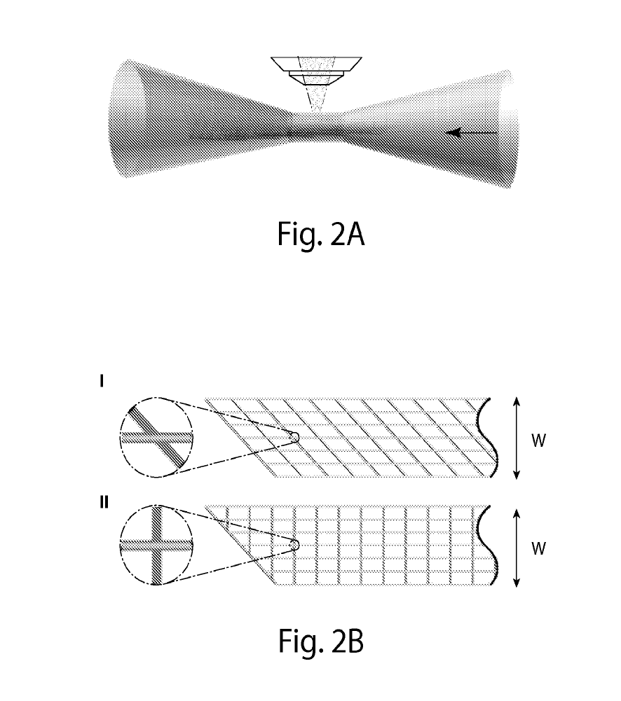

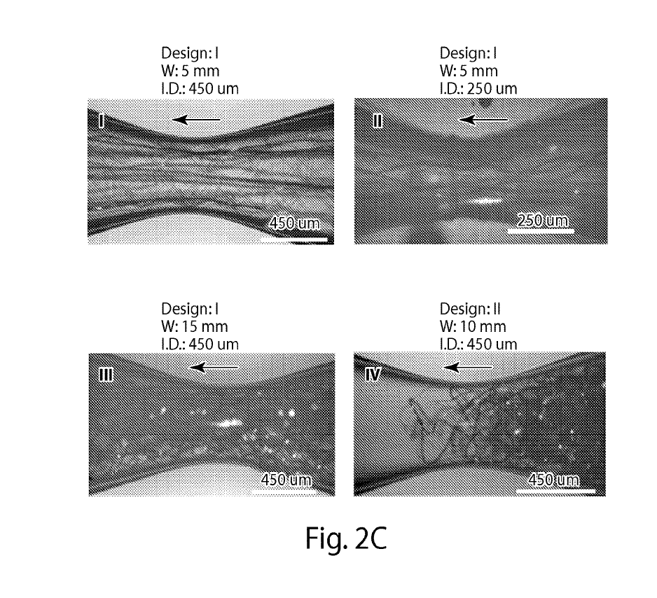

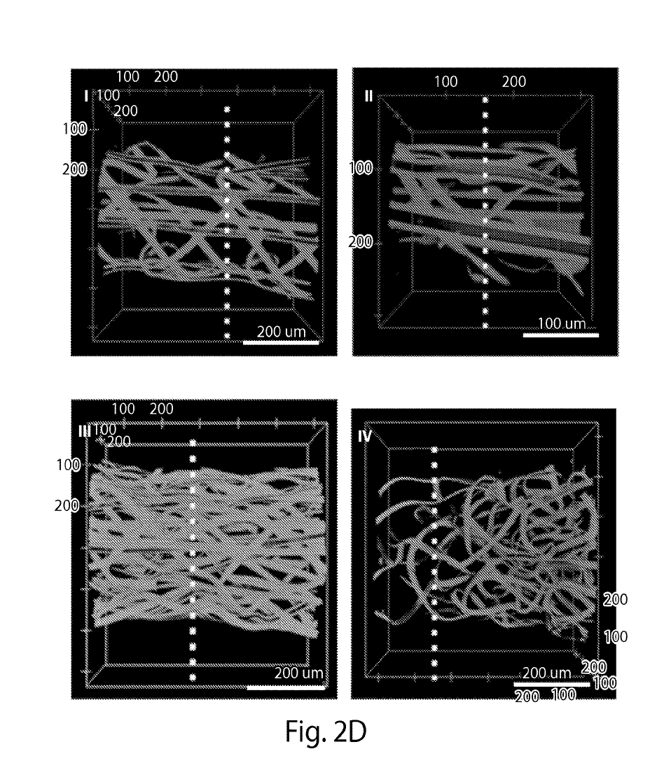

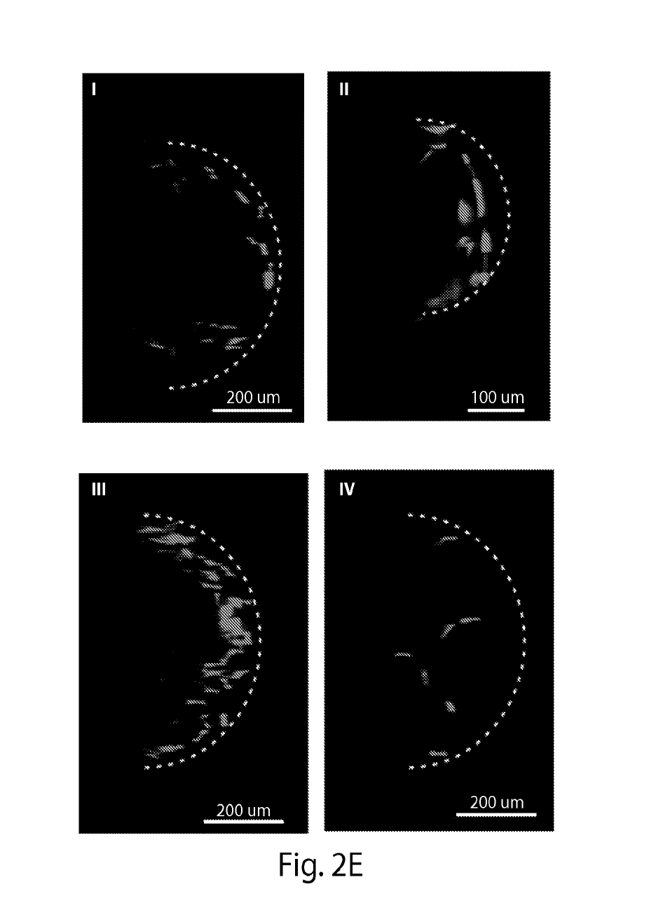

In one aspect, the device may comprise a periodic structure comprising nanoscale wires. For example, the device may comprise a mesh or other two-dimensional array of nanoscale wires and conductive pathways, such as is shown in FIG. 2B. The mesh may include a first set of conductive pathways, generally parallel to each other, and a second set of conductive pathways, generally parallel to each other. The first set and the second set may be orthogonal to each other (e.g., FIG. 2B, II), or they may cross at any suitable angle (e.g., FIG. 2B, I). For instance, the sets may cross at a 30° angle, a 45° angle, or a 60° angle, or any other suitable angle. Mesh structures of the device may be particularly useful in certain embodiments. For instance, in a mesh structure, due to the physical connections, it may be easier for the structure to maintain its topological configuration, e.g., of the nanoscale wires relative to each other. In addition, it may be more difficult for the structure to become adversely tangled. If a periodic structure is used, the period may be of any suitable length. For example, the length of a unit cell within the periodic structure may be less than about less than about 500 micrometers, less than about 400 micrometers, less than about 300 micrometers, less than about 200 micrometers, less than about 100 micrometers, less than about 80 micrometers, less than about 60 micrometers, less than about 50 micrometers, etc.

In certain aspects, the device may contain one or more polymeric constructs. The polymeric constructs typically comprise one or more polymers, e.g., photoresists, biocompatible polymers, biodegradable polymers, etc., and optionally may contain other materials, for example, metal leads or other conductive pathway materials. The polymeric constructs may be separately formed then assembled into the device, and/or the polymeric constructs may be integrally formed as part of the device, for example, by forming or manipulating (e.g. folding, rolling, etc.) the polymeric constructs into a 3-dimensional structure that defines the device.

In one set of embodiments, some or all of the polymeric constructs have the form of fibers or ribbons. For example, the polymeric constructs may have one dimension that is substantially longer than the other dimensions of the polymeric construct. The fibers can in some cases be joined together to form a network or mesh of fibers. For example, a device may contain a plurality of fibers that are orthogonally arranged to form a regular network of polymeric constructs. However, the polymeric constructs need not be regularly arranged. The polymer constructs may have the form of fibers or other shapes. In general, any shape or dimension of polymeric construct may be used to form a device.

In one set of embodiments, some or all of the polymeric constructs have a smallest dimension or a largest cross-sectional dimension of less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. A polymeric construct may also have any suitable cross-sectional shape, e.g., circular, square, rectangular, polygonal, elliptical, regular, irregular, etc. Examples of methods of forming polymeric constructs, e.g., by lithographic or other techniques, are discussed below.

In one set of embodiment, the polymeric constructs can be arranged such that the device is relatively porous, e.g., such that cells can penetrate into the device after insertion of the device. For example, in some cases, the polymeric constructs may be constructed and arranged within the device such that the device has an open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%. The “open porosity” is generally described as the volume of empty space within the device divided by the overall volume defined by the device, and can be thought of as being equivalent to void volume. Typically, the open porosity includes the volume within the device to which cells can access. In some cases, the device does not contain significant amounts of internal volume to which the cells are incapable of addressing, e.g., due to lack of access and/or pore access being too small.

In some cases, a “two-dimensional open porosity” may also be defined, e.g., of a device that is subsequently formed or manipulated into a 3-dimensional structure. The two-dimensional open porosities of a device can be defined as the void area within the two-dimensional configuration of the device (e.g., where no material is present) divided by the overall area of device, and can be determined before or after the device has been formed into a 3-dimensional structure. Depending on the application, a device may have a two-dimensional open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%, etc.

Another method of generally determining the two-dimensional porosity of the device is by determining the areal mass density, i.e., the mass of the device divided by the area of one face of the device (including holes or voids present therein). Thus, for example, in another set of embodiments, the device may have an areal mass density of less than about 100 micrograms/cm², less than about 80 micrograms/cm², less than about 60 micrograms/cm², less than about 50 micrograms/cm², less than about 40 micrograms/cm², less than about 30 micrograms/cm², or less than about 20 micrograms/cm².

The porosity of a device can be defined by one or more pores. Pores that are too small can hinder or restrict cell access. Thus, in one set of embodiments, the device may have an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm. However, in other embodiments, pores that are too big may prevent cells from being able to satisfactorily use or even access the pore volume. Thus, in some cases, the device may have an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers. Combinations of these are also possible, e.g., in one embodiment, the average pore size is at least about 100 micrometers and no more than about 1.5 mm. In addition, larger or smaller pores than these can also be used in a device in certain cases. Pore sizes may be determined using any suitable technique, e.g., through visual inspection (e.g., of microscope images), BET measurements, or the like.

In various embodiments, one or more of the polymers forming a polymeric construct may be a photoresist. While not commonly used in biological device s, photoresists are typically used in lithographic techniques, which can be used as discussed herein to form the polymeric construct. For example, the photoresist may be chosen for its ability to react to light to become substantially insoluble (or substantially soluble, in some cases) to a photoresist developer. For instance, photoresists that can be used within a polymeric construct include, but are not limited to, SU-8, S1805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, or the like. These and many other photoresists are available commercially.

A polymeric construct may also contain one or more polymers that are biocompatible and/or biodegradable, in certain embodiments. A polymer can be biocompatible, biodegradable, or both biocompatible and biodegradable, and in some cases, the degree of biodegradation or biocompatibility depends on the physiological environment to which the polymer is exposed to.

Typically, a biocompatible material is one that does not illicit an immune response, or elicits a relatively low immune response, e.g., one that does not impair the device or the cells therein from continuing to function for its intended use. In some embodiments, the biocompatible material is able to perform its desired function without eliciting any undesirable local or systemic effects in the subject. In some cases, the material can be incorporated into tissues within the subject, e.g., without eliciting any undesirable local or systemic effects, or such that any biological response by the subject does not substantially affect the ability of the material from continuing to function for its intended use. For example, in a device, the device may be able to determine cellular or tissue activity after insertion, e.g., without substantially eliciting undesirable effects in those cells, or undesirable local or systemic responses, or without eliciting a response that causes the device to cease functioning for its intended use. Examples of techniques for determining biocompatibility include, but are not limited to, the ISO 10993 series of for evaluating the biocompatibility of medical devices. As another example, a biocompatible material may be implanted in a subject for an extended period of time, e.g., at least about a month, at least about 6 months, or at least about a year, and the integrity of the material, or the immune response to the material, may be determined. For example, a suitably biocompatible material may be one in which the immune response is minimal, e.g., one that does not substantially harm the health of the subject. One example of a biocompatible material is poly(methyl methacrylate). In some embodiments, a biocompatible material may be used to cover or shield a non-biocompatible material (or a poorly biocompatible material) from the cells or tissue, for example, by covering the material.

A biodegradable material typically degrades over time when exposed to a biological system, e.g., through oxidation, hydrolysis, enzymatic attack, phagocytosis, or the like. For example, a biodegradable material can degrade over time when exposed to water (e.g., hydrolysis) or enzymes. In some cases, a biodegradable material is one that exhibits degradation (e.g., loss of mass and/or structure) when exposed to physiological conditions for at least about a month, at least about 6 months, or at least about a year. For example, the biodegradable material may exhibit a loss of mass of at least about 10%, at least about 20%, at least about 30%, at least about 40%, at least about 50%, at least about 60%, at least about 70%, at least about 80%, or at least about 90%. In certain cases, some or all of the degradation products may be resorbed or metabolized, e.g., into cells or tissues. For example, certain biodegradable materials, during degradation, release substances that can be metabolized by cells or tissues. For instance, polylactic acid releases water and lactic acid during degradation.

Examples of such biocompatible and/or biodegradable polymers include, but are not limited to, poly(lactic-co-glycolic acid), polylactic acid, polyglycolic acid, poly(methyl methacrylate), poly(trimethylene carbonate), collagen, fibrin, polysaccharidic materials such as chitosan or glycosaminoglycans, hyaluronic acid, polycaprolactone, and the like.

The polymers and other components forming the device can also be used in some embodiments to provide a certain degree of flexibility to the device, which can be quantified as a bending stiffness per unit width of polymer construct. In various embodiments, the overall device may have a bending stiffness of less than about 5 nN m, less than about 4.5 nN m, less than about 4 nN m, less than about 3.5 nN m, less than about 3 nN m, less than about 2.5 nN m, less than about 2 nN m, less than about 1.5 nN m, less than about 1 nN m, less than about 0.5 nM m, less than about 0.3 nM m, less than about 0.1 nM m, less than about 0.05 nM m, less than about 0.03 nM m, less than about 0.01 nM m, less than about 0.005 nM m, less than about 0.003 nM m, less than about 0.001 nM m, less than about 0.0005 nM m, less than about 0.0003 nM m, etc. In some cases, devices having relatively low bending stiffnesses are relatively flexible and bendable, and can be readily inserted into a tube, as discussed herein.

In some embodiments of the invention, the device may also contain other materials in addition to the photoresists or biocompatible and/or biodegradable polymers described above. Non-limiting examples include other polymers, growth hormones, extracellular matrix protein, specific metabolites or nutrients, or the like. For example, in one of embodiments, one or more agents able to promote cell growth can be added to the device, e.g., hormones such as growth hormones, extracellular matrix protein, pharmaceutical agents, vitamins, or the like. Many such growth hormones are commercially available, and may be readily selected by those of ordinary skill in the art based on the specific type of cell or tissue used or desired. Similarly, non-limiting examples of extracellular matrix proteins include gelatin, laminin, fibronectin, heparan sulfate, proteoglycans, entactin, hyaluronic acid, collagen, elastin, chondroitin sulfate, keratan sulfate, Matrigel™, or the like. Many such extracellular matrix proteins are available commercially, and also can be readily identified by those of ordinary skill in the art based on the specific type of cell or tissue used or desired.

As another example, in one set of embodiments, additional materials can be added to the device, e.g., to control the size of pores within the device, to promote cell adhesion or cell growth within the device, to increase the structural stability of the device, to control the flexibility of the device, etc. For instance, in one set of embodiments, additional fibers or other suitable polymers may be added to the device, e.g., electrospun fibers can be used as a secondary scaffold. The additional materials can be formed from any of the materials described herein, e.g., photoresists or biocompatible and/or biodegradable polymers, or other polymers described herein. As another non-limiting example, a glue such as a silicone elastomer glue can be used to control the shape of the device.

In some cases, the device can include a 2-dimensional structure that is formed into a final 3-dimensional structure, e.g., by folding or rolling the structure. It should be understood that although the 2-dimensional structure can be described as having an overall length, width, and height, the overall length and width of the structure may each be substantially greater than the overall height of the structure. The 2-dimensional structure may also be manipulated to have a different shape that is 3-dimensional, e.g., having an overall length, width, and height where the overall length and width of the structure are not each substantially greater than the overall height of the structure. For instance, the structure may be manipulated to increase the overall height of the material, relative to its overall length and/or width, for example, by folding or rolling the structure. Thus, for example, a relatively planar sheet of material (having a length and width much greater than its thickness) may be rolled up into a “tube,” such that the tube has an overall length, width, and height of relatively comparable dimensions).

Thus, for example, the 2-dimensional structure may comprise one or more nanoscale wires and one or more polymeric constructs formed into a 2-dimensional structure or network that is subsequently formed into a 3-dimensional structure. In some embodiments, the 2-dimensional structure may be rolled or curled up to form the 3-dimensional structure, or the 2-dimensional structure may be folded or creased one or more times to form the 3-dimensional structure. Such manipulations can be regular or irregular. In certain embodiments, as discussed herein, the manipulations are caused by pre-stressing the 2-dimensional structure such that it spontaneously forms the 3-dimensional structure, although in other embodiments, such manipulations can be performed separately, e.g., after formation of the 2-dimensional structure.

In some aspects, the device may include one or more metal leads or electrodes, or other conductive pathways. The metal leads or conductive pathways may provide mechanical support, and/or one or more metal leads can be used within a conductive pathway to a nanoscale wire. The metal lead may directly physically contact the nanoscale wire and/or there may be other materials between the metal lead and the nanoscale wire that allow electrical communication to occur. In some cases, one or more metal leads or other conductive pathways may extend such that the device can be connected to external electrical circuits, computers, or the like, e.g., as discussed herein. Metal leads are useful due to their high conductance, e.g., such that changes within electrical properties obtained from the conductive pathway can be related to changes in properties of the nanoscale wire, rather than changes in properties of the conductive pathway. However, it is not a requirement that only metal leads be used, and in other embodiments, other types of conductive pathways may also be used, in addition or instead of metal leads.

A wide variety of metal leads can be used, in various embodiments of the invention. As non-limiting examples, the metals used within a metal lead may include aluminum, gold, silver, copper, molybdenum, tantalum, titanium, nickel, tungsten, chromium, palladium, as well as any combinations of these and/or other metals. In some cases, the metal can be chosen to be one that is readily introduced into the device, e.g., using techniques compatible with lithographic techniques. For example, in one set of embodiments, lithographic techniques such as e-beam lithography, photolithography, X-ray lithography, extreme ultraviolet lithography, ion projection lithography, etc. may be used to layer or deposit one or more metals on a substrate. Additional processing steps can also be used to define or register the metal leads in some cases. Thus, for example, the thickness of a metal layer may be less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the layer may also be at least about 10 nm, at least about 20 nm, at least about 40 nm, at least about 60 nm, at least about 80 nm, or at least about 100 nm. For example, the thickness of a layer may be between about 40 nm and about 100 nm, between about 50 nm and about 80 nm.

In some embodiments, more than one metal can be used within a metal lead. For example, two, three, or more metals may be used within a metal lead. The metals may be deposited in different regions or alloyed together, or in some cases, the metals may be layered on top of each other, e.g., layered on top of each other using various lithographic techniques. For example, a second metal may be deposited on a first metal, and in some cases, a third metal may be deposited on the second metal, etc. Additional layers of metal (e.g., fourth, fifth, sixth, etc.) may also be used in some embodiments. The metals can all be different, or in some cases, some of the metals (e.g., the first and third metals) may be the same. Each layer may independently be of any suitable thickness or dimension, e.g., of the dimensions described above, and the thicknesses of the various layers can independently be the same or different.

If dissimilar metals are layered on top of each other, they may be layered in some embodiments in a “stressed” configuration (although in other embodiments they may not necessarily be stressed). As a specific non-limiting example, chromium and palladium can be layered together to cause stresses in the metal leads to occur, thereby causing warping or bending of the metal leads. The amount and type of stress may also be controlled, e.g., by controlling the thicknesses of the layers. For example, relatively thinner layers can be used to increase the amount of warping that occurs.

Without wishing to be bound by any theory, it is believed that layering metals having a difference in stress (e.g., film stress) with respect to each other may, in some cases, cause stresses within the metal, which can cause bending or warping as the metals seek to relieve the stresses. In some embodiments, such mismatches are undesirable because they could cause warping of the metal leads and thus, the device. However, in other embodiments, such mismatches may be desired, e.g., so that the device can be intentionally deformed to form a 3-dimensional structure, as discussed below. In addition, in certain embodiments, the deposition of mismatched metals within a lead may occur at specific locations within the device, e.g., to cause specific warpings to occur, which can be used to cause the device to be deformed into a particular shape or configuration. For example, a “line” of such mismatches can be used to cause an intentional bending or folding along the line of the device.

The device may include one or more nanoscale wires, which may be the same or different from each other, in accordance with various aspects of the invention. Non-limiting examples of such nanoscale wires are discussed in detail below, and include, for instance, semiconductor nanowires, carbon nanotubes, or the like. In some cases, at least one of the nanoscale wires is a silicon nanowire. The nanoscale wires may also be straight, or kinked in some cases. In some embodiments, one or more of the nanoscale wires may form at least a portion of a transistor, such as a field-effect transistor, e.g., as is discussed in more detail below. The nanoscale wires may be distributed within the device in any suitable configuration, for example, in an ordered array or randomly distributed. In some cases, the nanoscale wires are distributed such that an increasing concentration of nanoscale wires can be found towards the portion of the device that is first inserted.

In some cases, some or all of the nanoscale wires are individually electronically addressable within the device. For instance, in some cases, at least about 10%, at least about 20%, at least about 30%, at least about 40%, at least about 50%, at least about 60%, at least about 70%, at least about 80%, at least about 90%, or substantially all of the nanoscale wires may be individually electronically addressable. In some embodiments, an electrical property of a nanoscale wire can be individually determinable (e.g., being partially or fully resolvable without also including the electrical properties of other nanoscale wires), and/or such that the electrical property of a nanoscale wire may be individually controlled (for example, by applying a desired voltage or current to the nanoscale wire, for instance, without simultaneously applying the voltage or current to other nanoscale wires). In other embodiments, however, at least some of the nanoscale wires can be controlled within the same electronic circuit (e.g., by incorporating the nanoscale wires in series and/or in parallel), such that the nanoscale wires can still be electronically controlled and/or determined.

In various embodiments, more than one nanoscale wire may be present within the device. The nanoscale wires may each independently be the same or different. For example, the device may comprise at least 5 nanoscale wires, at least about 10 nanoscale wires, at least about 15 nanoscale wires, at least about 20 nanoscale wires, at least about 25 nanoscale wires, at least about 30 nanoscale wires, at least about 50 nanoscale wires, at least about 100 nanoscale wires, at least about 300 nanoscale wires, at least about 1000 nanoscale wires, etc.

In addition, in some embodiments, there may be a relatively high density of nanoscale wires within the device, or at least a portion of the device. The nanoscale wires may be distributed uniformly or non-uniformly on the device. In some cases, the nanoscale wires may be distributed at an average density of at least about 5 wires/mm², at least about 10 wires/mm², at least about 30 wires/mm², at least about 50 wires/mm², at least about 75 wires/mm², at least about 100 wires/mm², at least about 300 wires/mm², at least about 500 wires/mm², at least about 750 wires/mm², at least about 1000 wires/mm², etc. In certain embodiments, the nanoscale wires are distributed such that the average separation between a nanoscale wire and its nearest neighboring nanoscale wire is less than about 2 mm, less than about 1 mm, less than about 500 micrometers, less than about 300 micrometers, less than about 100 micrometers, less than about 50 micrometers, less than about 30 micrometers, or less than about 10 micrometers.

Some or all of the nanoscale wires may be in electrical communication with one or more electrical connectors via one or more conductive pathways. The electrical connectors may be positioned on a portion of the device that is not inserted into the tissue. The electrical connectors may be made out of any suitable material that allows transmission of an electrical signal. For example, the electrical connectors may comprise gold, silver, copper, aluminum, tantalum, titanium, nickel, tungsten, chromium, palladium, etc. In some cases, the electrical connectors have an average cross-section of less than about 10 micrometers, less than about 8 micrometers, less than about 6 micrometers, less than about 5 micrometers, less than about 4 micrometers, less than about 3 micrometers, less than about 2 micrometers, less than about 1 micrometer, etc.

In some embodiments, the electrical connectors can be used to determine a property of a nanoscale wire within the device (for example, an electrical property or a chemical property as is discussed herein), and/or to direct an electrical signal to a nanoscale wire, e.g., to electrically stimulate cells proximate the nanoscale wire. The conductive pathways can form an electrical circuit that is internally contained within the device, and/or that extends externally of the device, e.g., such that the electrical circuit is in electrical communication with an external electrical system, such as a computer or a transmitter (for instance, a radio transmitter, a wireless transmitter, an Internet connection, etc.). Any suitable pathway conductive pathway may be used, for example, pathways comprising metals, semiconductors, conductive polymers, or the like.

Furthermore, more than one conductive pathway may be used in certain embodiments. For example, multiple conductive pathways can be used such that some or all of the nanoscale wires within the device may be electronically individually addressable, as previously discussed. However, in other embodiments, more than one nanoscale wire may be addressable by a particular conductive pathway. In addition, in some cases, other electronic components may also be present within the device, e.g., as part of a conductive pathway or otherwise forming part of an electrical circuit. Examples include, but are not limited to, transistors such as field-effect transistors or bipolar junction transistors, resistors, capacitors, inductors, diodes, integrated circuits, etc. In certain cases, some of these may also comprise nanoscale wires. For example, in some embodiments, two sets of electrical connectors and conductive pathways, and a nanoscale wire, may be used to define a transistor such as a field effect transistor, e.g., where the nanoscale wire defines the gate. As mentioned, the environment in and/or around the nanoscale wire can affect the ability of the nanoscale wire to function as a gate.

As mentioned, in various embodiments, one or more electrodes, electrical connectors, and/or conductive pathways may be positioned in electrical and/or physical communication with the nanoscale wires. These can be patterned to be in direct physical contact the nanoscale wire and/or there may be other materials that allow electrical communication to occur. Metals may be used due to their high conductance, e.g., such that changes within electrical properties obtained from the conductive pathway may be related to changes in properties of the nanoscale wire, rather than changes in properties of the conductive pathway. However, in other embodiments, other types of electrode materials are used, in addition or instead of metals.

A wide variety of metals may be used in various embodiments of the invention, for example in an electrode, electrical connector, conductive pathway, metal construct, polymer construct, etc. As non-limiting examples, the metals may include one or more of aluminum, gold, silver, copper, molybdenum, tantalum, titanium, nickel, tungsten, chromium, palladium, as well as any combinations of these and/or other metals. In some cases, the metal may be chosen to be one that is readily introduced, e.g., using techniques compatible with lithographic techniques. For example, in one set of embodiments, lithographic techniques such as e-beam lithography, photolithography, X-ray lithography, extreme ultraviolet lithography, ion projection lithography, etc. can be used to pattern or deposit one or more metals.

Additional processing steps can also be used to define or register the electrode, electrical connector, conductive pathway, metal construct, polymer construct, etc. in some cases. Thus, for example, the thickness of one of these may be less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the electrode may also be at least about 10 nm, at least about 20 nm, at least about 40 nm, at least about 60 nm, at least about 80 nm, or at least about 100 nm. For example, the thickness of an electrode may be between about 40 nm and about 100 nm, between about 50 nm and about 80 nm.

In some embodiments, more than one metal may be used. The metals can be deposited in different regions or alloyed together, or in some cases, the metals may be layered on top of each other, e.g., layered on top of each other using various lithographic techniques. For example, a second metal may be deposited on a first metal, and in some cases, a third metal may be deposited on the second metal, etc. Additional layers of metal (e.g., fourth, fifth, sixth, etc.) can also be used in some embodiments. The metals may all be different, or in some cases, some of the metals (e.g., the first and third metals) may be the same. Each layer may independently be of any suitable thickness or dimension, e.g., of the dimensions described above, and the thicknesses of the various layers may independently be the same or different.

As mentioned, any nanoscale wire can be used in the device. Non-limiting examples of suitable nanoscale wires include carbon nanotubes, nanorods, nanowires, organic and inorganic conductive and semiconducting polymers, metal nanoscale wires, semiconductor nanoscale wires (for example, formed from silicon), and the like. If carbon nanotubes are used, they may be single-walled and/or multi-walled, and may be metallic and/or semiconducting in nature. Other conductive or semiconducting elements that may not be nanoscale wires, but are of various small nanoscopic-scale dimension, also can be used in certain embodiments.

In general, a “nanoscale wire” (also known herein as a “nanoscopic-scale wire” or “nanoscopic wire”) generally is a wire or other nanoscale object, that at any point along its length, has at least one cross-sectional dimension and, in some embodiments, two orthogonal cross-sectional dimensions (e.g., a diameter) of less than 1 micrometer, less than about 500 nm, less than about 200 nm, less than about 150 nm, less than about 100 nm, less than about 70, less than about 50 nm, less than about 20 nm, less than about 10 nm, less than about 5 nm, than about 2 nm, or less than about 1 nm. In some embodiments, the nanoscale wire is generally cylindrical. In other embodiments, however, other shapes are possible; for example, the nanoscale wire can be faceted, i.e., the nanoscale wire may have a polygonal cross-section. The cross-section of a nanoscale wire can be of any arbitrary shape, including, but not limited to, circular, square, rectangular, annular, polygonal, or elliptical, and may be a regular or an irregular shape. The nanoscale wire can also be solid or hollow.

In some cases, the nanoscale wire has one dimension that is substantially longer than the other dimensions of the nanoscale wire. For example, the nanoscale wire may have a longest dimension that is at least about 1 micrometer, at least about 3 micrometers, at least about 5 micrometers, or at least about 10 micrometers or about 20 micrometers in length, and/or the nanoscale wire may have an aspect ratio (longest dimension to shortest orthogonal dimension) of greater than about 2:1, greater than about 3:1, greater than about 4:1, greater than about 5:1, greater than about 10:1, greater than about 25:1, greater than about 50:1, greater than about 75:1, greater than about 100:1, greater than about 150:1, greater than about 250:1, greater than about 500:1, greater than about 750:1, or greater than about 1000:1 or more in some cases.

In some embodiments, a nanoscale wire are substantially uniform, or have a variation in average diameter of the nanoscale wire of less than about 30%, less than about 25%, less than about 20%, less than about 15%, less than about 10%, or less than about 5%. For example, the nanoscale wires may be grown from substantially uniform nanoclusters or particles, e.g., colloid particles. See, e.g., U.S. Pat. No. 7,301,199, issued Nov. 27, 2007, entitled “Nanoscale Wires and Related Devices,” by Lieber, et al., incorporated herein by reference in its entirety. In some cases, the nanoscale wire may be one of a population of nanoscale wires having an average variation in diameter, of the population of nanowires, of less than about 30%, less than about 25%, less than about 20%, less than about 15%, less than about 10%, or less than about 5%.

In some embodiments, a nanoscale wire has a conductivity of or of similar magnitude to any semiconductor or any metal. The nanoscale wire can be formed of suitable materials, e.g., semiconductors, metals, etc., as well as any suitable combinations thereof. In some cases, the nanoscale wire will have the ability to pass electrical charge, for example, being electrically conductive. For example, the nanoscale wire may have a relatively low resistivity, e.g., less than about 10⁻³ Ohm m, less than about 10⁻⁴ Ohm m, less than about 10⁻⁶ Ohm m, or less than about 10⁻⁷ Ohm m. The nanoscale wire can, in some embodiments, have a conductance of at least about 1 microsiemens, at least about 3 microsiemens, at least about 10 microsiemens, at least about 30 microsiemens, or at least about 100 microsiemens.

The nanoscale wire can be solid or hollow, in various embodiments. As used herein, a “nanotube” is a nanoscale wire that is hollow, or that has a hollowed-out core, including those nanotubes known to those of ordinary skill in the art. As another example, a nanotube may be created by creating a core/shell nanowire, then etching away at least a portion of the core to leave behind a hollow shell. Accordingly, in one set of embodiments, the nanoscale wire is a non-carbon nanotube. In contrast, a “nanowire” is a nanoscale wire that is typically solid (i.e., not hollow). Thus, in one set of embodiments, the nanoscale wire may be a semiconductor nanowire, such as a silicon nanowire.

In one set of embodiment, a nanoscale wire may comprise or consist essentially of a metal. Non-limiting examples of potentially suitable metals include aluminum, gold, silver, copper, molybdenum, tantalum, titanium, nickel, tungsten, chromium, or palladium. In another set of embodiments, a nanoscale wire comprises or consists essentially of a semiconductor. Typically, a semiconductor is an element having semiconductive or semi-metallic properties (i.e., between metallic and non-metallic properties). An example of a semiconductor is silicon. Other non-limiting examples include elemental semiconductors, such as gallium, germanium, diamond (carbon), tin, selenium, tellurium, boron, or phosphorous. In other embodiments, more than one element may be present in the nanoscale wire as the semiconductor, for example, gallium arsenide, gallium nitride, indium phosphide, cadmium selenide, etc. Still other examples include a Group II-VI material (which includes at least one member from Group II of the Periodic Table and at least one member from Group VI, for example, ZnS, ZnSe, ZnSSe, ZnCdS, CdS, or CdSe), or a Group III-V material (which includes at least one member from Group III and at least one member from Group V, for example GaAs, GaP, GaAsP, InAs, InP, AlGaAs, or InAsP). In some cases, at least one of the nanoscale wires is a silicon nanowire.

In certain embodiments, the semiconductor can be undoped or doped (e.g., p-type or n-type). For example, in one set of embodiments, a nanoscale wire may be a p-type semiconductor nanoscale wire or an n-type semiconductor nanoscale wire, and can be used as a component of a transistor such as a field effect transistor (“FET”). For instance, the nanoscale wire may act as the “gate” of a source-gate-drain arrangement of a FET, while metal leads or other conductive pathways (as discussed herein) are used as the source and drain electrodes.

In some embodiments, a dopant or a semiconductor may include mixtures of Group IV elements, for example, a mixture of silicon and carbon, or a mixture of silicon and germanium. In other embodiments, the dopant or the semiconductor may include a mixture of a Group III and a Group V element, for example, BN, BP, BAs, AN, AlP, AlAs, AlSb, GaN, GaP, GaAs, GaSb, InN, InP, InAs, or InSb. Mixtures of these may also be used, for example, a mixture of BN/BP/BAs, or BN/A1P. In other embodiments, the dopants may include alloys of Group III and Group V elements. For example, the alloys may include a mixture of AlGaN, GaPAs, InPAs, GaInN, AlGaInN, GaInAsP, or the like. In other embodiments, the dopants may also include a mixture of Group II and Group VI semiconductors. For example, the semiconductor may include ZnO, ZnS, ZnSe, ZnTe, CdS, CdSe, CdTe, HgS, HgSe, HgTe, BeS, BeSe, BeTe, MgS, MgSe, or the like. Alloys or mixtures of these dopants are also be possible, for example, (ZnCd)Se, or Zn(SSe), or the like. Additionally, alloys of different groups of semiconductors may also be possible, for example, a combination of a Group II-Group VI and a Group III-Group V semiconductor, for example, (GaAs)ₓ(ZnS)₁₋ₓ. Other examples of dopants may include combinations of Group IV and Group VI elemnts, such as GeS, GeSe, GeTe, SnS, SnSe, SnTe, PbO, PbS, PbSe, or PbTe. Other semiconductor mixtures may include a combination of a Group I and a Group VII, such as CuF, CuCl, CuBr, Cut AgF, AgCl, AgBr, AgI, or the like. Other dopant compounds may include different mixtures of these elements, such as BeSiN₂, CaCN₂, ZnGeP₂, CdSnAs₂, ZnSnSb₂, CuGeP₃, CuSi₂P₃, Si₃N₄, Ge₃N₄, Al₂O₃, (Al, Ga, In)₂(S, Se, Te)₃, Al₂CO, (Cu, Ag)(Al, Ga, In, Tl, Fe)(S, Se, Te)₂ and the like.

The doping of the semiconductor to produce a p-type or n-type semiconductor may be achieved via bulk-doping in certain embodiments, although in other embodiments, other doping techniques (such as ion implantation) can be used. Many such doping techniques that can be used will be familiar to those of ordinary skill in the art, including both bulk doping and surface doping techniques. A bulk-doped article (e.g. an article, or a section or region of an article) is an article for which a dopant is incorporated substantially throughout the crystalline lattice of the article, as opposed to an article in which a dopant is only incorporated in particular regions of the crystal lattice at the atomic scale, for example, only on the surface or exterior. For example, some articles are typically doped after the base material is grown, and thus the dopant only extends a finite distance from the surface or exterior into the interior of the crystalline lattice. It should be understood that “bulk-doped” does not define or reflect a concentration or amount of doping in a semiconductor, nor does it necessarily indicate that the doping is uniform. “Heavily doped” and “lightly doped” are terms the meanings of which are clearly understood by those of ordinary skill in the art. In some embodiments, one or more regions comprise a single monolayer of atoms (“delta-doping”). In certain cases, the region may be less than a single monolayer thick (for example, if some of the atoms within the monolayer are absent). As a specific example, the regions may be arranged in a layered structure within the nanoscale wire, and one or more of the regions can be delta-doped or partially delta-doped.

Accordingly, in one set of embodiments, the nanoscale wires may include a heterojunction, e.g., of two regions with dissimilar materials or elements, and/or the same materials or elements but at different ratios or concentrations. The regions of the nanoscale wire may be distinct from each other with minimal cross-contamination, or the composition of the nanoscale wire can vary gradually from one region to the next. The regions may be both longitudinally arranged relative to each other, or radially arranged (e.g., as in a core/shell arrangement) on the nanoscale wire. Each region may be of any size or shape within the wire. The junctions may be, for example, a p/n junction, a p/p junction, an n/n junction, a p/i junction (where i refers to an intrinsic semiconductor), an n/i junction, an i/i junction, or the like. The junction can also be a Schottky junction in some embodiments. The junction may also be, for example, a semiconductor/semiconductor junction, a semiconductor/metal junction, a semiconductor/insulator junction, a metal/metal junction, a metal/insulator junction, an insulator/insulator junction, or the like. The junction may also be a junction of two materials, a doped semiconductor to a doped or an undoped semiconductor, or a junction between regions having different dopant concentrations. The junction can also be a defected region to a perfect single crystal, an amorphous region to a crystal, a crystal to another crystal, an amorphous region to another amorphous region, a defected region to another defected region, an amorphous region to a defected region, or the like. More than two regions may be present, and these regions may have unique compositions or may comprise the same compositions. As one example, a wire can have a first region having a first composition, a second region having a second composition, and a third region having a third composition or the same composition as the first composition. Non-limiting examples of nanoscale wires comprising heterojunctions (including core/shell heterojunctions, longitudinal heterojunctions, etc., as well as combinations thereof) are discussed in U.S. Pat. No. 7,301,199, issued Nov. 27, 2007, entitled “Nanoscale Wires and Related Devices,” by Lieber, et al., incorporated herein by reference in its entirety.

In some embodiments, the nanoscale wire is a bent or a kinked nanoscale wire. A kink is typically a relatively sharp transition or turning between a first substantially straight portion of a wire and a second substantially straight portion of a wire. For example, a nanoscale wire may have 1, 2, 3, 4, or 5 or more kinks. In some cases, the nanoscale wire is formed from a single crystal and/or comprises or consists essentially of a single crystallographic orientation, for example, a <110> crystallographic orientation, a <112> crystallographic orientation, or a <1120> crystallographic orientation. It should be noted that the kinked region need not have the same crystallographic orientation as the rest of the semiconductor nanoscale wire. In some embodiments, a kink in the semiconductor nanoscale wire may be at an angle of about 120° or a multiple thereof. The kinks can be intentionally positioned along the nanoscale wire in some cases. For example, a nanoscale wire may be grown from a catalyst particle by exposing the catalyst particle to various gaseous reactants to cause the formation of one or more kinks within the nanoscale wire. Non-limiting examples of kinked nanoscale wires, and suitable techniques for making such wires, are disclosed in International Patent Application No. PCT/US2010/050199, filed Sep. 24, 2010, entitled “Bent Nanowires and Related Probing of Species,” by Tian, et al., published as WO 2011/038228 on Mar. 31, 2011, incorporated herein by reference in its entirety.

In one set of embodiments, the nanoscale wire is formed from a single crystal, for example, a single crystal nanoscale wire comprising a semiconductor. A single crystal item may be formed via covalent bonding, ionic bonding, or the like, and/or combinations thereof. While such a single crystal item may include defects in the crystal in some cases, the single crystal item is distinguished from an item that includes one or more crystals, not ionically or covalently bonded, but merely in close proximity to one another.

In some embodiments, the nanoscale wires used herein are individual or free-standing nanoscale wires. For example, an “individual” or a “free-standing” nanoscale wire may, at some point in its life, not be attached to another article, for example, with another nanoscale wire, or the free-standing nanoscale wire may be in solution. This is in contrast to nanoscale features etched onto the surface of a substrate, e.g., a silicon wafer, in which the nanoscale features are never removed from the surface of the substrate as a free-standing article. This is also in contrast to conductive portions of articles which differ from surrounding material only by having been altered chemically or physically, in situ, i.e., where a portion of a uniform article is made different from its surroundings by selective doping, etching, etc. An “individual” or a “free-standing” nanoscale wire is one that can be (but need not be) removed from the location where it is made, as an individual article, and transported to a different location and combined with different components to make a functional device such as those described herein and those that would be contemplated by those of ordinary skill in the art upon reading this disclosure.

The nanoscale wire, in some embodiments, may be responsive to a property external of the nanoscale wire, e.g., a chemical property, an electrical property, a physical property, etc. Such determination may be qualitative and/or quantitative, and such determinations may also be recorded, e.g., for later use. For example, in one set of embodiments, the nanoscale wire may be responsive to voltage. For instance, the nanoscale wire may exhibits a voltage sensitivity of at least about 5 microsiemens/V; by determining the conductivity of a nanoscale wire, the voltage surrounding the nanoscale wire may thus be determined. In other embodiments, the voltage sensitivity can be at least about 10 microsiemens/V, at least about 30 microsiemens/V, at least about 50 microsiemens/V, or at least about 100 microsiemens/V. Other examples of electrical properties that can be determined include resistance, resistivity, conductance, conductivity, impendence, or the like.

As another example, a nanoscale wire may be responsive to a chemical property of the environment surrounding the nanoscale wire. For example, an electrical property of the nanoscale wire can be affected by a chemical environment surrounding the nanoscale wire, and the electrical property can be thereby determined to determine the chemical environment surrounding the nanoscale wire. As a specific non-limiting example, the nanoscale wires may be sensitive to pH or hydrogen ions. Further non-limiting examples of such nanoscale wires are discussed in U.S. Pat. No. 7,129,554, filed Oct. 31, 2006, entitled “Nanosensors,” by Lieber, et al., incorporated herein by reference in its entirety.

As a non-limiting example, the nanoscale wire may have the ability to bind to an analyte indicative of a chemical property of the environment surrounding the nanoscale wire (e.g., hydrogen ions for pH, or concentration for an analyte of interest), and/or the nanoscale wire may be partially or fully functionalized, i.e. comprising surface functional moieties, to which an analyte is able to bind, thereby causing a determinable property change to the nanoscale wire, e.g., a change to the resistivity or impedance of the nanoscale wire. The binding of the analyte can be specific or non-specific. Functional moieties may include simple groups, selected from the groups including, but not limited to, —OH, —CHO, —COOH, SO₃H, —CN, —NH₂, —SH, —COSH, —COOR, halide; biomolecular entities including, but not limited to, amino acids, proteins, sugars, DNA, antibodies, antigens, and enzymes; grafted polymer chains with chain length less than the diameter of the nanowire core, selected from a group of polymers including, but not limited to, polyamide, polyester, polyimide, polyacrylic; a shell of material comprising, for example, metals, semiconductors, and insulators, which may be a metallic element, an oxide, an sulfide, a nitride, a selenide, a polymer and a polymer gel. A non-limiting example of a protein is PSA (prostate specific antigen), which can be determined, for example, by modifying the nanoscale wires by binding monoclonal antibodies for PSA (Ab1) thereto. See, e.g., U.S. Pat. No. 8,232,584, issued Jul. 31, 2012, entitled “Nanoscale Sensors,” by Lieber, et al., incorporated herein by reference in its entirety.

In some embodiments, a reaction entity may be bound to a surface of the nanoscale wire, and/or positioned in relation to the nanoscale wire such that the analyte can be determined by determining a change in a property of the nanoscale wire. The “determination” may be quantitative and/or qualitative, depending on the application, and in some cases, the determination may also be analyzed, recorded for later use, transmitted, or the like. The term “reaction entity” refers to any entity that can interact with an analyte in such a manner to cause a detectable change in a property (such as an electrical property) of a nanoscale wire. The reaction entity may enhance the interaction between the nanowire and the analyte, or generate a new chemical species that has a higher affinity to the nanowire, or to enrich the analyte around the nanowire. The reaction entity can comprise a binding partner to which the analyte binds. The reaction entity, when a binding partner, can comprise a specific binding partner of the analyte. For example, the reaction entity may be a nucleic acid, an antibody, a sugar, a carbohydrate or a protein. Alternatively, the reaction entity may be a polymer, catalyst, or a quantum dot. A reaction entity that is a catalyst can catalyze a reaction involving the analyte, resulting in a product that causes a detectable change in the nanowire, e.g. via binding to an auxiliary binding partner of the product electrically coupled to the nanowire. Another exemplary reaction entity is a reactant that reacts with the analyte, producing a product that can cause a detectable change in the nanowire. The reaction entity can comprise a shell on the nanowire, e.g. a shell of a polymer that recognizes molecules in, e.g., a gaseous sample, causing a change in conductivity of the polymer which, in turn, causes a detectable change in the nanowire.

The term “binding partner” refers to a molecule that can undergo binding with a particular analyte, or “binding partner” thereof, and includes specific, semi-specific, and non-specific binding partners as known to those of ordinary skill in the art. The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen. Other examples include, nucleic acids that specifically bind (hybridize) to their complement, antibodies specifically bind to their antigen, and the like. The binding may be by one or more of a variety of mechanisms including, but not limited to ionic interactions, and/or covalent interactions, and/or hydrophobic interactions, and/or van der Waals interactions, etc.

The antibody may be any protein or glycoprotein comprising or consisting essentially of one or more polypeptides substantially encoded by immunoglobulin genes or fragments of immunoglobulin genes. Examples of recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′₂, a dimer of Fab which itself is a light chain joined to VHCH1 by a disulfide bond. The F(ab)′₂ may be reduced under mild conditions to break the disulfide linkage in the hinge region thereby converting the (Fab)₂ dimer into an Fab′ monomer. The Fab′ monomer is essentially a Fab with part of the hinge region. While various antibody fragments are defined in terms of the digestion of an intact antibody, one of skill will appreciate that such fragments may be synthesized de novo either chemically, by utilizing recombinant DNA methodology, or by “phage display” methods. Non-limiting examples of antibodies include single chain antibodies, e.g., single chain Fv (scFv) antibodies in which a variable heavy and a variable light chain are joined together (directly or through a peptide linker) to form a continuous polypeptide.

Thus, in some embodiments, a property such as a chemical property and/or an electrical property can be determined, e.g., at a resolution of less than about 2 mm, less than about 1 mm, less than about 500 micrometers, less than about 300 micrometers, less than about 100 micrometers, less than about 50 micrometers, less than about 30 micrometers, or less than about 10 micrometers, etc., e.g., due to the average separation between a nanoscale wire and its nearest neighboring nanoscale wire. In addition, the property may be determined within the tissue in 3 dimensions in some instances, in contrast with many other techniques where only a surface of the biological tissue can be studied. Accordingly, very high resolution and/or 3-dimensional mappings of the property of the biological tissue can be obtained in some embodiments. Any suitable tissue may be studied, e.g., brain tissue, cardiac tissue, vascular tissue, muscle, cartilage, bone, liver tissue, pancreatic tissue, bladder tissue, airway tissues, bone marrow tissue, or the like.

In addition, in some cases, such properties can be determined and/or recorded as a function of time. Thus, for example, such properties can be determined at a time resolution of less than about 1 min, less than about 30 s, less than about 15 s, less than about 10 s, less than about 5 s, less than about 3 s, less than about 1 s, less than about 500 ms, less than about 300 ms, less than about 100 ms, less than about 50 ms, less than about 30 ms, less than about 10 ms, less than about 5 ms, less than about 3 ms, less than about 1 ms, etc.

In yet another set of embodiments, the biological tissue, and/or portions of the biological tissue, may be electrically stimulated using nanoscale wires present within the tissue. For example, all, or a subset of the electrically active nanoscale wires may be electrically stimulated, e.g., by using an external electrical system, such as a computer. Thus, for example, a single nanoscale wire, a group of nanoscale wires, or substantially all of the nanoscale wires can be electrically stimulated, depending on the particular application. In some cases, such nanoscale wires can be stimulated in a particular pattern, e.g., to cause cardiac or muscle cells to contract or beat in a particular pattern (for example, as part of a prosthetic or a pacemaker), to cause the firing of neurons with a particular pattern, to monitor the status of an implanted tissue within a subject, or the like.

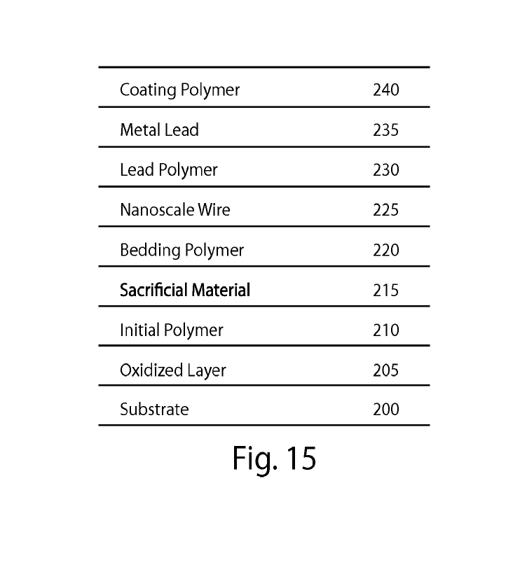

Another aspect of the present invention is generally directed to systems and methods for making and using such devices, e.g., for insertion into matter. Briefly, in one set of embodiments, a device can be constructed by assembling various polymers, metals, nanoscale wires, and other components together on a substrate. For example, lithographic techniques such as e-beam lithography, photolithography, X-ray lithography, extreme ultraviolet lithography, ion projection lithography, etc. may be used to pattern polymers, metals, etc. on the substrate, and nanoscale wires can be prepared separately then added to the substrate. After assembly, at least a portion of the substrate (e.g., a sacrificial material) may be removed, allowing the device to be partially or completely removed from the substrate. The device can, in some cases, be formed into a 3-dimensional structure, for example, spontaneously, or by folding or rolling the structure. Other materials may also be added to the device, e.g., to help stabilize the structure, to add additional agents to enhance its biocompatibility, etc. The device can be used in vivo, e.g., by implanting it in a subject, and/or in vitro, e.g., by seeding cells, etc. on the device. In addition, in some cases, cells may initially be grown on the device before the device is implanted into a subject. A schematic diagram of the layers formed on the substrate in one embodiment is shown in FIG. 15. However, it should be understood that this diagram is illustrative only and is not drawn to scale, and not all of the layers shown in FIG. 15 are necessarily required in every embodiment of the invention.

The substrate (200 in FIG. 15) may be chosen to be one that can be used for lithographic techniques such as e-beam lithography or photolithography, or other lithographic techniques including those discussed herein. For example, the substrate may comprise or consist essentially of a semiconductor material such as silicon, although other substrate materials (e.g., a metal) can also be used. Typically, the substrate is one that is substantially planar, e.g., so that polymers, metals, and the like can be patterned on the substrate.

In some cases, a portion of the substrate can be oxidized, e.g., forming SiO₂ and/or Si₃N₄ on a portion of the substrate, which may facilitate subsequent addition of materials (metals, polymers, etc.) to the substrate. In some cases, the oxidized portion may form a layer of material on the substrate (205 in FIG. 15), e.g., having a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

In certain embodiments, one or more polymers can also be deposited or otherwise formed prior to depositing the sacrificial material. In some cases, the polymers may be deposited or otherwise formed as a layer of material (210 in FIG. 15) on the substrate. Deposition may be performed using any suitable technique, e.g., using lithographic techniques such as e-beam lithography, photolithography, X-ray lithography, extreme ultraviolet lithography, ion projection lithography, etc. In some cases, some or all of the polymers may be biocompatible and/or biodegradable. The polymers that are deposited may also comprise methyl methacrylate and/or poly(methyl methacrylate), in some embodiments. One, two, or more layers of polymer can be deposited (e.g., sequentially) in various embodiments, and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Next, a sacrificial material may be deposited. The sacrificial material can be chosen to be one that can be removed without substantially altering other materials (e.g., polymers, other metals, nanoscale wires, etc.) deposited thereon. For example, in one embodiment, the sacrificial material may be a metal, e.g., one that is easily etchable. For instance, the sacrificial material can comprise germanium or nickel, which can be etched or otherwise removed, for example, using a peroxide (e.g., H₂O₂) or a nickel etchant (many of which are readily available commercially). In some cases, the sacrificial material may be deposited on oxidized portions or polymers previously deposited on the substrate. In some cases, the sacrificial material is deposited as a layer (e.g., 215 in FIG. 15). The layer can have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

In some embodiments, a “bedding” polymer can be deposited, e.g., on the sacrificial material. The bedding polymer may include one or more polymers, which may be deposited as one or more layers (220 in FIG. 15). The bedding polymer can be used to support the nanoscale wires, and in some cases, partially or completely surround the nanoscale wires, depending on the application. For example, as discussed below, one or more nanoscale wires may be deposited on at least a portion of the uppermost layer of bedding polymer.

For instance, the bedding polymer can at least partially define a device. In one set of embodiments, the bedding polymer may be deposited as a layer of material, such that portions of the bedding polymer may be subsequently removed. For example, the bedding polymer can be deposited using lithographic techniques such as e-beam lithography, photolithography, X-ray lithography, extreme ultraviolet lithography, ion projection lithography, etc., or using other techniques for removing polymer that are known to those of ordinary skill in the art. In some cases, more than one bedding polymer is used, e.g., deposited as more than one layer (e.g., sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc. For example, in some embodiments, portions of the photoresist may be exposed to light (visible, UV, etc.), electrons, ions, X-rays, etc. (e.g., projected onto the photoresist), and the exposed portions can be etched away (e.g., using suitable etchants, plasma, etc.) to produce the pattern.

Accordingly, the bedding polymer may be formed into a particular pattern, e.g., in a grid, or in a pattern that suggests an endogenous probe, before or after deposition of nanoscale wires (as discussed in detail below), in certain embodiments of the invention. The pattern can be regular or irregular. For example, the bedding polymer can be formed into a pattern defining pore sizes such as those discussed herein. For instance, the polymer may have an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm, and/or an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers, etc.

Any suitable polymer may be used as the bedding polymer. In some cases, one or more of the polymers can be chosen to be biocompatible and/or biodegradable. In certain embodiments, one or more of the bedding polymers may comprise a photoresist. Photoresists can be useful due to their familiarity in use in lithographic techniques such as those discussed herein. Non-limiting examples of photoresists include SU-8, S1805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, etc., as well as any others discussed herein.

In certain embodiments, one or more of the bedding polymers can be heated or baked, e.g., before or after depositing nanoscale wires thereon as discussed below, and/or before or after patterning the bedding polymer. For example, such heating or baking, in some cases, is important to prepare the polymer for lithographic patterning. In various embodiments, the bedding polymer may be heated to a temperature of at least about 30° C., at least about 65° C., at least about 95° C., at least about 150° C., or at least about 180° C., etc.

Next, one or more nanoscale wires (e.g., 225 in FIG. 15) may be deposited, e.g., on a bedding polymer on the substrate. Any of the nanoscale wires described herein may be used, e.g., n-type and/or p-type nanoscale wires, substantially uniform nanoscale wires (e.g., having a variation in average diameter of less than 20%), nanoscale wires having a diameter of less than about 1 micrometer, semiconductor nanowires, silicon nanowires, bent nanoscale wires, kinked nanoscale wires, core/shell nanowires, nanoscale wires with heterojunctions, etc. In some cases, the nanoscale wires are present in a liquid which is applied to the substrate, e.g., poured, painted, or otherwise deposited thereon. In some embodiments, the liquid is chosen to be relatively volatile, such that some or all of the liquid can be removed by allowing it to substantially evaporate, thereby depositing the nanoscale wires. In some cases, at least a portion of the liquid can be dried off, e.g., by applying heat to the liquid. Examples of suitable liquids include water or isopropanol.

In some cases, at least some of the nanoscale wires may be at least partially aligned, e.g., as part of the deposition process, and/or after the nanoscale wires have been deposited on the substrate. Thus, the alignment can occur before or after drying or other removal of the liquid, if a liquid is used. Any suitable technique may be used for alignment of the nanoscale wires. For example, the nanoscale wires can be aligned by passing or sliding substrates containing the nanoscale wires past each other (see, e.g., International Patent Application No. PCT/US2007/008540, filed Apr. 6, 2007, entitled “Nanoscale Wire Methods and Devices,” by Nam, et al., published as WO 2007/145701 on Dec. 21, 2007, incorporated herein by reference in its entirety), the nanoscale wires can be aligned using Langmuir-Blodgett techniques (see, e.g., U.S. patent application Ser. No. 10/995,075, filed Nov. 22, 2004, entitled “Nanoscale Arrays and Related Devices,” by Whang, et al., published as U.S. Patent Application Publication No. 2005/0253137 on Nov. 17, 2005, incorporated herein by reference in its entirety), the nanoscale wires can be aligned by incorporating the nanoscale wires in a liquid film or “bubble” which is deposited on the substrate (see, e.g., U.S. patent application Ser. No. 12/311,667, filed Apr. 8, 2009, entitled “Liquid Films Containing Nanostructured Materials,” by Lieber, et al., published as U.S. Patent Application Publication No. 2010/0143582 on Jun. 10, 2010, incorporated by reference herein in its entirety), or a gas or liquid can be passed across the nanoscale wires to align the nanoscale wires (see, e.g., U.S. Pat. No. 7,211,464, issued May 1, 2007, entitled “Doped Elongated Semiconductors, Growing Such Semiconductors, Devices Including Such Semiconductors, and Fabricating Such Devices,” by Lieber, et al.; and U.S. Pat. No. 7,301,199, issued Nov. 27, 2007, entitled “Nanoscale Wires and Related Devices,” by Lieber, et al., each incorporated herein by reference in its entirety). Combinations of these and/or other techniques can also be used in certain instances. In some cases, the gas may comprise an inert gas and/or a noble gas, such as nitrogen or argon.

In certain embodiments, a “lead” polymer is deposited (230 in FIG. 15), e.g., on the sacrificial material and/or on at least some of the nanoscale wires. The lead polymer may include one or more polymers, which may be deposited as one or more layers. The lead polymer can be used to cover or protect metal leads or other conductive pathways, which may be subsequently deposited on the lead polymer. In some embodiments, the lead polymer can be deposited, e.g., as a layer of material such that portions of the lead polymer can be subsequently removed, for instance, using lithographic techniques such as e-beam lithography, photolithography, X-ray lithography, extreme ultraviolet lithography, ion projection lithography, etc., or using other techniques for removing polymer that are known to those of ordinary skill in the art, similar to the bedding polymers previously discussed. However, the lead polymers need not be the same as the bedding polymers (although they can be), and they need not be deposited using the same techniques (although they can be). In some cases, more than one lead polymer may be used, e.g., deposited as more than one layer (for example, sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Any suitable polymer can be used as the lead polymer. In some cases, one or more of the polymers may be chosen to be biocompatible and/or biodegradable. For example, in one set of embodiments, one or more of the polymers may comprise poly(methyl methacrylate). In certain embodiments, one or more of the lead polymers comprises a photoresist, such as those described herein.

In certain embodiments, one or more of the lead polymers may be heated or baked, e.g., before or after depositing nanoscale wires thereon as discussed below, and/or before or after patterning the lead polymer. For example, such heating or baking, in some cases, is important to prepare the polymer for lithographic patterning. In various embodiments, the lead polymer may be heated to a temperature of at least about 30° C., at least about 65° C., at least about 95° C., at least about 150° C., or at least about 180° C., etc.

Next, a metal or other conductive material can be deposited (235 in FIG. 15), e.g., on one or more of the lead polymer, the sacrificial material, the nanoscale wires, etc. to form a metal lead or other conductive pathway. More than one metal can be used, which may be deposited as one or more layers. For example, a first metal may be deposited, e.g., on one or more of the lead polymers, and a second metal may be deposited on at least a portion of the first metal. Optionally, more metals can be used, e.g., a third metal may be deposited on at least a portion of the second metal, and the third metal may be the same or different from the first metal. In some cases, each metal may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, less than about 80 nm, less than about 60 nm, less than about 40 nm, less than about 30 nm, less than about 20 nm, less than about 10 nm, less than about 8 nm, less than about 6 nm, less than about 4 nm, or less than about 2 nm, etc., and the layers may be of the same or different thicknesses.

Any suitable technique can be used for depositing metals, and if more than one metal is used, the techniques for depositing each of the metals may independently be the same or different. For example, in one set of embodiments, deposition techniques such as sputtering can be used. Other examples include, but are not limited to, physical vapor deposition, vacuum deposition, chemical vapor deposition, cathodic arc deposition, evaporative deposition, e-beam PVD, pulsed laser deposition, ion-beam sputtering, reactive sputtering, ion-assisted deposition, high-target-utilization sputtering, high-power impulse magnetron sputtering, gas flow sputtering, or the like.

The metals can be chosen in some cases such that the deposition process yields a pre-stressed arrangement, e.g., due to atomic lattice mismatch, which causes the subsequent metal leads to warp or bend, for example, once released from the substrate. Although such processes were typically undesired in the prior art, in certain embodiments of the present invention, such pre-stressed arrangements may be used to cause the resulting device to form a 3-dimensional structure, in some cases spontaneously, upon release from the substrate. However, it should be understood that in other embodiments, the metals may not necessary be deposited in a pre-stressed arrangement.

Examples of metals that can be deposited (stressed or unstressed) include, but are not limited to, aluminum, gold, silver, copper, molybdenum, tantalum, titanium, nickel, tungsten, chromium, palladium, as well as any combinations of these and/or other metals. For example, a chromium/palladium/chromium deposition process, in some embodiments, may form a pre-stressed arrangement that is able to spontaneously form a 3-dimensional structure after release from the substrate.

In certain embodiments, a “coating” polymer can be deposited (240 in FIG. 15), e.g., on at least some of the conductive pathways and/or at least some of the nanoscale wires. The coating polymer may include one or more polymers, which may be deposited as one or more layers. In some embodiments, the coating polymer may be deposited on one or more portions of a substrate, e.g., as a layer of material such that portions of the coating polymer can be subsequently removed, e.g., using lithographic techniques such as e-beam lithography, photolithography, X-ray lithography, extreme ultraviolet lithography, ion projection lithography, etc., or using other techniques for removing polymer that are known to those of ordinary skill in the art, similar to the other polymers previously discussed. The coating polymers can be the same or different from the lead polymers and/or the bedding polymers. In some cases, more than one coating polymer may be used, e.g., deposited as more than one layer (e.g., sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Any suitable polymer may be used as the coating polymer. In some cases, one or more of the polymers can be chosen to be biocompatible and/or biodegradable. For example, in one set of embodiments, one or more of the polymers may comprise poly(methyl methacrylate). In certain embodiments, one or more of the coating polymers may comprise a photoresist, e.g., as discussed herein.

In certain embodiments, one or more of the coating polymers can be heated or baked, e.g., before or after depositing nanoscale wires thereon as discussed below, and/or before or after patterning the coating polymer. For example, such heating or baking, in some cases, is important to prepare the polymer for lithographic patterning. In various embodiments, the coating polymer may be heated to a temperature of at least about 30° C., at least about 65° C., at least about 95° C., at least about 150° C., or at least about 180° C., etc.

After formation of the device, some or all of the sacrificial material may then be removed in some cases. In one set of embodiments, for example, at least a portion of the sacrificial material is exposed to an etchant able to remove the sacrificial material. For example, if the sacrificial material is a metal such as nickel, a suitable etchant (for example, a metal etchant such as a nickel etchant, acetone, etc.) can be used to remove the sacrificial metal. Many such etchants may be readily obtained commercially. In addition, in some embodiments, the device can also be dried, e.g., in air (e.g., passively), by using a heat source, by using a critical point dryer, etc.

In certain embodiments, upon removal of the sacrificial material, pre-stressed portions of the device (e.g., metal leads containing dissimilar metals) can spontaneously cause the device to adopt a 3-dimensional structure. In some cases, the device may form a 3-dimensional structure as discussed herein. For example, the device may have an open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%. The device may also have, in some cases, an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm, and/or an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers, etc.

However, in other embodiments, further manipulation may be needed to cause the device to adopt a 3-dimensional structure, e.g., one with properties such as is discussed herein. For example, after removal of the sacrificial material, the device may need to be rolled, curled, folded, creased, etc., or otherwise manipulated to form the 3-dimensional structure. Such manipulations can be done using any suitable technique, e.g., manually, or using a machine. In some cases, the device, after insertion into matter, is able to expand, unroll, uncurl, etc., at least partially, e.g., due to the shape or structure of the device. For example, in FIG. 1B, a mesh device is able to expand after leaving the syringe.

Other materials may be also added to the device, e.g., before or after it forms a 3-dimensional structure, for example, to help stabilize the structure, to add additional agents to enhance its biocompatibility (e.g., growth hormones, extracellular matrix protein, Matrigel™, etc.), to cause it to form a suitable 3-dimension structure, to control pore sizes, etc. Non-limiting examples of such materials have been previously discussed above, and include other polymers, growth hormones, extracellular matrix protein, specific metabolites or nutrients, additional device materials, or the like. Many such growth hormones are commercially available, and may be readily selected by those of ordinary skill in the art based on the specific type of cell or tissue used or desired. Similarly, non-limiting examples of extracellular matrix proteins include gelatin, laminin, fibronectin, heparan sulfate, proteoglycans, entactin, hyaluronic acid, collagen, elastin, chondroitin sulfate, keratan sulfate, Matrigel™, or the like. Many such extracellular matrix proteins are available commercially, and also can be readily identified by those of ordinary skill in the art based on the specific type of cell or tissue used or desired.

In addition, the device can be interfaced in some embodiments with one or more electronics, e.g., an external electrical system such as a computer or a transmitter (for instance, a radio transmitter, a wireless transmitter, etc.). In some cases, electronic testing of the device may be performed, e.g., before or after implantation into a subject. For instance, one or more of the metal leads may be connected to an external electrical circuit, e.g., to electronically interrogate or otherwise determine the electronic state or one or more of the nanoscale wires within the device. Such determinations may be performed quantitatively and/or qualitatively, depending on the application, and can involve all, or only a subset, of the nanoscale wires contained within the device, e.g., as discussed herein. The connections may include, for example, anisotropic conductive films and/or surfaces having conductive inks, e.g., carbon nanotube inks.

The following documents are incorporated herein by reference in their entireties: U.S. Pat. No. 7,211,464, issued May 1, 2007, entitled “Doped Elongated Semiconductors, Growing Such Semiconductors, Devices Including Such Semiconductors, and Fabricating Such Devices,” by Lieber, et al.; and U.S. Pat. No. 7,301,199, issued Nov. 27, 2007, . 12/308,207, filed Ser. No. 10/588,833, filed Aug. 9, 2006, entitled “Nanostructures Containing Metal-Semiconductor Compounds,” by Lieber, et al., published as U.S. Patent Application Publication No. 2009/0004852 on Jan. 1, 2009; U.S. patent application Ser. No. 10/995,075, filed Nov. 22, 2004, entitled “Nanoscale Arrays, Robust Nanostructures, and Related Devices,” by Whang, et al., published as 2005/0253137 on Nov. 17, 2005; U.S. patent application Ser. No. 11/629,722, filed Dec. 15, 2006, entitled “Nanosensors,” by Wang, et al., published as U.S. Patent Application Publication No. 2007/0264623 on Nov. 15, 2007; International Patent Application No. PCT/US2007/008540, filed Apr. 6, 2007, entitled “Nanoscale Wire Methods and Devices,” by Lieber et al., published as WO 2007/145701 on Dec. 21, 2007; U.S. Patent Application Serial No Dec. 9, 2008, entitled “Nanosensors and Related Technologies,” by Lieber, et al.; U.S. Pat. No. 8,232,584, issued Jul. 31, 2012, entitled “Nanoscale Sensors,” by Lieber, et al.; U.S. patent application Ser. No. 12/312,740, filed May 22, 2009, entitled “High-Sensitivity Nanoscale Wire Sensors,” by Lieber, et al., published as U.S. Patent Application Publication No. 2010/0152057 on Jun. 17, 2010; International Patent Application No. PCT/US2010/050199, filed Sep. 24, 2010, entitled “Bent Nanowires and Related Probing of Species,” by Tian, et al., published as WO 2011/038228 on Mar. 31, 2011; U.S. patent application Ser. No. 14/018,075, filed Sep. 4, 2013, entitled “Methods And Systems For Scaffolds Comprising Nanoelectronic Components,” by Lieber, et al.; and Int. Patent Application Serial No. PCT/US2013/055910, filed Aug. 19, 2013, entitled “Nanoscale Wire Probes,” by Lieber, et al.

In addition, U.S. Patent Application Serial No. 14/018,075, filed Sep. 4, 2014, entitled “Methods And Systems For Scaffolds Comprising Nanoelectronic Components,” by Lieber, et al., published as U.S. Patent Application Publication No. 2014/0073063 on Mar. 13, 2014; U.S. patent application Ser. No. 14/018,082, filed Sep. 4, 2013, entitled “Scaffolds Comprising Nanoelectronic Components For Cells, Tissues, And Other Applications,” by Lieber, et al., published as U.S. Patent Application Publication No. 2014/0074253 on Mar. 13, 2014; International Patent Application No. PCT/US14/32743, filed Apr. 2, 2014, entitled “Three-Dimensional Networks Comprising Nanoelectronics,” by Lieber, et al.; or U.S. Provisional Patent Application Ser. No. 61/911,294, filed Dec. 3, 2013, entitled “Nanoscale Wire Probes for the Brain and other Applications,” by Lieber, et al. are each incorporated herein by reference in its entirety.

Furthermore, U.S. Provisional Patent Application Ser. No. 61/975,601, filed Apr. 4, 2014, entitled “Systems and Methods for Injectable Devices” is incorporated herein by reference in its entirety.

The following examples are intended to illustrate certain embodiments of the present invention, but do not exemplify the full scope of the invention.

### EXAMPLE 1

Recent advancements in electronics fabrication include the fabrication of electronics on flexible, stretchable and 3D substrates so that electronics can cover soft or non-planar surfaces. New requirements have risen for implementing electronics into objects with minimal invasiveness, 3D distributing nano- and micro-scale sensor units with microscale spatio-resolution in a large volume and mechanically ultra-flexibility.

Some examples have shown that either using a substrate to deliver electronics into biological samples with subsequently being released from substrate or constructing a 3D network of the electronics, as discussed in U.S. patent application Ser. No. 14/018,075, filed Sep. 4, 2014, entitled “Methods And Systems For Scaffolds Comprising Nanoelectronic Components,” by Lieber, et al., published as U.S. Patent Application Publication No. 2014/0073063 on Mar. 13, 2014; U.S. patent application Ser. No. 14/018,082, filed Sep. 4, 2013, entitled “Scaffolds Comprising Nanoelectronic Components For Cells, Tissues, And Other Applications,” by Lieber, et al., published as U.S. Patent Application Publication No. 2014/0074253 on Mar. 13, 2014; International patent application Ser. No. PCT/US14/32743, filed Apr. 2, 2014, entitled “Three-Dimensional Networks Comprising Nanoelectronics,” by Lieber, et al.; or U.S. Provisional Patent Application Ser. No. 61/911,294, filed Dec. 3, 2013, entitled “Nanoscale Wire Probes for the Brain and other Applications,” by Lieber, et al., each incorporated herein by reference in its entirety. In addition, the development of soft materials (gel, fibers, etc.) and freestanding nano- or bio-materials (microbeads, viral vectors, etc) brings many examples of materials that have large porosities, freestanding and small volume, which can be syringe-injected/delivered with vanishingly little invasiveness into the target system followed by fully integration into the target system. However, nanowires have not typically been incorporated in such materials.

This example describes a strategy for electronics design using nanowires. This strategy involves encapsulating electronics units into a mesh polymeric network that mimics the structure of soft materials and freestanding nanomaterials. In this study, silicon nanowires were used as semiconductor components and metal electrodes were used as electrical sensing units given their nano- and micro-scale structure, multifunctionalities and electrical and chemical recording capability.

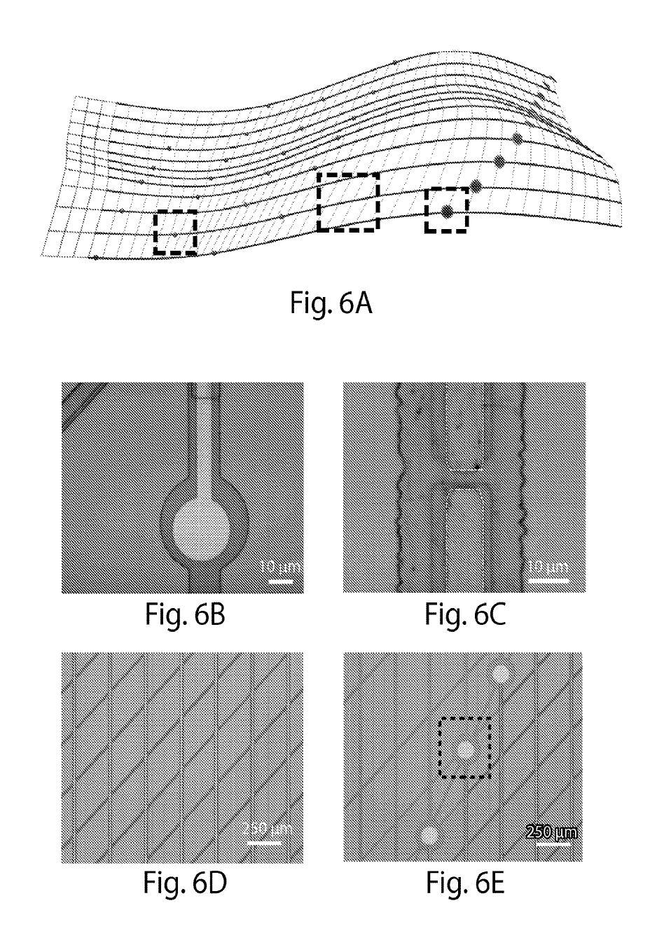

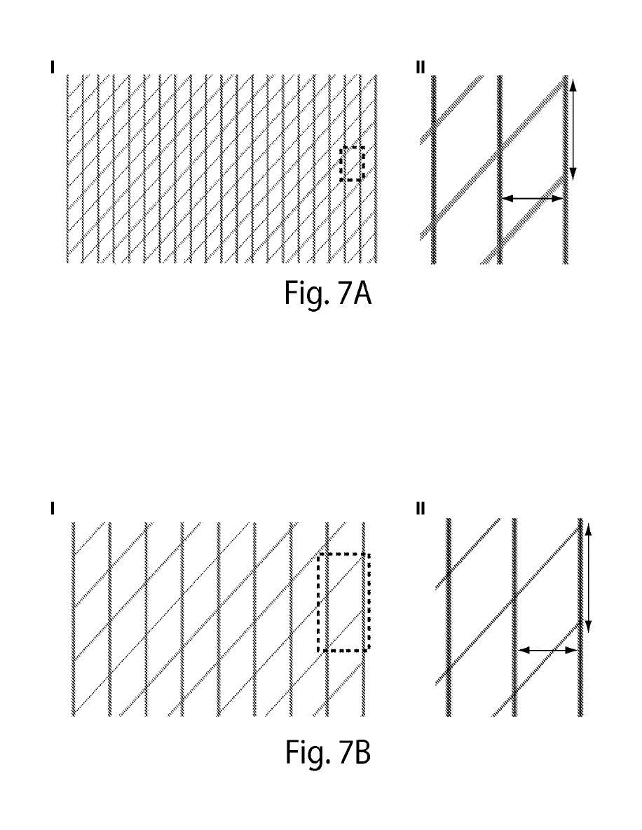

FIGS. 1A and 1B show schematics of the basic idea of this example. The electronics are in a mesh network encapsulated in photodefinable epoxy (SU-8), which is fabricated on a nickel sacrificial layer (see, e.g., International patent application Ser. No. PCT/US14/32743, filed Apr. 2, 2014, entitled “Three-Dimensional Networks Comprising Nanoelectronics,” by Lieber, et al., incorporated herein by reference), and then completely removed from the substrate with electronic sensor units, metal connections and input/output (I/O) pads all distributed on this freestanding network (FIGS. 1A and 6). Electronics were loaded into syringe and then delivered/injected through the needle (FIG. 1B) with its subsequent geometrical restoration. In this design, the width of ribbons in the network was typically 5 to 40 micrometers, the total thickness was less than 800 nm, and size of unit cells was several hundred micrometers (FIG. 7). FIG. 1C shows a 3D reconstructed confocal fluorescence image of a representative injection. 2-mm-wide syringe injectable electronics were injected through a glass needle with 95-micrometer inner diameters into aqueous phosphate buffer saline (PBS). This electronics had ribbons with feature sizes of 5 micrometers and thicknesses of 700 to 800 nm. The surface of the electronics was modified by poly-D-lysine (0.5-1.0 mg/mL, MW 70,000 to 150,000) to make the surface of the electronics hydrophilic, allowing the electronics to be suspended in PBS solution.

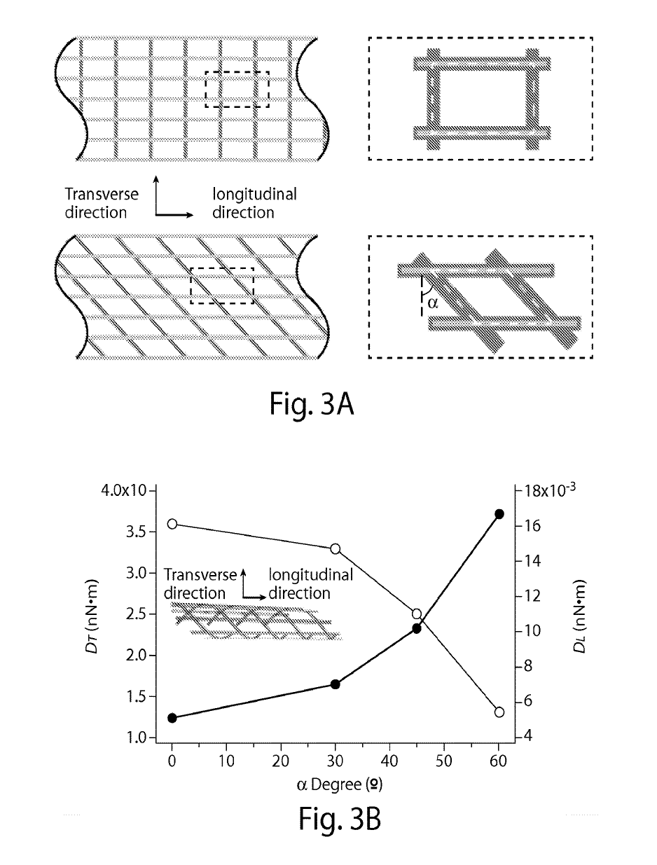

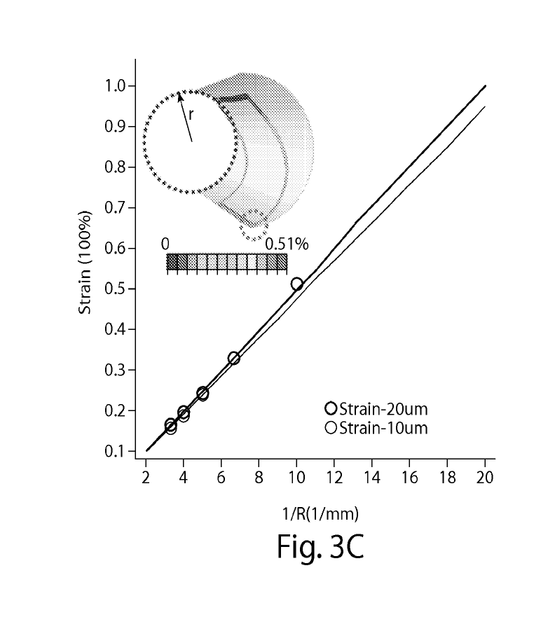

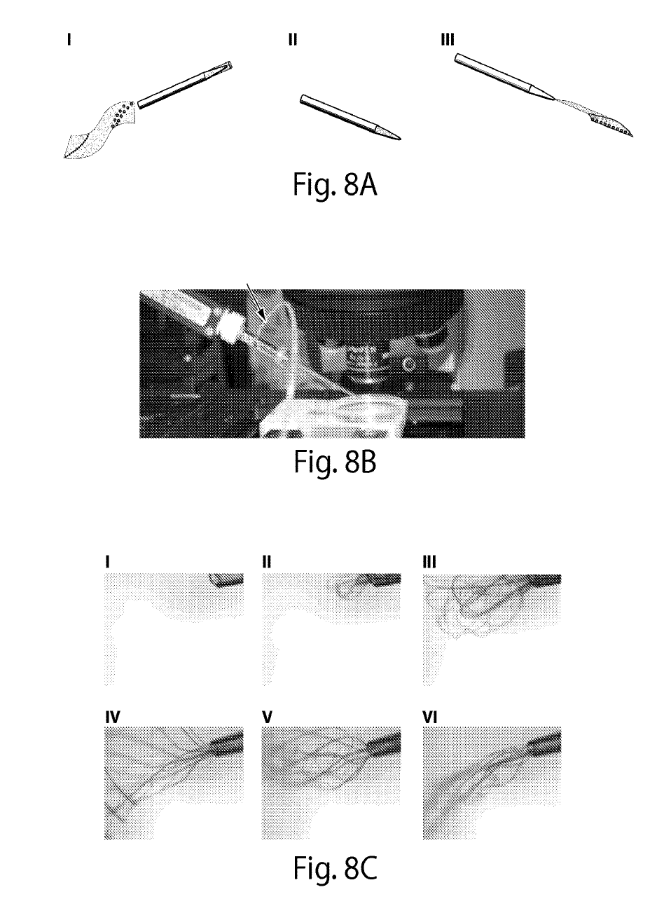

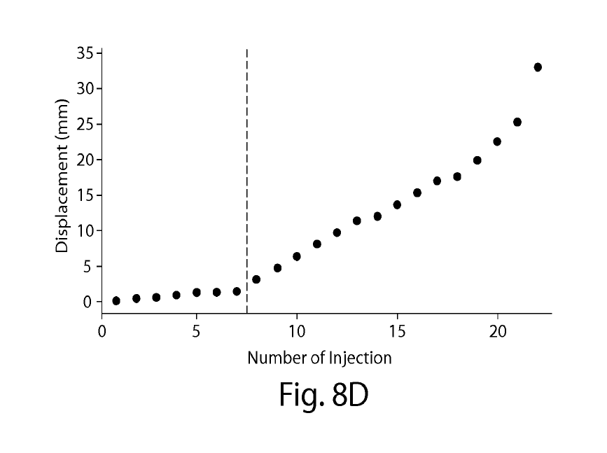

The stepwise process of this injection into free solution is shown in FIG. 1D. The electronics were loaded into a glass tube (with a 95 micrometer tip) by first connecting the glass needle to a syringe via a plastic tube, and drawing into the end of glass needle (FIG. 8A). The glass needle was then detached from the syringe and mounted onto a commercially available patch-clamp system (FIG. 3B). A microinjector (ALA Scientific Instruments) or the manually controlled syringe was connected to the glass needle to apply sufficient pressure (1 bar, 1-10 ms) for injection. Using the microinjector, the electronics could be injected out gradually from needle with the electronics being displaced from the needle by 5 to 10 micrometers per injection, with less than 100 nL solution in each injection (FIG. 3C). The injected region of electronics gradually unfolded in solution to reduce the surface energy and internal strain.

The injection process could be controlled such that only the region of electronics containing the nanodevices was injected into the target system whereas the metal contact and I/O region were injected outside for external control. Anisotropic conductive film (ACF) was used in some experiments to bond the I/O pads of electronics with external set-ups (FIGS. 1E and 1F). However, in other embodiments, other systems, such as carbon nanotubes, may be used for the connections, e.g., as discussed below. The relationship between the yield of injection and the electrical performance of electronics to the inner diameter of needle was evaluated by injection through conventional metal gauge needle in PBS solution. The average yield of injection for nanowire electronics ranged from 98% with needle diameters larger than 600 micrometers to 83% with needle diameters of 100 micrometers. Less than a 12% conductance change in average was observed after injection.

FIG. 1 shows various syringe injectable electronics. FIG. 1A is a schematic of the electronics design in mesh structure for injection. FIG. 1B is a schematic injection process. The metal contacts and input/output (I/O) pads are in black. The needle tip, highlighted from the syringe, is shown by dashed circle. FIG. 1C is a 3D-reconstructed fluorescence image showing that after the electronics were injected out of the needle, the electronics subsequently unfolded by itself in the solution. FIG. 1D are images showing that the electronics were stepwise injected into solution by a glass needle with diameter of 95 micrometers. The electronics have been pushed to the tip of needle (I), the electronics were partially injected out (II), 50% of total surface area of the electronics has been injected out, with partially unfolded mesh structure remaining near needle region (III) corresponding to the region highlighted by dashed box in FIG. 1C, and the completely unfolded mesh structure (IV) corresponding to the region was highlighted by a white dashed box in FIG. 1C. FIG. 1E is a schematic of bonding. (I) shows a flexible cable, (II) shows anisotropic conductive film (ACF), and (III) shows unfolded I/O region of electronics on substrate (IV). FIG. 1F is an optical micrograph showing that the electronics were fully injected into chamber with solution and fully unfolded. The I/O region was dried and bonded by ACF to flexible cable for measurement. FIG. 1G shows yield (upper graph) and conductance change (lower graph) of nanowire electronics injected through different gauge needles with two kinds of mesh shown in FIG. 7

FIG. 6 shows optical images of the device structure. FIG. 6A is a schematic of the syringe injectable electronics used in these examples, including metal contact and I/O pads, supporting polymer and the device. FIG. 6B is an optical micrograph of device region on the mesh corresponds to right dashed box in FIG. 6A. FIG. 6C is an SEM image of the nanowire device corresponds to the middle dashed box in FIG. 6A. FIGS. 6D and 6E are optical images of the device.

FIG. 7 shows design of the meshes used in these particular examples; other meshes or configurations are also possible. FIG. 7A shows the structure of mesh for injection through needle with inner diameter larger than 200 micrometers, including SU-8 and metal. (I) is a schematic of the whole mesh; the width is 5 to 15 mm. (II) is a zoomed-in region indicated by black dashed box in (I), highlighting a single unit cell. The length of unit cell is 333 micrometers and width of unit cell is 250 micrometers. The width of ribbon is 20 micrometers. FIG. 7B shows the structure of mesh for injection through a needle smaller than 100 micrometers. (I) shows a schematic of the whole mesh; the width is 2 mm. (II) shows a zoomed-in region indicated by black dashed box in (I), highlighting a single unit cell. The length of unit cell is 333 micrometers and width of unit cell is 250 micrometers. The width of transverse ribbon and longitudinal ribbon is 10 micrometers and 20 micrometers, respectively.

Altogether, these results demonstrate the success of the injection of mesh electronics without hindering the integrity and performance of the electronics. Several design factors were considered, including: (1) the nanometer-thickness and mesh design increased the surface-to-volume ratio of electronics from 0.2 micrometer⁻¹ (micrometer²/micrometer³) for typical 10-micrometer-thick thin film electronics to 3.25 micrometers⁻¹ for a 5-micrometer-wide ribbon mesh electronics, (a) with polyelectrolyte surface charge modifications, reducing the effective density of electronics (due to the forming of electric double layers) making electronics suspended in solution, and (b) increasing the drag force of solution motion to electronics, allowing the electronics to be readily displaced by solution motion for injection; (2) the nanometer-thickness and mesh layout together (a) reduced the effective bending stiffness of electronics from 0.0602 nN·m for a thin-film electronics with the same thickness, to 0.0025 nN·m for the mesh electronics so that it could be readily bent and injected into needles, (b) reduced the strain during injection, and (c) reduced the total volume of the electronics to allow the electronics to go through a small diameter needle.

### EXAMPLE 2

To further understand the structure design parameters for injection, imaging experiments were performed in this example using confocal fluorescence imaging to 3D reconstruct the structure of injectable electronics inside the glass needle. A glass tube was pulled into a fluidic channel (FIG. 2A), with the same geometry and inner diameter as the metal and glass needle used for applications, which allowed the electronics to be injected through for imaging. The channel inner diameter was 200 to 600 micrometers, as measured by confocal fluorescence imaging, and the length was 0.1 to 0.5 mm. Electronics with different structures were injected through the tube region into channel by syringe. SU-8 of electronics was doped by Rodamine-6G for imaging and 3D reconstruction for analysis.

The ribbons along the injection direction were called longitudinal ribbons and the ribbons perpendicular to the injection direction were called transverse ribbons. Longitudinal and transverse ribbons together form a mesh with a periodic unit cells structure. All the unit cells were identical in this experiment. Metal connections and nanodevices were mainly encapsulated in the longitudinal ribbons (FIG. 2B). The ribbons in the design for imaging experiments were 20-micrometer-wide and 700-nm-thick for SU-8 and 10-micrometer-wide and 100-nm-thick for metal. Different widths of mesh were used for investigation because wider electronics may allow sensing units to cover a larger area. The meshes used here had a sharp tip of 45° , which allowed them to be loaded into needles at the same tip point (FIG. 2B).

Two different meshes with different unit cell geometries have been used here to investigate the injection. In design #1 (FIG. 2B, I), the transverse ribbons were tilted 45° counterclockwise in transverse direction on the mesh plane forming a 45° angle to longitudinal ribbons. In design #2 (FIG. 2B, II), the transverse ribbons were perpendicular to the longitudinal ribbons to form an orthogonal mesh. FIGS. 2C-2E shows optical micrographs, 3D-reconstructed and cross-section images of assembled structures for each mesh in the glass channel.

Firstly, 5-mm-wide mesh with the #1 design structure were smoothly injected through a channel with ca. 500-micrometer-inner-diameter (FIG. 2C, I). The 3D-reconstructed image shows that the mesh had been rolling into a tubular structure inside the channel, which kept the longitudinal ribbons straight and made the transverse ribbon bent (FIG. 2D, I). The cross-section image of the 3D reconstruction further confirmed the tubular structure, illustrating that the ribbons were closely and uniformly packed close to the inner surface of glass channel. The other half of mesh in the bottom part of the needle was blocked from imaging by the dense ribbons on the top part of channel. Imaging of a larger channel (>600 micrometers, inner diameter) injected with mesh for a more sparse ribbon density showed a full tubular structure (FIG. 10B).

Secondly, reducing the channel's inner diameter did not affect the assembled structure of mesh in the needle. The same mesh could be injected smoothly through 200-micrometer-inner-diameter channel (FIG. 2C, II). The 3D-reconstructed and cross-section images further demonstrated the tubular structure of mesh in the needle and closed packed ribbons to the inner wall of channel (FIGS. 2D, II and 2E, II).

Thirdly, increasing the width of mesh can also allow the mesh to be smoothly injected through channels. As a representative example, 15-mm-wide mesh was injected through the channel with an inner diameter of ca. 500 micrometers (FIG. 2C, III). The width-to-inner-diameter ratio was ca. 30. The 3D-reconstructed and cross-section images (FIG. 2D, III and 2E, III) showed the tubular structure of mesh in the channel and closed packed ribbons to the inner wall of channel. Although the density of ribbon had been greatly increased, the longitudinal ribbons still remained straight during injection.

Fourthly, as the control sample, it was found that the mesh with the #2 design could not be as easily injected through the channel with 500-micrometer inner diameter. Differences with the regularly tubular structure formed by design #1, a 10-mm-wide mesh sometimes formed a jammed structure caused by ribbon entanglement, blocking the channel (FIG. 2C, IV). 3D-reconstructed and cross-section imaging further showed the ribbons entangled together in “buckles” (FIG. 2D, IV and 2E, IV), which filled the whole channel.

FIG. 2 shows parameters for injection, according to some embodiments. FIG. 2A is a schematic showing the structure of the pulled glass tube for testing and imaging the structure of different electronics designs in the needle. The arrow indicates the direction of injection. FIG. 2B are schematics of two different designs for injection. (I) shows a mesh with a 45° tilted transverse ribbon, and (II) shows a mesh with straight transverse ribbons. The dashed black circles highlight the detailed structure, with supporting and passivation polymer, and metal lines. FIG. 2C are optical images of different electronics designs injected through glass needle. (I-II) show 5-mm-wide meshes as designed in (FIG. 2B, I) were injected through 500-micrometer and 250-micrometer ID glass needles; (III) shows a 15-mm-wide mesh as designed in (FIG. 2B, I) was injected through a 450-micrometer . ID needle; (IV) shows a 10-mm mesh as design in (FIG. 2B, II) that was injected through a 450-micrometer ID needle. FIG. 2D shows a top view of a 3D reconstructed confocal images corresponding to FIG. 2C. FIG. 2E shows images at cross-sections as indicated by white dashed lines in FIG. 2C. White dashed curves in FIG. 2E highlights the cross-section of needle boundary.

In some cases, it may be important to keep the longitudinal ribbons straight during injection to avoid or at least minimize (1) the high-strain deformation to the electronics, which may damage the device and (2) buckling of the longitudinal ribbons. Buckling of the longitudinal ribbons can dramatically decrease the stiffness of the structure in the longitudinal direction, and therefore, casing collapse of the longitudinal ribbons, rather than bending of the transverse ribbons, causing large strain and damages of the device and even blocking the needle for further injection.

### EXAMPLE 3

Different assembly structure of these two meshes in the channel and needle may be understood as follows. The bending stiffness for the mesh bent in longitudinal direction and transverse direction of injection may be defined as D_{L} and D_{T}, respectively. Firstly, the orthogonal-transverse-ribbon design (design #2) lead to a non-uniform distribution of effective bending stiffness D_{L}. Considering the effective bending stiffness D_{L} of different cross-sections, when the cross-section goes through the transverse ribbons, the bending stiffness was high (0.0602 nN·m), while when the cross-section did not go through the transverse ribbons, the bending stiffness was lower (0.0025 nN·m). This dramatic bending stiffness change facilitates stress localization leading to the buckling of longitudinal ribbons. Tilted-transverse-ribbon designs (design #1) created a uniform distribution of effective bending stiffness D_{L}; therefore, the electronics could bend more homogeneously.

Secondly, this tilted-transverse-ribbon design decreases D_{T} and increase D_{L} so that the mesh was more readily to bend and roll-up into a tubular structure for going through the needle; the desgin was less readily buckle in the longitudinal direction.

Finite element modeling (FEM) analysis was also used to simulate the bending stiffness for mesh bending in two directions. Notably, reducing D_{T} and increasing D_{L} was beneficial to the injection process. FIG. 3A is a schematic showing selection of unit cells from the periodic mesh structure for an example simulation. The relation of angle a (alpha) that was between transverse ribbon and longitudinal ribbon to bending stiffness was investigated. The white dashed lines indicate the boundary for unit cells from mesh for simulation. The effective bending stiffness of mesh was defined as the stiffness required a homogenous beam to achieve the same bending under the same moment. Therefore, every unit cell had the same bending stiffness, and a unit cell was used to calculate the effective bending stiffness of the structure from the simulations.

These results (FIG. 3B) showed that by increasing a (alpha) from 0 to 60° , D_{T} decreased from 0.0036 to 0.0013 nN·m and D_{L} increased from 0.0051 to 0.0167 nN·m. The bending stiffness ratio between bending in transverse and longitudinal direction increased by about 8.7 times (1.46 to 12.8). Altogether, these results show that increasing the tilt angle may significantly facilitate the rolling of electronics in the needle in transverse direction to form a tubular structure, and/or prevent bending in the longitudinal direction that could lead to buckling and compression on same injection condition.

FIG. 3 shows mechanical analysis for an injection process according to one embodiment. FIG. 3A are schematics show the structures of two different mesh designs. Black dashed boxes highlight the unit cell structure, including supporting and passivation polymer and metal lines. FIG. 3B shows bending stiffness in the longitudinal (D_{L}) and transverse directions (D_{T}) of the mesh with the changes of ribbon angle in FIG. 3A. The inset is a schematic showing that the mesh rolled up in transverse direction in needle. FIG. 3C shows simulated highest strain value as functions of 1/r, with two kinds of mesh shown in FIG. 7. The inset is a representative simulation shows the strain distribution of unit cell in 200-micrometer ID needles. Smaller dashed circle highlights the point with highest strain. Larger dashed circle and black arrow show the inner boundary and diameter of the needle.

### EXAMPLE 4

This example uses simulations to estimate the strain distribution in the electronics during injections in needles with different sizes. Since every unit cell behaves similarly, the bending of only one unit cell to the curvature of the needle was simulated. The inset of FIG. 3C shows one typical unit cell bending structure inside 200-micrometer diameter needle, and shading indicates a contour plot of the maximal principle strain. The maximal value was reached on the junction between the transverse and longitudinal ribbons. Simulation results (FIG. 3C) showed the dependence of the maximal principal strain of the unit cell on the curvature of the needles 1/r, and a linear relation fitted to the dependence.

The colors correspond to two different sizes of the mesh structures (FIG. 7) used for needle inner diameter larger or smaller than 200 micrometers. The two corresponding fitting relation were 0.499/r and 0.473/r. For needle diameters around 100 micrometers, the maximal principle strain could be extrapolated as 0.998% and 0.946% respectively, which are both smaller than the critical breaking strain of SU-8 for bulk materials. The stress intensity factor K for a thin film under pure bending has the following scaling relation:

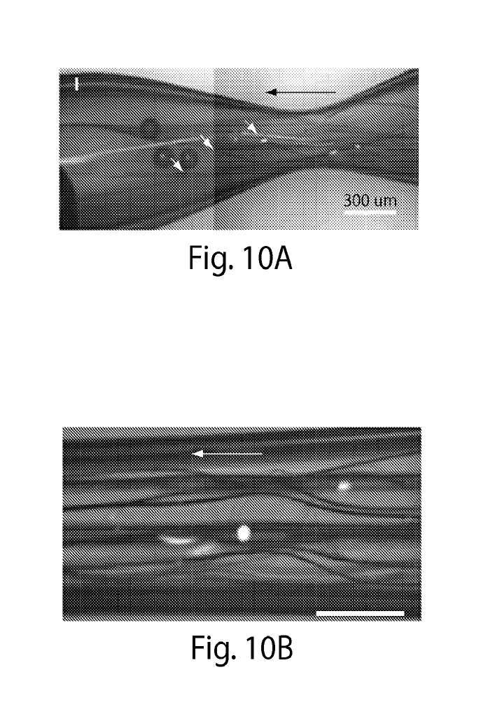

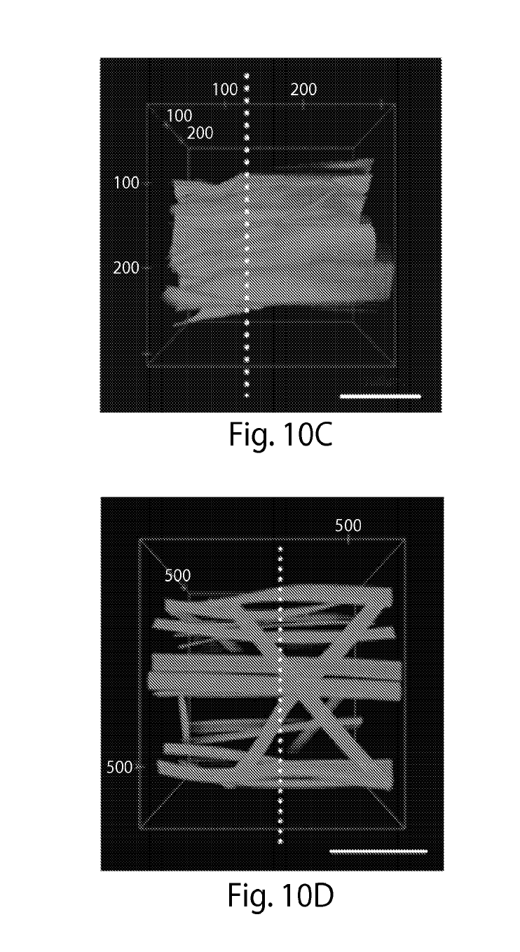

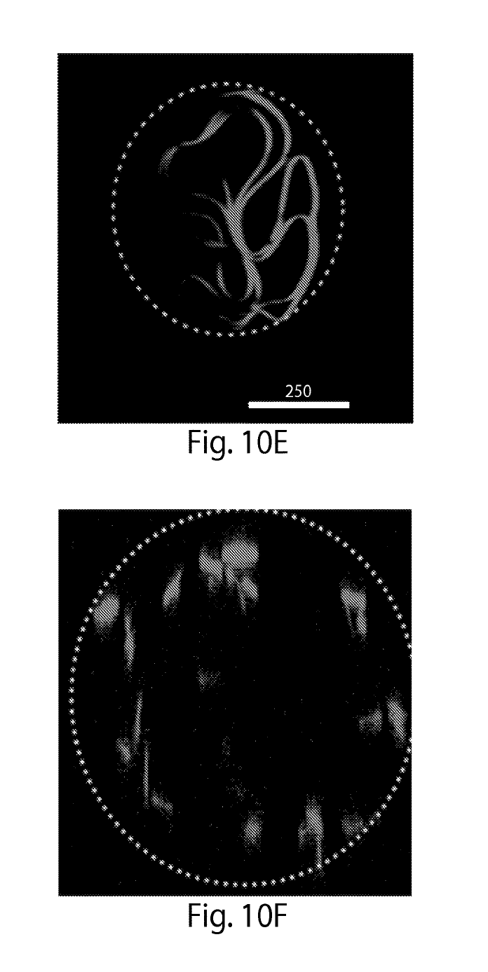

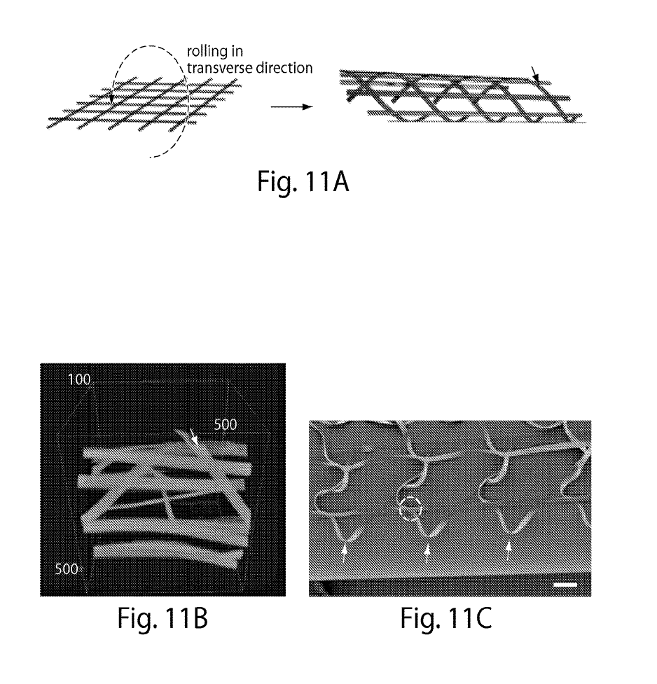

K˜Eε\sqrt{h},

where E is the Young's modulus of the material, and h is the thickness of ribbon. The ribbon may break when K reaches the toughness of the material K_{c}. K_{c} is usually on the order of 100 KPa\sqrt{m}, and E for SU-8 is around 1 GPa. Therefore, for a device with thickness of several hundred nanometers, the fracture strain e can be estimated to be on the order of several percent. In fact, with this current structure, experiment demonstrated that SU-8 ribbon can sustain the bending with curvature larger than 0.1 micrometers⁻¹, corresponding to the curvature of a 20-micrometer-diameter needle (FIG. 11).

FIG. 11 shows the mechanics of the mesh during rolling. FIG. 11A is a schematic showing that the mesh rolls up in a transverse direction in the needle. FIG. 11B shows a 3D-reconstructed fluorescence image of mesh rolling in needle with a 600-micrometer inner diameter. Arrow indicates the bending of transverse ribbons. FIG. 11C is an SEM image of the mesh folding on the substrate with the extreme twisting of transverse ribbons (arrows) and junctions (dashed circle). Scale bar: 100 micrometers.

### EXAMPLE 5

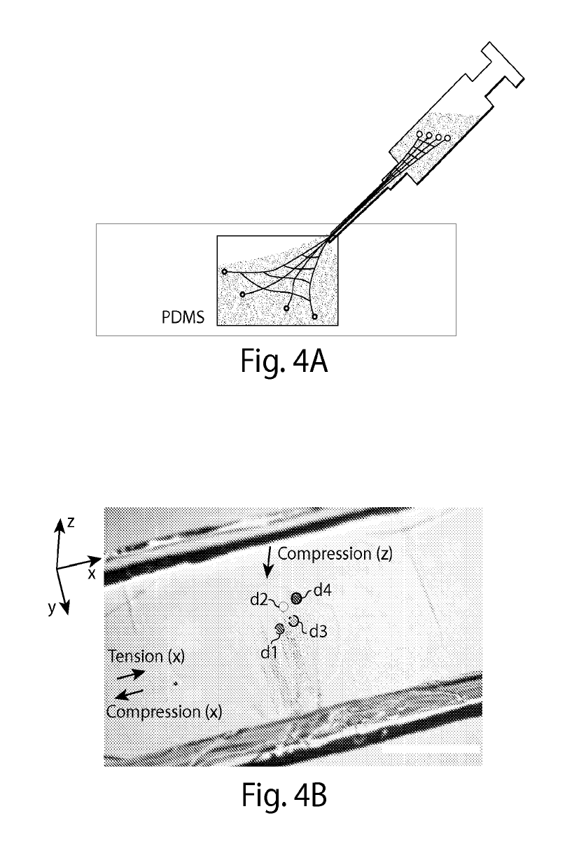

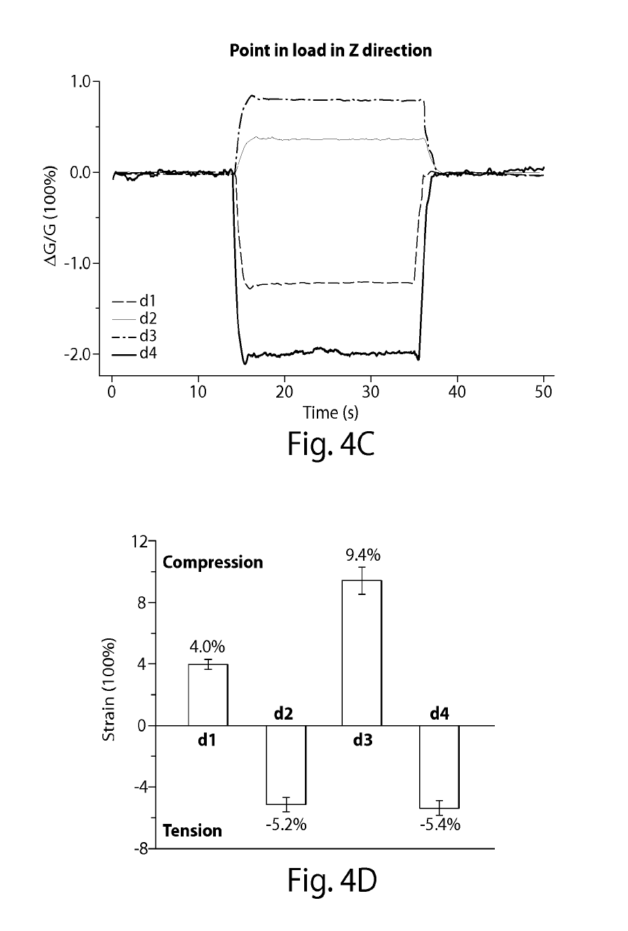

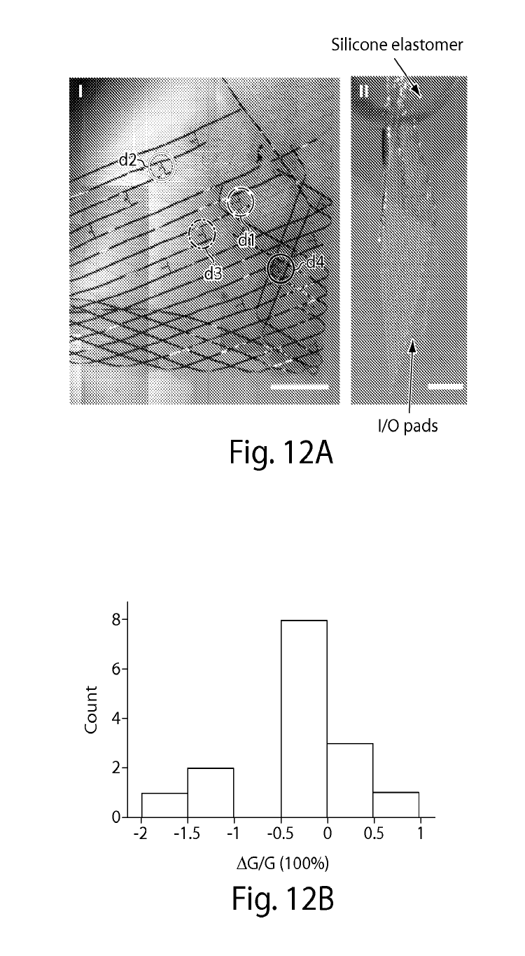

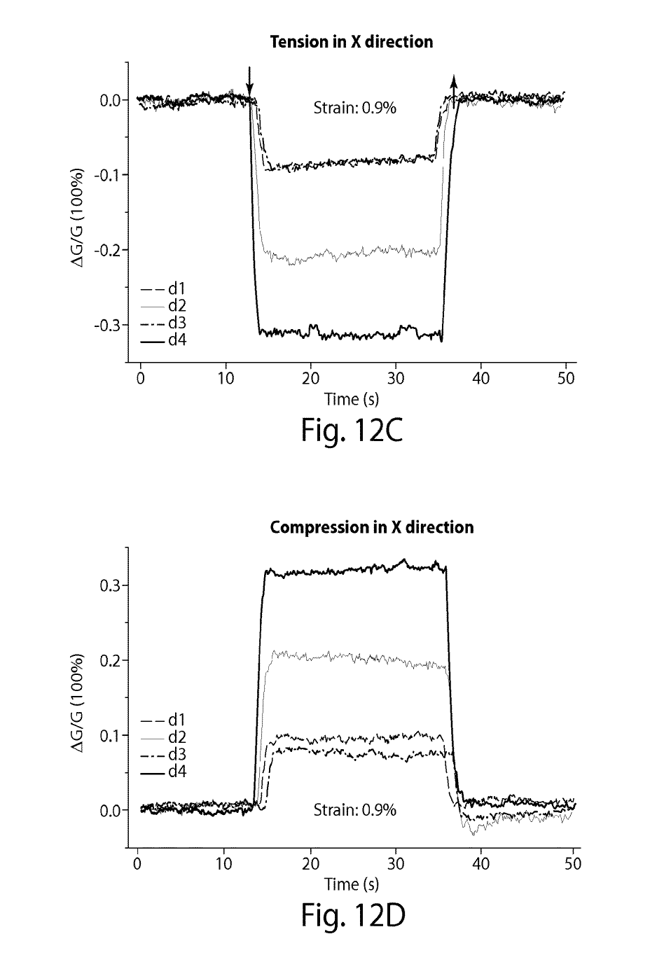

This example demonstrates that syringe injectable electronics could be injected with various mediums and materials into a cavity through a small injection site with subsequent geometrical restoration, allowing the electronic sensor unit to cover a much larger area within the cavity (FIG. 4A). 5-mm-wide electronics containing nanowire strain sensors were mixed with pre-cured poly-dimethylsiloxane (PDMS) (Dow Corp., Midland, Mich., USA), diluted in hexane, and then injected through a 20-gauge (603 micrometer) needle into a cavity constituted by two pieces of cured PDMS with the connections injected outside for bonding. The electronics within the cavity gradually unfolded, with the nanowire nanodevices fully separating from each other and covering a 5 mm×7 mm area. Four typical nanowire devices (d1, d2, d3, and d4) with the widest separation, as located in FIG. 4B, were used as multiple strain sensors. A uniform tensile strain of 0.9% along x direction (FIG. 4B) caused a decrease of conductance up to 0.34% (FIG. 12) for nanowire devices d1-d4. When a compressive strain with same value in x direction was applied, the conductance change had similar value but with opposite sign (FIG. 12D), which confirms the conductance change comes from strain deformation.

A point load in the z direction (FIG. 4B), introducing a non-uniform strain distribution in PDMS, is applied at the position elucidated in FIG. 4B, causing the conductance change in FIG. 4C. Compared to the results in FIG. 12A and 12B, the devices were under two kinds of strains (compressive: d1 and d3; tensile: d2 and d4) in this case. A calibrated conductance change of 0.9% strain value (FIG. 12C and 12D) was used to obtain localized strains when the hybrid structure was under point load in z direction, shown in FIG. 4D. This result shows that injected electronics can be used to measure strain distribution inside PDMS to the external mechanical deformation and demonstrate the injectable electronics can be used in materials and tissue interrogation with little damage to the target system.

FIG. 4 shows syringe injectable electronics for soft material. FIG. 4A is a schematic of co-injection of devices with PDMS precursor into cavity formed by cured PDMS. The electronics have been dissolved or suspended in PDMS/hexane (v/v=1:3) and injected into the cavity formed by two pieces of cured PDMS. FIG. 4B shows an optical image of devices after being injected and cured in PDMS. The arrows indicate the direction of the force applied in pizoresistive measurements (c and d), or outlines of nanowire device #d1, d2, d3 and d4 in FIG. 4C and 4D. The downward and upward arrows denote the times when the strain was applied and released, respectively. Scale bar: 1 cm. FIG. 4C shows real-time recording of conductance changes by multiplex devices located in FIG. 4B, under point load in z direction with deformation on PDMS. FIG. 4D shows the calculated strain localized near nanowire device #d1, d2, d3 and d4.

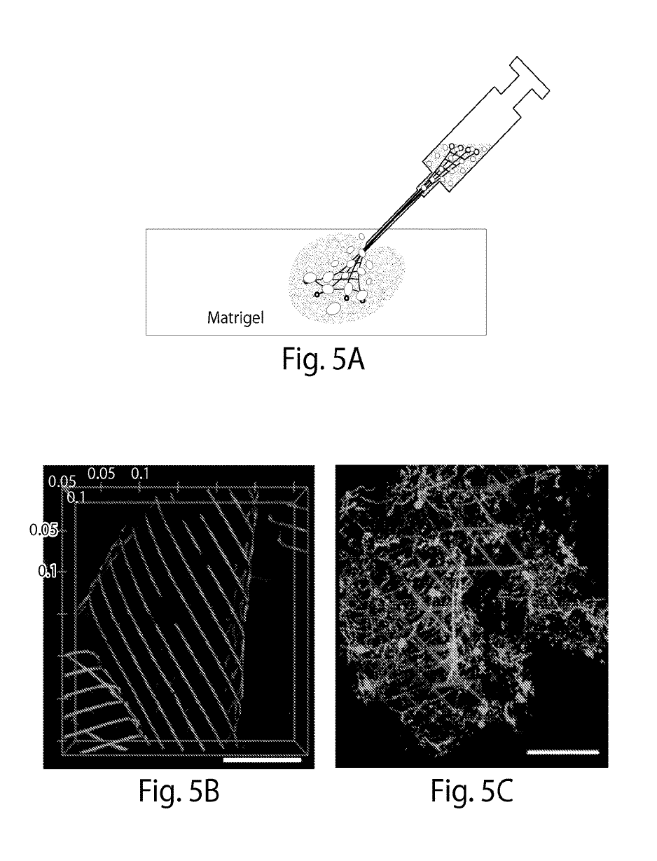

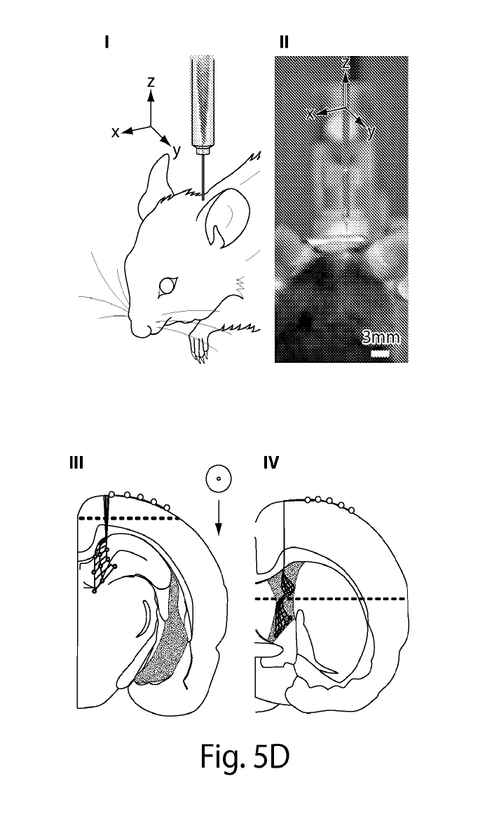

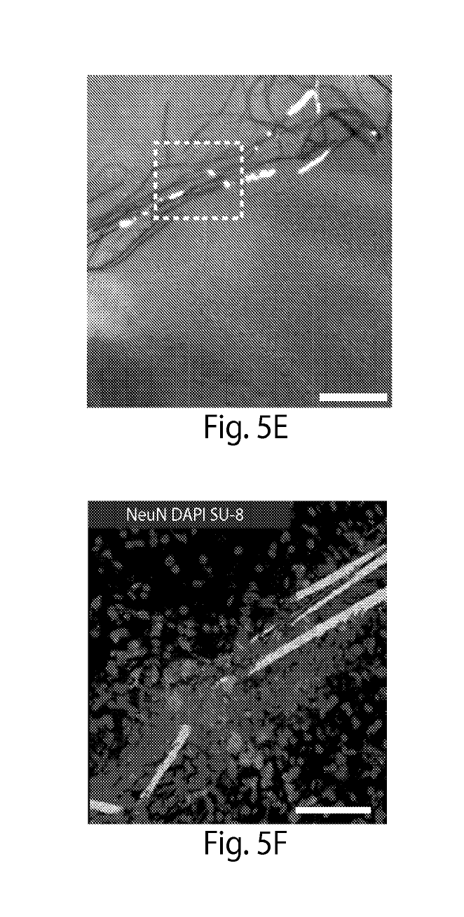

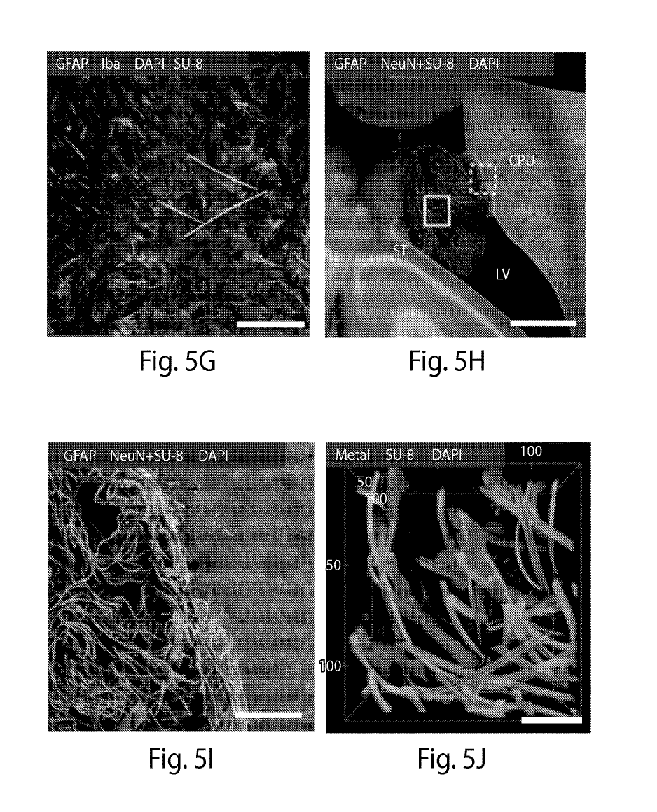

FIG. 12 shows injection of the electronics in soft matter. FIG. 11A shows optical images of injectable electronics in PDMS. (I) is a zoomed-in region of electronics in FIG. 4, where the circles outline nanowire device #1, 2, 3 and 4 measured in FIG. 4B. Scale bar: 1 mm. (II) shows that the I/O regions of the device in (I) unfolded and dried on substrate, with the transition part of device from PDMS to substrate fixed by silicone elastomer. Scale bar: 0.2 cm. FIG. 12B is a histogram of conductance changes of the nanowire devices in FIG. 12A, under point load in the z direction. FIGS. 12C and 12D are real-time recording of conductance changes by multiplex devices located in FIG. 12A, under uniform 5 mm tensile deformation (strain: 0.9%) in the x direction (FIG. 12C) and 5 mm compressive deformation (strain: 0.9%) in the x direction (FIG. 12D). The downward and upward arrows denote the times when the strain was applied and released, respectively.

The injection in continuous soft materials, especially biomaterials, was also tested (FIG. 10A). Specifically, 2-mm-wide electronics were injected into a 30% polymerized Matrigel through a 22-gauge (413 micrometer) needle, which first showed a compressed structure but unfolded 100% after incubation in 37° C. for 72 hours to allow the nanowire or metal electrode sensing unit to distribute in Matrigel (FIG. 5B). Moreover, the nature of the injectable electronics allowed for the injection with other biomaterials and even isolated cells. Embryonic rat hippocampal neurons were mixed with electronics and uncured Matrigel and subsequently injected into polymerized Matrigel (FIG. 5C). 3D-reconstructed confocal micrographs from two-week cultures showed that neurons with high-density outgrowth neurites interpenetrating in the mesh structure of electronics, proving the biocompatibility of the electronics. It is noticeable that the width of ribbons was similar to the neurite projections, exhibiting seamless integration between them.

Based on this results, the D_{L} of this injectable electronics was estimated to be ca. 0.01 nN m, which was similar to the bending stiffness of tissue and its bending energy matches the surface membrane energy and significantly less than the stiffness of a conventional silicon probe. The design of macroporous structures may also allow for the growth of tissue within the interior space.

### EXAMPLE 6

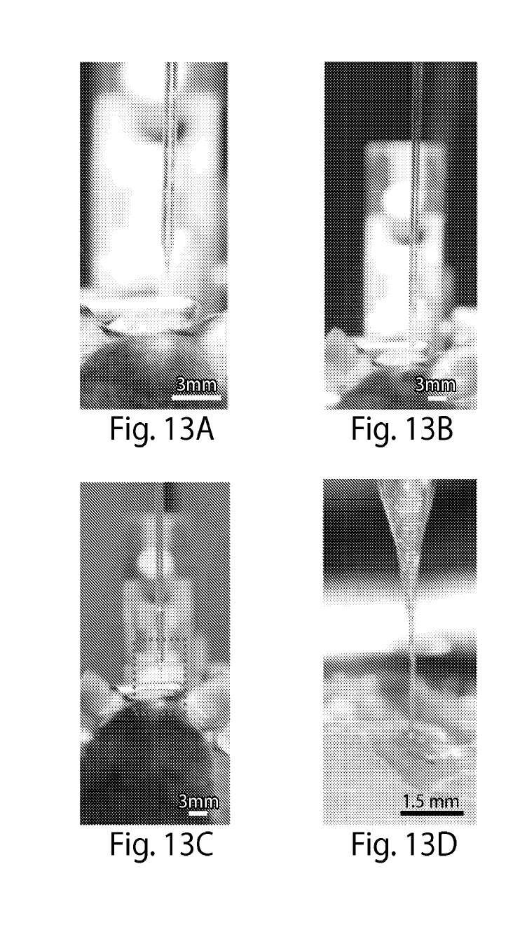

In this example, in vivo chronic implantation experiments were performed by stereotactically injecting electronics into rodent brain tissue with a 0.5 mm diameter drilled hole from craniotomy. The injection follows steps illustrated in FIG. 10D and 13A. Specifically, 2-mm-wide electronics were injected into the tissue-dense hippocampus region of the mice (FIG. 5E) through a 100-micrometer inner diameter glass needle controlled by microinjector, which allowed trace amount of solution (<1 microliters) to be injected with electronics in each injection. Fluorescence imaging of coronally sliced brain tissue showed that the electronics unfolded after 5 weeks and settled into the hippocampus region with little interruption to the layered structure of neurons (FIG. 5E). Notably, the neurons had grown together with the ribbons of electronics (FIG. 5F). The horizontal slice with immunostaining for astrocytes and microglial further showed a reduced chronic tissue response with little gliosis and immunoreactivity at the injection site, and also the area to which the mesh finally settles. Specifically, microglia adjacent to the electronics ribbon were not seen by Iba-1 staining (FIG. 5G).

FIG. 10 shows control experiments of electronics inside a needle. FIG. 10A shows an optical image showing a 5-mm-wide mesh injected through a 400-micrometer glass needle. Arrow indicates the direction of injection. FIG. 10B is a top view of a 3D-reconstructed confocal images correspond to FIG. 10A. FIG. 10C shows images at cross-section as indicated by white dashed line in FIG. 10B. The white dashed circle in FIG. 10C highlights the distribution of mesh in the needle cross-section (highlighted by white dashed line in FIG. 10B). FIG. 10D are schematics of a thin film electronics for injection showing supporting and passivation polymer and metal lines. FIGS. 10E-10F are images showing SU-8 film/metal with width below lmm can be injected through a 400-micrometer needle.

FIG. 13 shows delivery of injectable electronics in an example in vivo system, with the process of stereotactic injection of injectable electronics. The electronics are loaded into a glass needle in FIG. 13A. After injection of device region into tissue (FIG. 13B), the needle is withdrawn to extrude and the I/O region is injected on the outside of skull (FIG. 13C). FIG. 13D shows a zoomed-in region of dashed box highlighted in FIG. 13C.

To further demonstrate the potential of the geometrical restoration of the injectable electronics in cavity as well as its uniqueness for potential applications in cell therapy, certain electronics were injected into the cavity of the lateral ventricle to target the subventricular zone region because the cells in this region have the well-known of capability of regeneration and long-distance migration, and the related proposed neuronal replacement therapy. The same electronics as above were also stereotaxically injected into the lateral ventricle region through a 100-micrometer glass needle. Since the electronics behaved like a synthetic polymeric network, a relatively large amount of electronics could continuously be injected into the lateral ventricle to ensure that the electronics, and when unfolded, coudld be in contact with the lateral ventricle wall.

After 5 weeks, immunostaining of horizontal slice showed that electronics unfolded into a volume with 1.5-mm diameter covering the inner area of lateral ventricle and connecting the lateral walls. Immunostaining showed that the ribbons from electronics in contact with the striatum and stria terminalis interpenetrated with the cells merging into the astrocytic-characteristic tube-like structure. Control experiments from the same rodent also showed the same level of glial fibrillary acidic protein (GFAP) expression demonstrating little chronic tissue response to the electronics. Importantly, there was migration of neural outgrowth cells from both sides of the lateral ventricle into the interior space of the unfolded mesh in the cavity. Those cells formed high density and tight junctions on the ribbons of electronics in chain-structures, which followed the direction of ribbons from electronics.

FIG. 5 shows syringe injectable electronics for biological system. FIG. 5A is a schematic shows injecting electronics into matrigel together with cells, including the mesh structure of injectable electronics and cells. FIG. 5B are images showing that the device unfolds after being injected into matrigel for 72 hours. FIG. 5C are confocal fluorescence images of a 100-micrometer projection, showing the interpenetration between neurons and ribbons of injectable electronics after co-injected into Matrigel for 14 days. The image shows both the mesh and beta-tubulin staining for neurons. FIG. 4D, I is a schematic showing stereotactic injection of injectable electronics into an in vivo system. II is an optical image showing the stereotactic injection of injectable electronics into mice brain. The schematic shows that when injectable electronics were injected into the brain, into the hippocampus (III) as well as the lateral ventricle cavity in the brain, they unfolded. (IV) shows the mesh structure of the injectable electronics, and I/O pads for electrical connections. Dashed lines indicate direction of horizontal slicing for imaging.

FIG. 4E shows bright-field and epi-fluorescence image of the coronal slice in FIG. 4D, III, showing that the electronics were injected into the hippocampus. DAPI staining used. FIG. 4F shows a projection of 30-micrometer-thick volume from the zoomed-in region by white dashed box in FIG. 4E, with neurons interfacing with the electronics. FIG. 4G is a confocal image of a 30-micrometer horizontal slice shows staining for astrocytes, active microglia, and nuclei respect to the device at the position indicated by the dashed line in FIG. 4D, III. FIG. 4H is a projection of a 100-micrometer-thick volume for a device injected into cavity inside brain (lateral ventricle) at the position indicated by blue dashed line in FIG. 4D, IV. FIG. 41 is a projection of a 30-micrometer-thick volume for the zoomed-in region highlighted by white dashed box in FIG. 4H, showing the interface between the electronics and subventricular zone. FIG. 4J is a 3D reconstruction of the region highlighted by white box in FIG. 4H, including DAPI staining, the SU-8 ribbon, and reflections from the metal within mesh.

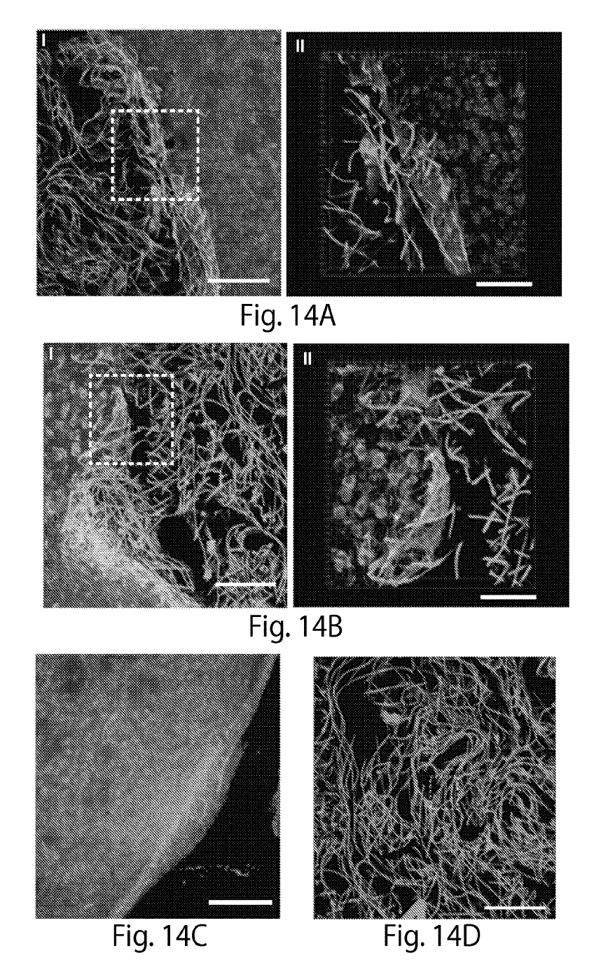

FIG. 14 shows the interface between electronics and tissue in an example in vivo system. FIG. 14A is a projection of 30-micrometer-thick volume slices, showing the interface between electronics in in vivo with a subventricular zone (I), and 3D reconstruction of the dashed box highlighted zoomed-in region (II). FIG. 14B is a projection of a 30-micrometer-thick volume of slice shows the interface between electronics in in vivo with staria (I) and a 3D reconstruction of the dashed box highlighted in the zoomed-in region (II). DAPI, SU-8 and NeuN, and GFAP are indicated. FIG. 14C is a control sample shows subventricular zone without a device. FIG. 14D is a projection of an 80-micrometer-thick volume for the region highlighted by the white box in FIG. 5H. Scale bar is 160 micrometers.

These results, together with the capability of electronics to monitor cellular electrophysiological and pharmacological activity, show potential applications. For example, some embodiments bay be directed to using use injectable electronics to directly mobilize and monitor the adult stem neurons from lateral ventricle region to brain injury for therapy.

### EXAMPLE 7

This example provides various materials and methods used in the above examples.

Freestanding injectable electronics were fabricated on nickel relief layer. See, e.g., International Patent Application No. PCT/US14/32743, filed Apr. 2, 2014, entitled “Three-Dimensional Networks Comprising Nanoelectronics,” by Lieber, et al., incorporated herein by reference. The electronics were modified by poly-D-lysine (MW 70,000 to 150,000, Sigma-Aldrich Corp.) and then loaded into syringe with metal gauge needle by glass pipette or loaded into glass needle pulled by a commercial available pipette puller (Model P-97, Sutter Instrument). A microinjector (NPIPDES, ALA Scientific instruments Inc.) or manually controlled syringes (Pressure Control Glass Syringes, Cadence, Inc.) were used to inject electronics. The electronics structure in glass channels and immunostaining of cells and tissue were characterized by Olympus Fluoview FV1000 system. ACF (AC-4351Y, Hitachi Chemical Co.) bonding was conducted by home-made or commercial bonding systems (Fineplacer Lambda Manual Sub-Micron Flip-Chip Bonder, Finetech, Inc.) with a flexible cable (FFC/FPC Jumper Cables PREMO-FLEX, Molex). Recording was amplified with a multi-channel preamplifier, filtered with a 3 kHz low pass filter (CyberAmp 380), and digitized at a 50 kHz sampling rate (Axon Digi1440A).

Nanowire Synthesis. Single-crystalline nanowires were synthesized using the Au nanocluster-catalyzed vapor-liquid-solid growth mechanism in a home-built chemical vapor deposition (CVD) system. Au nanoclusters (Ted Pella Inc. Redding, Calif.) with 30 nm diameters were dispersed on the oxide surface of silicon/SiO₂ substrates (600 nm oxides, n-type 0.005 V·cm, Nova Electronic Materials, Flower Mound, Tex.) and placed in the central region of a quartz tube CVD reactor system. Uniform 30-nm p-type silicon nanowires were synthesized. In a typical synthesis, the total pressure was 40 torr, and the flow rates of SiH₄, diborane (B₂H₆, 100 ppm in H₂), and hydrogen (H₂, semiconductor grade) were 2, 2.5, and 60 standard cubic centimeters per minute (SCCM), respectively. The silicon-boron feed-in ratio was 4,000:1, and the total nanowire growth time was 30-60 min.

Freestanding syringe injectable electronics fabrication. Key steps used in the fabrication of the syringe injectable electronics included the following: (1) Thermal deposition were used to deposit a 100-nm nickel metal layer over the whole silicon wafer (600-nm SiO₂ or 100-nm SiO₂/200-nm Si₃N₄, n-type 0.005V·cm, Nova Electronic Materials, Flower Mound, Tex.), where the nickel served as the final relief layer for freestanding electronics. (2) A 300- to 400-nm layer of SU-8 photoresist (2000.5; MicroChem Corp., Newton, Mass.) was spin cast (3000 rpm) over the entire chip followed by prebaking at 65° C. and 95° C. for 2 and 4 min, respectively. (3) Photolithography was used to pattern the bottom SU-8 layer for passivating and supporting the whole device structure. After postbaking (65° C. and 95° C. for 2 and 4 min, respectively), SU-8 developer (MicroChem Corp., Newton, Mass.) was used to develop the SU-8 pattern. Those areas exposed to UV light became indissoluble to SU-8 developer, and other areas were dissolved by SU-8 developer. The SU-8 patterns were cured at 180° C. for 20 min. (4) A 300- to 400-nm layer of SU-8 photoresist was spin cast (3000 rpm) over the entire chip, followed by prebaking at 65° C. and 95° C. for 2 and 4 min, respectively, then (5) the synthesized nanowires were directly printed from growth wafer over the SU-8 layer by the contact printing. Photolithography was used to define regular patterns on the SU-8. After postbaking (65° C. and 95° C. for 2 and 4 min, respectively), SU-8 developer (MicroChem Corp., Newton, Mass.) was used to develop the SU-8 pattern. Those nanowires on the non-exposed area were removed by further washing away in isopropanol solution 30 s for twice leaving those selected nanowires patterned on the regular patterns of SU-8 structure. The SU-8 patterns were cured at 180° C. for 20 min. (6) To fabricate metal electrode electrophysiological sensor, photolithography and electron beam deposition were used to define and deposit 20×20 micrometer² Pt pad. (7) Photolithography and thermal deposition were used to define and deposit the metal contact to address each nanowire device and form interconnections to the input/output pads for the array. For the general metal contact region, in which the metal is nonstressed, symmetrical Cr/Au/Cr (1.5/50-80/1.5 nm) metal was sequentially deposited followed by metal liftoff in acetone. For device regions in which the metal is nonstressed, symmetrical Cr/Pd/Cr (1.5/50-80/1.5 nm) metal was sequentially deposited followed by metal liftoff in acetone. For device regions in which metal is stressed for organizing into 3D structure, nonsymmetrical Cr/Pd/Cr (1.5/50-80/50-80 nm) metal was sequentially deposited followed by metal liftoff in acetone. (8) A 300- to 400-nm layer of SU-8 photoresist was spin cast (3000 rpm) over the entire chip, followed by prebaking at 65° C. and 95° C. for 2 and 4 min, respectively. Then lithography was used to pattern the top SU-8 layer for passivating the whole device structure. The structure was post-baked, developed, and cured by the same procedure as described above. (9) A 300- and 500-nm thick layers of LOR 3A and S1805 (MicroChem Corp., Newton, Mass.) photoresist can be deposited by spin-coating and defined by photolithography to further protect the device region if necessary. (10) The 2D syringe injectable electronics were released from the substrate by etching of the nickel layer (Nickel Etchant TFB, Transene Company Inc.) for 3 to 4 hours at 25° C. (11) If the device region was protected by photoresist protection layer, the electronics were transferred into deionized (DI) water for rinsing and then dried on substrate, exposed in ultraviolet light (430 nm, 120 s) to sensitize the photoresist protection with subsequently immersed in developer solution (MF-CD-26, MicroChem Corp., Newton, Mass.) to dissolve the protection on device region.

Structure characterization. Scanning electron microscopy (SEM, Zeiss Ultra55/Supra55VP field-emission SEMs) was used to characterize the structure of electronics. Bright-field and dark-field optical micrographs of samples were acquired on an Olympus FV1000 system using FSX-BSW software (ver. 02.02). Fluorescence images were obtained by doping the SU-8 resist solution with Rhodamine 6G (Sigma-Aldrich Corp., St. Louis, Mo.) at a concentration less than 1 micrograms/mL before deposition and patterning by Olympus FSX100 confocal microscopy system. ImageJ (ver. 1.45i, Wayne Rasband, National Institutes of Health, USA) was used for 3D reconstruction and analysis of the confocal and epi-fluorescence images.

Surface modification of the electronics. The freestanding electronics was transferred into DI water by glass pipette to remove nickel etchant or developer solution. Then the electronics was transferred and soaked into poly-D-lysine (PDL, 0.5-1.0 mg/ml, MW 70,000 to 150,000, Sigma-Aldrich Corp., St. Louis, Mo.) aqueous solution for 2 to 12 hours at 25° C. for surface modification. After surface modification, the electronics was transferred into PBS (HyClone™ Phosphate Buffered Saline, Thermo Fisher Scientific Inc., Pittsburgh, Pa.) buffer solution for future use.

Mesh structure design. A. General tilted mesh electronics: The structure is illustrated in FIG. 7. The ribbon along the injection direction is called the longitudinal ribbon and the ribbon perpendicular to the injection direction is called the transverse ribbon. The transverse ribbons are tilted 45° counterclockwise to transverse direction on the mesh plane forming 45° angle to longitudinal ribbons. Metal contacts were mainly encapsulated in longitudinal ribbons. Some transverse ribbons also contained metal contacts to form the source-drain of field-effect transistor. For passive metal electrode electronics, only longitudinal ribbons contained metal contact. Silicon nanowire devices and passive metal electrodes were patterned either on the longitudinal ribbons in the center of unit cells or patterned separately in a beam in the longitudinal direction on the transverse ribbons in the center of unit cells to reduce strains for device during injection. For the ribbons containing metal contact lines, the 100-nm thick metal lines were encapsulated in the middle of two 350-nm thick SU-8 layers. For the ribbons without metal contact lines, the total SU-8 thickness was about 700 nm. Transverse ribbons and longitudinal ribbons together formed mesh with periodic unit cells.

The dimensions of all unit cells are identical across the whole mesh in these experiments. Design #1 was used for injection by needle with inner diameter larger than 200 micrometers (FIG. 7A). The width of the mesh was 5 to 15 mm. The length of unit cell was 333 micrometers and the width of unit cell was 250 micrometers. All the SU-8 layers in these experiments were 20 micrometers in width and all of the metal layers were 10 micrometers in width. Design #2 was used for injection by needle with inner diameter smaller than 200 micrometers (FIG. 7B). The width of mesh was 2 to 5 mm. The length of unit cell was 333 micrometers and the width of unit cell was 250 micrometers. SU-8 layers in longitudinal ribbons were 20 micrometers in width and the SU-8 layers in transverse ribbons were 5 to 10 micrometers in width. Metal layers in longitudinal ribbons were 10 micrometers in width and metal layers in transverse ribbons are 2 to 5 micrometers in width.

Control orthogonal mesh electronics sample. The transverse ribbons were perpendicular to the longitudinal ribbons to form an orthogonal mesh with the same periodic unit cell structure. All metal line patterns, thickness and width of ribbons are the same as design #1 of tilted trasverse ribbons electronics. The width of electronics was 5 to 15 mm for testing.

Control thin film electronics sample. The thickness of SU-8 was 700 nm. The metal line patterns were the same as design #1 of tilted mesh electronics. The width of electronics was 0.1 to 5 mm.

Glass needle and fluidic channel preparation. The glass needles were by using a conventional pipette puller (Model P-97, Sutter Instrument, CA) and glass tube (30-0057, Harvard Apparatus) following the parameters: Heat: Ramp +25, Pull: 0, Velocity: 140, Time: 100 and Pressure: 200. For a clean-cut needle with inner diameter from 20 to 200 micrometers, ceramic tiles (#CTS, Sutter Instrument, CA) were used to score the glass tip checked by optical microscope with subsequent mechanical break.

For the channels used for imaging, the pulling was halted and suspended in the middle to not completely break the glass tube (VWR International, LLC, Radnor, Pa.). The channel size was characterized by confocal fluorescence imaging. Rodamine-6G (Sigma-Aldrich Corp., St. Louis, Mo.) solution was filled into the channel for imaging. For a channel inner diameter smaller than 300 micrometers, epoxy glue was used to increase stability of channel preventing channel broken during imaging.

Surface-to-volume-ratio calculation. The surface-to-volume-ratio of a ribbon or a film (length, l; width, w; height, h) was calculated as:

2(lw+lh+wh)/lwh=2(1h+1/w+1/l).

For a typical thin film of 10 micrometers height, with much larger length and width, the surface-to-volume-ratio is ˜2/h=0.2 micrometers ⁻¹. For a typical ribbon (large length l) in the mesh structure with 5 micrometers and 0.7 micrometers in width and height respectively, the surface-to-volume-ratio was ˜2/h+2/w=3.25 micrometers⁻¹.

Injection by metal gauge needles. After surface modification, the electronics were transferred into a syringe (Pressure Control Glass Syringes, Cadence, Inc., Cranston, R.I.) with a metal gauge needle (Veterinary Needles, Cadence, Inc., Cranston, R.I.) by a glass pipette (Disposable Pasteur Pipets, Lime Glass, VWR International, LLC, Radnor, Pa.). The orientation and unfolded structure of the electronics in the syringe should be performed to prevent any buckles. Press the syringe and allow the tip part of the electronics be loaded into the needle.

Injection by glass needle. After surface modification, the electronics were transferred into a syringe with a metal gauge needle by a glass pipette. The orientation and unfolded structure of the electronics in the syringe should be performed to prevent any buckles. The syringe was connected to glass needle by plastic tubing. Press the syringe and allow the tip part of the electronics be loaded into the needle. To better control injection process, the microinjector (NPIPDES, ALA Scientific instruments Inc., Farmingdale, N.Y.) and patch-clamp set-up (Axonpatch 200B, Molecular Devices, LLC, Sunnyvale, Calif.) were used for control the injection process. The electronics were directly loaded into the glass needle illustrated by FIG. 8A as follows: (1) A plastic tube was connected to the tip end of glass needle and connected to a syringe. (2) The electronics was drawn in into the rear part of glass needle. (3) The plastic tube was removed from glass needle and the needle was mounted onto patch-clamp set-up and connected to microinjector or syringe for injection (FIG. 8B).

FIG. 8 shows the controllable injection process. FIG. 8A is a schematic showing how the mesh electronics was stepwise loaded into glass needle. In (I), the tip of the glass needle was connected to a syringe by a plastic tube. The injectable electronics device was drawn into the needle from the end of glass needle. (II) shows the electronics device loaded into the glass needle. (III) shows the glass needle was mounted to a patch-clamp setup for injection. FIG. 8B shows a setup of controllable injection system. Dashed arrow highlights the plastic tube connecting the syringe with glass needle through the patch-clamp setup. FIG. 8C shows optical images of a typical electronics transfer during injection process. (I-VI) shows that the electronics was gradually injected into free solution by a micro-injector with 1 bar pressure, 10 ms pulse (before dashed line in FIG. 8D) and 50 ms pulse (after the dashed line in FIG. 8D) injection times for each step. FIG. 8D shows the injected length of electronics vs. number of injection.

Yield of injection. To obtain the yield of electronics after injection, the conductance of nanowire devices before and after injection through needles was compared as following procedure: (1) As-made 2D electronics were partially immersed in etchant solution (Nickel Etchant TFB, Transene Company Inc., Danvers, Mass.) for 3 to 4 hours at 25° C. to firstly release nickel layer under the I/O region of the electronics. Then, the electronics was transferred to DI water and dried in ethanol, while the released I/O region was unfolded on the substrate. (2) After the electronics dried completely, the left nickel layer was etched in etchant solution for 1 to 2 hours at 25° C., after which the electronics would be transferred to DI water and dried in ethanol to allow active device region to be unfolded on the substrate. Because the I/O pads covered larger region than electronics, these two-step etching process reduced the etching time for active device region. (3) After completely drying, the electronics adhered weakly on the wafer, and could be easily removed from the substrate afterwards. Conductance (G₀) for each device was measured by a probe station (Desert Cryogenics, Model 4156C) which was back plane grounded. Current-voltage (I-V) data were recorded using an Agilent semiconductor parameter analyzer (Model 4156C) with contacts to device through probe station. Devices with conductance above 100 nS were accounted as initial devices with total number N₀ in this stage. (4) After conductance measurement, the electronics on substrate were immersed in DI water for 4 to 6 hours until it released from the substrate and fully suspended in the solution. (5) The electronics were transferred through glass pipette to PDL aqueous solution for surface modification as described above. (6) The electronics were loaded by glass pipette into syringe with gauge metal needle and injected through needle with different inner diameters (from 100 to 600 micrometers) into a chamber with the I/O part unfolded near the chamber on a substrate (FIG. 1F). (7) Ethanol was used to rinse and dry the I/O part. (8) Conductance (G₁) for each device was measured again with the same probe station under same condition, and the total number of survived devices with G₁ above 100 nS was N₁. Yield and conductance changes in FIG. 1G were calculated as (N₁/N₀) and (G₁-G₀)/G₀, respectively.

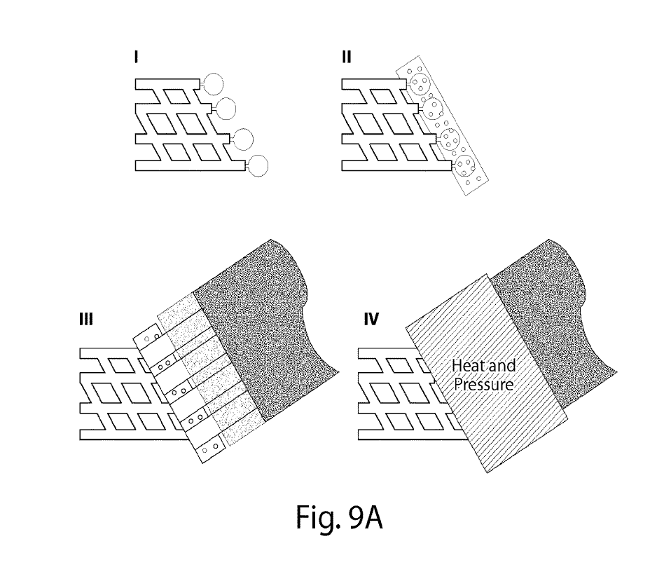

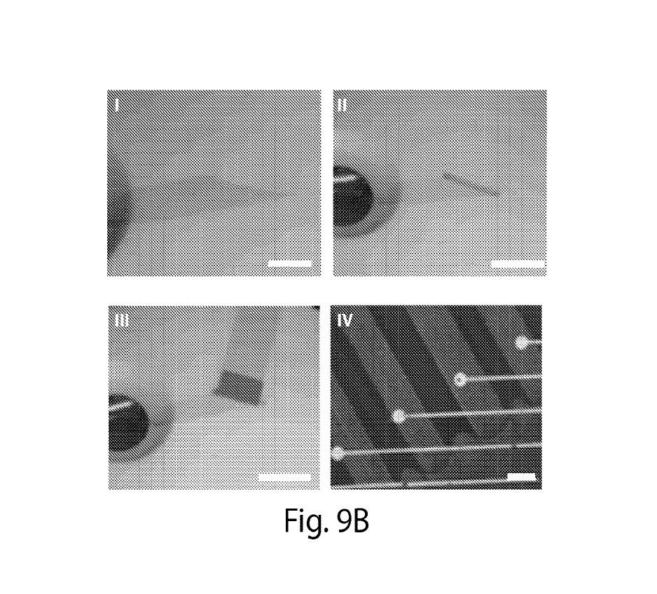

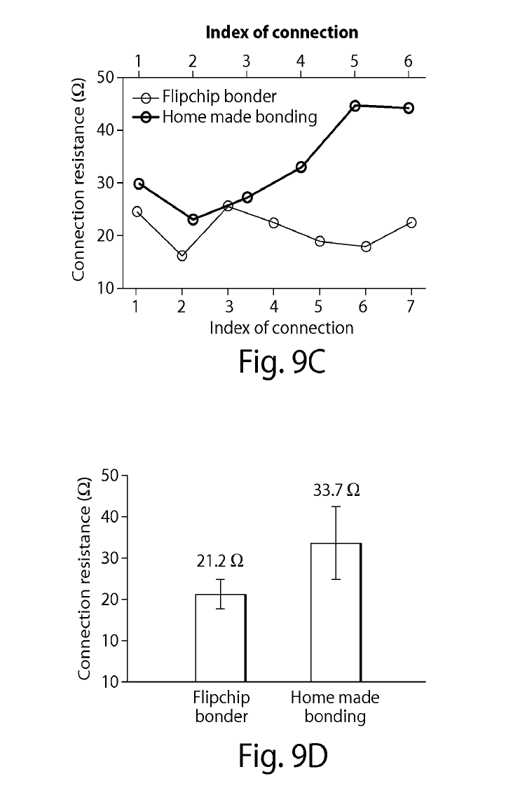

ACF bonding process. After fabrication, the electronics were injected through a syringe into solution, soft matters, biomaterials or tissues, with I/O part injected outside the target materials. DI water and other solvents (PBS, culture medium, hexane, etc.) were introduced to facilitate unfolding the I/O region, after which the I/O region was rinsed and dried with ethanol (ethanol, 190 proof (95%), VWR International, LLC, Radnor, Pa.) (FIGS. 9A and 9B). For the connection to measurement setup, the unfolded and dried I/O region of injectable electronics was bonded to the flexible cable (FFC/FPC Jumper Cables PREMO-FLEX, Molex, Lisle, Ill.) through an anisotropic conductive film (ACF, AC-4351Y, Hitachi Chemical Co. America, Ltd., Westborough, Mass.). The ACF was 1.2 mm wide with conductive particles ˜3 micrometers in diameter.

Firstly, an ACF with protective layer was positioned on the I/O region, and presealed after being heated to 90° and a pressure of 1 MPa for 1 min with a homemade hot bar or commercial bonding system (Fineplacer Lambda Manual Sub-Micron Flip-Chip Bonder, Finetech, Inc., Manchester, N.H.) to tack it on the I/O part with protective layer removed. Then the flexible cable was placed on the ACF and aligned. At last, the endsealing was made with a temperature of 190 to 210° C. in ACF and a pressure of 4 MPa on the top for 5 min applied by homemade hot bar or a commercial bonding system. In order to demonstrate the adhesion strength of the interface between I/O pads and flexible cable, the structure was peeled from the substrate and examined by optical microscopy (FIG. 9B, IV).

The connection resistance of ACF was measured to investigate the influence of bonding on electrical properties of devices (FIG. 9C-9D). The conductance of each device was measured by the probe station as R₀ and R₁ before and after ACF bonding, respectively. The connection resistance for each I/O pad (100 micrometers diameter) was calculated as (R₁-R₀)/2, illustrated in FIG. 9C. The calculated connection resistance after ACF bonding with commercial bonder and homemade bonding is ca. 21.2 ohms and ca. 33.7 ohms respectively (FIG. 9D), below 0.05% of typical nanowire resistance. The insulation resistance between I/O pads without circuits was over 10¹⁰ ohms. These measurements and calculation results demonstrated that ACF bonding had little influence on electrical properties of injectable electronics, which ensured reliable measurement of injectable electronics in many kinds of applications afterwards.

FIG. 9 shows the bonding process used here. FIG. 9A is a schematic showing the steps of bonding process. (I) shows that the I/O region of the electronics device was unfolded on the substrate, (II) shows the ACF film was attached to I/O region, (III) shows a flexible cable aligned with the I/O pads and (IV) shows that a hot bar was applied to the bonding region to make the connection. FIG. 9B, (I-III) shows optical images correspond to the steps in FIG. 9A. Scale bar: 0.5 cm (I), 1 cm (II, III). (IV) shows the I/O pads of electronics were bonded with flexible cable after heating and pressure applied by the hot bar. Scale bar: 200 micrometers. FIG. 9C shows the connection resistance of the ACF film bonded by flipchip bonder (upper trace) and a homemade bonding system (lower trace). FIG. 9D shows the statistic results of connection resistance data in FIG. 9C, showing the average value and standard deviation.

Imaging of electronics in glass channel. Electronics with different widths and mesh structures were injected into the glass channels following the same injection process described above. However, the electronics were only partially injected through the needle. Confocal fluorescence microscope was used to image the 3D structure of the electronics in the glass needle. ImageJ was used to re-slice the 3D reconstructed images of device in the longitudinal direction by the step of 1 micrometers.

Mechanical simulation, bending stiffness simulation. The bending stiffness of the devices was estimated with different structures by finite element software ABAQUS. A unit cell is used for the simulation, and the tilt angle a (alpha) is defined in FIG. 3A. The devices were modeled with shell elements. The longitudinal ribbons were partitioned into a one-layer part and a three-layer part (FIG. 7C). A homogeneous section with 700-micrometer thick SU-8 is assigned to the transverse ribbons, while a composite section with three layers of 300-nm thick SUB, 100-nm thick gold and another 300-nm thick SU-8 was assigned to the three-layer part of the longitudinal ribbons. Both SU-8 and gold were modeled as linear elastic material, with Young's moduluses of 2 GPa and 79 GPa, respectively. To calculate the longitudinal and transverse bending stiffness, a fixed boundary condition was set at one of the ends parallel with the bending direction, and a small vertical displacement d is added at the other end. The external work w to bend the device is calculated. The effective bending stiffness of the device was defined as the stiffness required of a homogenous beam to achieve the same external work w under the displacement d. Therefore, the effective bending stiffness per width of the device can be estimated as:

$$D = \frac{2 {Wl}^{3}}{3 {d}^{2} b} ,$$

with b the width of the unit cell parallel with the bending direction, and l the length of the unit cell perpendicular to the bending direction. The bending stiffness for unit cell bent in the transverse direction decreases with the tilt angle a (alpha), while the bending stiffness for a unit cell bent in the longitudinal direction increases with a (alpha) (FIG. 3B).

Injection simulation. A unit cell with the tilted angle α (alpha)=45° was further simulated going through a needle. The unit cell was bent by a rigid shell with radius of curvature R (FIG. 3C). A fixed boundary condition was set on one of the end of the device parallel with the bending direction. The distribution of the maximal principal strain εₘ is shown in the inset of FIG. 3C. When the radius of the needle R is 300 micrometers, the highest maximal principal strain is as small as 0.167%; when the radius of the needle R is 100 micrometers, εₘ reached around 0.513%. The dependence of the highest maximal principal strain εₘ of the unit cell on the curvature 1/R is linear as shown in FIG. 3C, with different sizes of the mesh structures. The two corresponding fitting relations were εₘ=0.499/R and εₘ=0.473/R, respectively.

Dimensional analysis of integration of the device with cells. When the electronics were injected into tissues, the flexibility of the device and the survival of cells, especially in long-term chronic implantation, was studied. When the device is too rigid to bend, chronic damage could be induced by mechanical mismatch. Here, a dimensionless number D/γtL is defined, where D is the bending stiffness per width of the electronics as calculated in FIG. 3B, γ (gamma) is the membrane tension of cells, t is the thickness of the electronics and L is the length of the electronics. Since the bending curvature of the device scales as ≈1/L, the bending energy scales as ≈Dw/L, with w the width of the device. The surface membrane energy due to the insertion of the electronics scaled as 18 γwt. Therefore, the ratio of the bending energy and the surface energy gives the dimensionless number D/γtL, which describes the flexibility of the device compared to the membrane tension of cells. The electronics used here have the properties of D˜0.01 nN m, t˜1 micrometer, and L˜1 cm, and typical cells have γ˜1 mN/m·D/γtL˜1. Therefore, the electronics used in this example had the proper flexibility for it to function well and integrate with cells.

Preparation of electronics with extreme twisting structure. The freestanding electronics was suspended into DI water after modification. With the glass pipette transferring, the electronics was folded onto a glass substrate with DI water. The hybrid structure was dried using a critical point dryer (Autosamdri 815 Series A, Tousimis, Rockville, Md.) and stored in the dry state prior to be characterized by SEM (FIG. 11C).

Inject electronics in soft matters. PDMS pre-polymer components were prepared in a 10:1 (base:cure agent; Sylgard 184, Dow Corning Corporation, Midland, Mich.) weight ratio at first, and diluted by hexane (n-hexane 95% optima, Fisher Scientific Inc., Pittsburgh, Pa.) in a 1:3 (PDMS:hexane) volume ratio. The cavity for injection was formed by two pieces of cured PDMS (cured at 65° C. for 2 hours; Sylgard 184, Dow Corning Corporation, Midland, Mich.). The electronics were transferred from water to ethanol after etching, dissolved in PDMS/hexane solution and then loaded into glass syringe with 18 gauge metal needle. The device region was injected into the cavity (FIG. 12A, I), with the I/O region injected outside the cavity on a silicon wafer or a glass side (VistaVision Microscope Slides, Plain and Frosted, VWR International, LLC, Radnor, Pa.). Hexane was used to wash away PDMS residues on the I/O region, after which the I/O region were unfolded with alcohol (FIG. 12A, II). The transition part of electronics from PDMS to substrate was fixed by Kwik-Sil (World Precision Instruments, Inc., Sarasota, Fla.) silicone elastomer to avoid damage to the device during the drying process. Finally, the hybrid structure of PDMS and electronics was cured at room temperature for 48 hours.

The I/O pads were bonded to flexible cable through ACF as the process described above. The piezoelectric response to strain of the nanowire devices was calibrated using homemade clamp device with linear translocation stages under tensile or compressive strain in x direction (FIGS. 4B, 12C, and 12D), where the strain was calculated from the relative length change (ΔL/L=0.5 mm/54 mm=0.9%). The strain field caused by point load in z direction was determined in experiments where the hybrid structure with calibrated nanowire strain sensors was subject to non-uniform deformations.

Inject electronics in Matrigel with and without neurons. Poly-D-lysine modified electronics was transferred into PBS solution and then into Neurobasal™ medium (Invitrogen, Grand Island, N.Y.). The electronics were loaded into metal syringe needle as described above. Matrigel™ (BD Bioscience, Bedford, Mass.) was diluted into 30% (v/v) with neuron culture medium and polymerized at 37° C. The electronics was injected into polymerized Matrigel. The hybrid structure was incubated in 37° C. to investigate the unfolding of electronics in Matrigel™.

Hippocampal neurons (Gelantis, San Diego, Calif.) were prepared using a standard protocol. In brief, 5 mg of NeuroPapain Enzyme (Gelantis, San Diego, Calif.) was added to 1.5 ml of NeuroPrep Medium (Gelantis, San Diego, Calif.). The solution was kept at 37 ° C. for 15 min, and sterilized with a 0.2 micrometer syringe filter (Pall Corporation, MI). Day 18 embryonic Sprague/Dawley rat hippocampal tissue with shipping medium (E18 Primary Rat Hippocampal Cells, Gelantis, San Diego, Calif.) was spun down at 200 g for 1 min. The shipping medium was exchanged for NeuroPapain Enzyme medium. A tube containing tissue and the digestion medium was kept at 30° C. for 30 min and manually swirled every 2 min, the cells were spun down at 200 g for 1 min, the NeuroPapain medium was removed, and 1 ml of shipping medium was added. After trituration, cells were isolated by centrifugation at 200 g for 1 min, then resuspended in 5-10 mg/ml Matrigel™ at 4° C. Matrigel with neurons were mixed with electronics at 4° C. and then loaded into syringe with a metal gauge needle. The electronics and neurons were co-injected into 30% (v/v) polymerized Matrigel™ in a culture plate and then placed in an incubator to allow the Matrigel™ to gel at 37° C. for 20 min. Then, 1.5 ml of NeuroPure plating medium was added. After 1 day, the plating medium was changed to Neurobasal™ medium (Invitrogen, Grand Island, N.Y.) supplemented with B27 (B27 Serum-Free Supplement, Invitrogen, Grand Island, N.Y.), Glutamax (Invitrogen, Grand Island, N.Y.) and 0.1% Gentamicin reagent solution (Invitrogen, Grand Island, N.Y.). The in vitro co-cultures were maintained at 37° C. with 5% CO₂ for 14 days, with the medium changed every 4-6 days.

Immunostaining and imaging of neurons and electronics. The cells were fixed with 4% paraformaldehyde (Electron Microscope Sciences, Hatfield, Pa.) in PBS for 15-30 min, followed by 2-3 washes with ice-cold PBS. Cells were pre-blocked and permeabilized (0.2-0.25% Triton X-100 and 10% feral bovine serum (F2442, Sigma-Aldrich Corp. St. Louis, Mo.) for 1 hour at room temperature. Next, the cells were incubated with primary antibodies Anti-neuron specific beta-tubulin (in 1% FBS in 1% (v/v)) for 1 hour at room temperature or overnight at 4° C. Then, the cells were incubated with the secondary antibodies AlexaFluor-546 goat anti-mouse IgG (1:1000, Invitrogen, Grand Island, N.Y.). For counter-staining of cell nuclei, the cells were incubated with 0.1-1 microgram/mL Hoechst 34580 (Molecular Probes, Invitrogen, Grand Island, N.Y.) for 1 min.

Mouse Surgery. Adult (25-35 g) male C57BL/6J mice (Jackson lab) were group-housed, giving access to food pellets and water ad libitum and maintained on a 12 h:12 h light: dark cycle. All animals were held in a facility beside lab 1 week prior to surgery, post-surgery and throughout the duration of the behavioral assays to minimize stress from transportation and disruption from foot traffic. All procedures were approved by the Animal Care and Use Committee of Harvard University and conformed to US National Institutes of Health guidelines.

Stereotaxic surgery. After animals were acclimatized to the holding facility for more than 1 week, they were anesthetized with a mixture of 60 mg/kg of ketamine and 0.5 mg/kg medetomidine (Patterson Veterinary Supply Inc., Chicago, Ill.) administered intraperitoneal injection, with 0.03 mL update injections of ketamine to maintain anesthesia during surgery. A heating pad (at 37° C.) was placed underneath the body to provide warmth during surgery. Depth of anesthesia was monitored by pinching the animal's feet periodically. Animal was placed in a sterotaxic frame (Lab Standard Stereotaxic Instrument, Stoelting Co., Wood Dale, Ill.) and a 1-mm longitudinal incision was made, and skin was resected from the center axis of the skull, exposing a 2 mm by 2 mm portion of the skull. The dura was incised and resected from the surface of the skull. Next, a 0.5 mm diameter hole was drilled into the frontal and parietal skull plates using a dental drill (Micromotor with On/Off Pedal 110/220, Grobet USA, Carlstadt, N.J.).

Sterile saline was swabbed on the brain surface to keep it moist throughout the throughout the surgery. A sterotaxic arm was used to clamp the needle containing the injectable electronics. The electronics were loaded into the needle by first connecting the glass needle to a syringe via a plastic tube and drawn into the end of the glass needle. The glass needle was then detached from the syringe and then mounted to a patch-clamp setup for injection. The glass needle had a diameter of 100 to 200 micrometers. The needle was lowered into the exposed brain surface and the syringe or microinjector was used to inject the electronics into the brain. The needle was lowered approximately 1 mm into the skull (Interaural: 6.16 mm, Bregma: −3.84 mm), to test the effects of deep brain and superficial layer injections. After injection, the needle is drawn out of the brain tissue and the I/O region was injected on the surface of the skull.

After injection, the skin that was retracted from the center axis was replaced and the incision was sealed with C&B-METABOND (Cement System). Anti-inflammatory and anti-bacterial ointment was swabbed onto the skin after surgery. A 0.3 mL intraperitoneal injection of Buprenex (Patterson Veterinary Supply Inc. Chicago, Ill., diluted with 0.5 ml of PBS) for 0.1 mg/kg was administered to reduce post-operative pain. Animals were observed for four hours after surgery and hydrogel was provided for food, and heating pad was on at 37° C. for the remainder of post-operative care. All procedures complied with the United States Department of Agriculture guidelines for the care and use of laboratory animals and were approved by the Harvard University Office for Animal Welfare.

Incubation and behavioral analysis. The animals were cared for every day for 3 days after the surgery and every other day after first 3 days. The animals were administered with 0.3 mL of Buprenex (0.1 mg/kg, diluted with 0.5 mL PBS) every 12 hours for 3 days. The animals were also observed every other day for behavioral changes. Animals, which were surgically operated on, were housed individually in cage with food and water ad libitum. The room was maintained at constant temperature on a 12-12 h light-dark cycle.

Brain tissue preparation for chronic immunostaining. (1) Mice underwent transcardial perfusion (40 mL PBS) and were fixed with 4% formaldehyde (Sigma, 40 mL) four weeks after the surgery. (2) Mice were decapitated and brains were removed from the skull and set in 4% formaldehyde for 24 hours as post fixation and then PBS for 24 hours to remove extra formaldehyde. Electronics was kept inside the brain throughout fixing process. (3) The brains were blocked, separated into the two hemispheres and mounted on the stage of vibratome (Vibrating Blade Microtome Leica VT1000 S, Leica Microsystems Inc. Buffalo Grove, Ill.). 50-100 micrometer vibratome tissue slices (horizontal and coronal orientations) were prepared for samples with staining for microglia, astrocytes and nuclei. 30-50 micrometer vibratome tissue slices (horizontal and coronal orientations) were prepared for samples with staining for neurons. For samples with the electronics injected in the lateral ventricle, the brains were blocked and then fixed in 1% (w/v) agarose type I-B (Sigma-Aldrich Corp., St. Louis, Mo.) to fix the position of the electronics in the lateral ventricle cavity and then mounted on the stage of vibratome. 100 micrometer vibratome tissue horizontal slices were prepared. Coronal slices allowed for cuts in a direction along the long axis of the injected electronics and horizontal slices allowed for cuts in a direction perpendicular to the long axis of the injected device.

Chronic Immunohistochemistry: Microglia, Astrocytes and Nuclei. (1) Sections were then cleared with 5 mg/mL sodium borohydride in HEPES-buffered Hanks saline (HBHS, Invitrogen) for 30 minutes, with 3 following washes with HBHS in 5-10 minute intervals. Sodium azide (4%) was diluted 100× in HBHS in all steps using HBHS. (2) The slices were incubated with 0.5% (v/v) Triton X-100 in HBHS for 30 min at room temperature. (3) The slices were blocked with 5% (w/v) FBS and incubated overnight at room temperature. (4) The slices were washed four times in 30 min intervals with HBHS to clear any remaining serum in the tissue. The slices were then incubated overnight at room temperature with the GFAP primary antibody (targeting astrocytes, 1:1000, Invitrogen #13-0300, lot #686276A) and rabbit anti-Iba-1 primary antibody (targeting microglia, 1:800, Wako #019-19741, lot #STN0674) containing 0.2% triton and 3% serum. (5) After the incubation period, slices were again washed four times for 30 minutes with HBHS, the slices were incubated with secondary antibody (1:200; Alexa Flour 546 goat anti-rat, 1:200; Alexa Fluor 488 goat anti-rabbit secondary antibody, Invitrogen, Carlsbad, Calif.), Hoechst 33342 (nuclein stain 1:150, Invitrogen #h-1399, lot #46C3-4) 0.2% Triton and 3% serum overnight. (6) After the final washes (four for 30 min each HBHS), the slices were mounted on glass slides with coverslips using Prolong Gold (Invitrogen) mounting media. The slides remained covered (protected from light) at room temperature, allowing for 12 hours of clearance before imaging.

Chronic Immunohistochemistry: Neuron. The slices were cleared with 5 mg/mL sodium borohydride in HBHS for 30 minutes, with 3 following washes with HBHS in 5-10 minute intervals. Then, the slices were incubated with 0.5% (v/v) Triton X-100 in HBHS for 30 min at room temperature. Next, sections were blocked with 5% (w/v) serum and incubated overnight at room temperature. Next, slices were washed four times in 30-minute intervals with HBHS to clear any remaining serum in the tissue. The slices were then incubated with primary antibody (NeuN, 1:200, AbCam #ab77315) in 0.3% Triton-X100 and 3% serum in PBS overnight at room temperature. After 24 hours, the sections were washed four times for 30 minutes in PBS and then counterstained with Hoechst 33342 (1:5000, Invitrogen #H35770). Prolong gold coverslips were used again to protect from light and allowed for 12 hrs of clearance before imaging. When the antibody solutions were first prepared, they included 0.3 Triton X-100 and 5% normal goat serum.

Immunostaining for electronics in the cavity of lateral ventricle. The slices were cleared with 5 mg/mL sodium borohydride in HBHS for 30 minutes, with 3 following washes with HBHS in 5-10 minute intervals. Then, the slices were incubated with 0.5% (v/v) Triton X-100 in HBHS for 30 min at room temperature. Next, sections were blocked with 5% (w/v) serum and incubated overnight at room temperature. Next, the slices were washed four times in 30-minute intervals with HBHS to clear any remaining serum in the tissue. The slices were then incubated with primary antibody (NeuN, 1:200, AbCam #ab77315) in 0.3% Triton-X100 and 3% serum in PBS overnight at room temperature. After 24 hours, the sections were washed four times for 30 minutes in PBS and then counterstained with Hoechst 33342 (1:5000, Invitrogen #H35770). Prolong gold coverslips were used again to protect from light and allowed for 12 hrs of clearance before imaging. When the antibody solutions were first prepared, they included 0.3 Triton X-100 and 5% serum.

### EXAMPLE 8

The following examples demonstrates syringe injection and subsequent unfolding of rationally-designed sub-micrometer-thick, centimeter-scale macroporous mesh electronics through needles with inner diameter as small as 100 micrometers. These results show that electronics can be injected into man-made and biological cavities, as well as dense gels and tissue with >90% device yield. Several applications of syringe injectable electronics as a general approach for interpenetrating flexible electronics with 3D structures are demonstrated, including (i) monitoring of internal mechanical strains in polymer cavities, (ii) tight integration and low chronic immunoreactivity with several distinct regions of the brain, and (iii) in vivo multiplexed neural recording. Moreover, syringe injection allows delivery of flexible electronics through a rigid shell, delivery of large volume flexible electronics that can fill internal cavities and co-injection of electronics with other materials into host structures, capabilities that are distinct from and open up unique applications for flexible electronics.

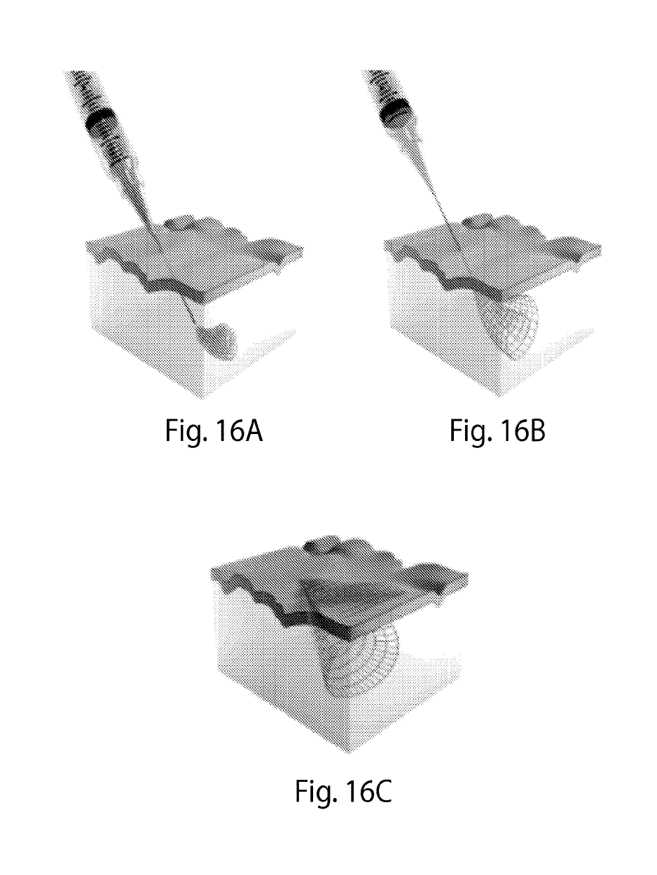

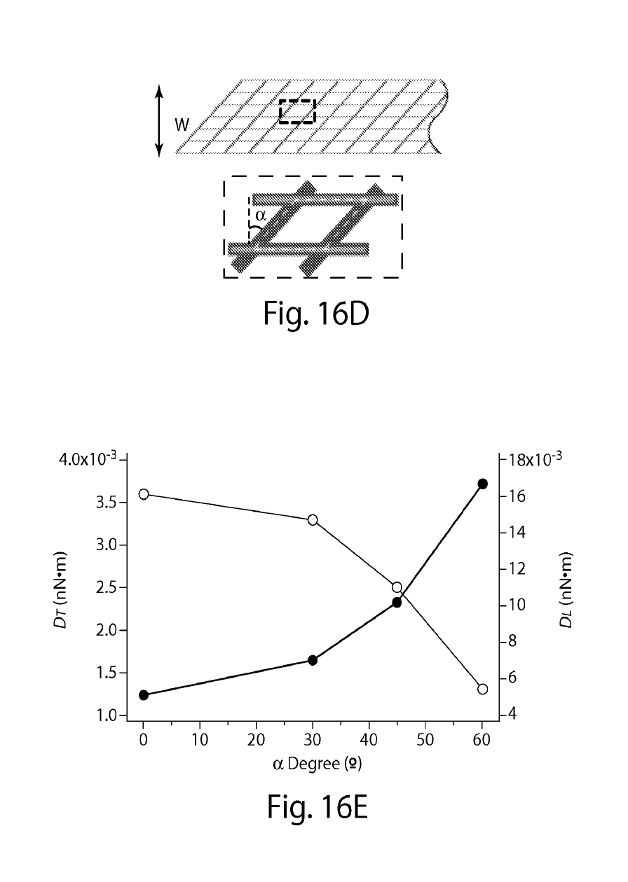

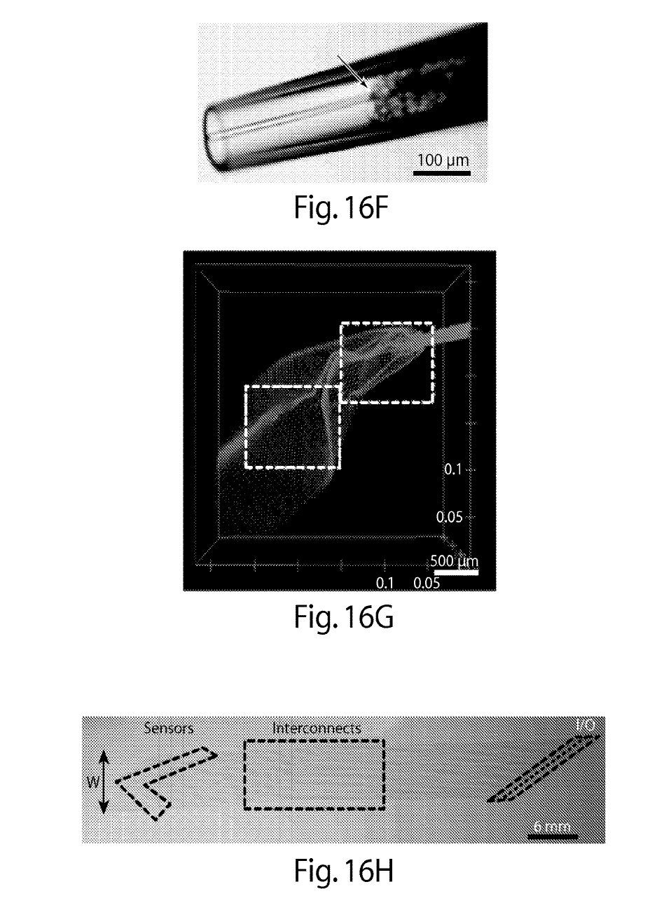

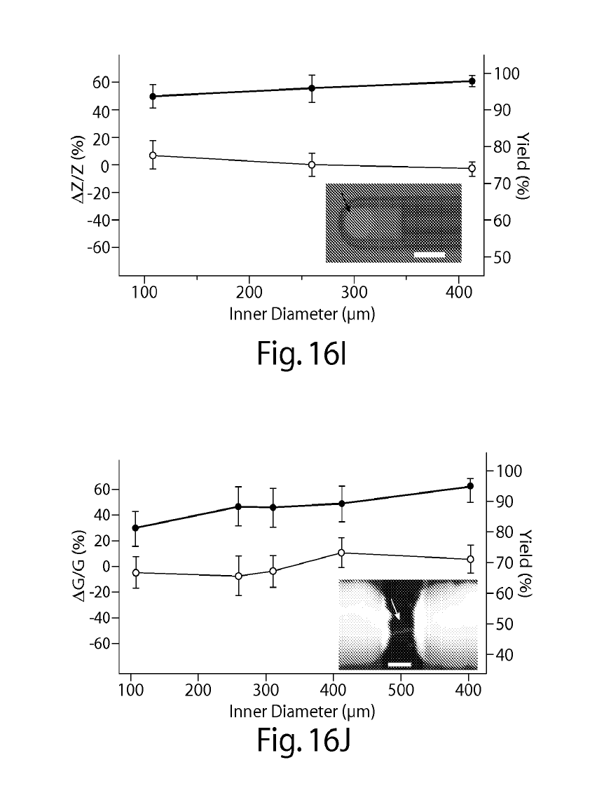

These examples describe the structural design and demonstration of macroporous flexible mesh electronics that allow electronics to be precisely delivered into 3D structures by syringe injection and subsequently relax and interpenetrate within the internal space of man-made and biological materials. Syringe injection requires release of the mesh electronics from a substrate, and moreover, sub-micron thickness so that the electronics can be driven by solution through a needle. The concept of syringe injectable electronics is shown schematically in FIGS. 16A-16C and involves (i) loading the mesh electronics into a syringe and needle, (ii) insertion of the needle into the material or internal cavity and initiation of mesh injection (FIG. 16A), (iii) simultaneous mesh injection and needle withdrawal to place the electronics through the targeted region (FIG. 16B), and (iv) delivery of the input/output (I/O) region of the mesh outside of the material (FIG. 16C) for subsequent bonding and measurements.

Design and implementation of electronics for syringe injection. Overall, the mechanical properties of the free-standing mesh electronics are important to the injection process. The basic mesh structure (FIG. 16D) includes longitudinal polymer/metal/polymer elements, which function as interconnects between exposed electronic devices and I/O pads, and transverse polymer elements. The mesh longitudinal bending stiffness, D_{L}, and the mesh transverse bending stiffness, D_{T}, are determined by the mesh unit cell and corresponding widths and thickness of the longitudinal and transverse elements, and the angle, alpha, where alpha=0° corresponds to a rectangular mesh unit cell. A simulation of D_{T} and D_{L} as a function of alpha (FIG. 1e) shows that D_{T} (D_{L}) decreases (increases) as expected for increasing alpha. For example, D_{T} decreases 30% as alpha increases from 0° to 45° (FIG. 16E) for representative mesh electronics used in these studies (structural parameters shown as per entries 1-4 of Table 1), and this (alpha=)45° value is ca. 25 times lower than the D_{T} value for a comparable total thickness (800 nm) continuous thin film. These results show that increasing alpha facilitates bending along the transverse direction (reduced D_{T}) and could allow for rolling-up of the mesh electronics within a needle constriction, while at the same time reducing bending and potential buckling along the injection (longitudinal) direction through an increase in D_{L}.

The mesh electronics were fabricated (details, see, below) and fully-released from substrates, and were then loaded into glass needles by drawing the device end of the mesh electronics from the larger end to the needle opening with suction. The needle with oriented mesh electronics was reversed, mounted on a three-axis manipulator and connected to a microinjector. Images of the injection of a 2 mm wide mesh electronics sample through a 95 micrometer ID glass needle show the compressed mesh ca. 250 micrometer from the needle opening (FIG. 16F), and then injected ca. 0.5 cm into 1× PBS solution (FIG. 16G), where the latter 3D reconstructed confocal fluorescence image highlights the unfolding of the mesh structure from the point of the needle constriction (dashed box). Higher resolution images from the region around the needle and several millimeters into solution show the continuity of the mesh structure as it unfolds in solution. Similar results were also obtained for injection of a 1.5 cm overall width mesh electronics through a 20 gauge (600 micrometer ID) metal needle demonstrating generality of this approach for injection through common glass and metal syringe needles.

To test further the electrical continuity and functionality of the mesh electronics postinjection, anisotropic conductive film (ACF) was used to connect the I/O pads of the mesh electronics post-injection to flexible cables that were interfaced to measurement electronics. Comparison of the connection resistance values obtained using a standard flip-chip bonder and custom set-up suitable for bonding in restricted environments, including in vivo measurements, shows similar values that were also comparable to reported contact resistances for ACF. Measurements of the change in electrical performance and yield of devices following injection into 1× PBS solution through ca. 100-600 micrometer ID metal needles (FIGS. 16I and 16J) highlight several points. The average device yield for metal electrochemical devices (FIG. 16I), which each used a single ca. 3 cm long metal interconnect line from I/O pad to device end, was greater than 94%. In addition, the average device impedance, which represents an important characteristic for voltage sensing applications, changed <7% post injection (FIG. 16I). Measurements of the yield of silicon nanowire field-effect transistor (FET) devices, which each required two ca. 3 cm long metal interconnect lines, was >90% for needle IDs from 260 to 600 micrometers and only dropped to 83% for the smallest 100 micrometer ID needles (FIG. 16J). The FETs also showed <12% conductance change on average post injection (FIG. 16J). Taken together, the results in this particular example demonstrate the robustness of this mesh electronics design and the capability of maintaining good device performance following injection through a wide-range of needle IDs.

FIG. 16 shows syringe injectable electronics. FIGS. 16A to 16C are schematics of injectable electronics. The lines highlight the overall mesh structure and indicate the regions of supporting and passivating polymer mesh layers and metal interconnects between I/O pads (filled circles) and recording devices (filled circles). FIG. 16D, Schematic of the mesh electronics design (upper image), where the horizontal and diagonal lines represent polymer encapsulated metal interconnects and supporting polymer elements, respectively, and W is the total width of the mesh. The dashed black box (lower image) highlights the structure of one unit cell (white dashed lines), where alpha is the angle deviation from rectangular. FIG. 16E shows a longitudinal mesh bending stiffness, D_{L}, and transverse mesh bending stiffness, D_{T}, as a function of alpha defined in FIG. 16D and 16G are images of mesh electronics injection through a glass needle, ID=95 micrometers, into 1× PBS solution. Bright-field microscopy image FIG. 16F of the mesh electronics immediately prior to injection into solution; the arrow indicates the end of the mesh inside the glass needle. 3D reconstructed confocal fluorescence image FIG. 16G recorded following injection of ca. 0.5 cm mesh electronics into 1× PBS solution. FIG. 16H is an optical image of an injectable mesh electronics structure unfolded on a glass substrate. W is the total width of the mesh electronics. The dashed polygon highlights the position of electrochemical devices or FET devices. The dashed boxes highlighted metal interconnect lines and metal I/O pads. FIGS. 16I and 16J show yields and change in properties post-injection for single-terminal electrochemical and two-terminal field-effect transistor (FET) devices. FIG. 16I, yield (upper) and impedance change (lower) of the metal electrodes from the mesh electronics injected through 32, 26 and 22 gauge metal needles. Inset: bright field image of a representative metal electrode on mesh electronics, where the sensing electrode is highlighted by an arrow. Scale Bar: 20 micrometers. FIG. 16J, yield (upper) and conductance change (lower) of silicon nanowire FETs following injection through 32, 26, 24, 22 and 20 gauge needles. Inset: scanning electron microscopy (SEM) image of a representative nanowire FET device in the mesh electronics; the nanowire is highlighted by the arrow. Scale bar: 2 micrometers.

### EXAMPLE 9

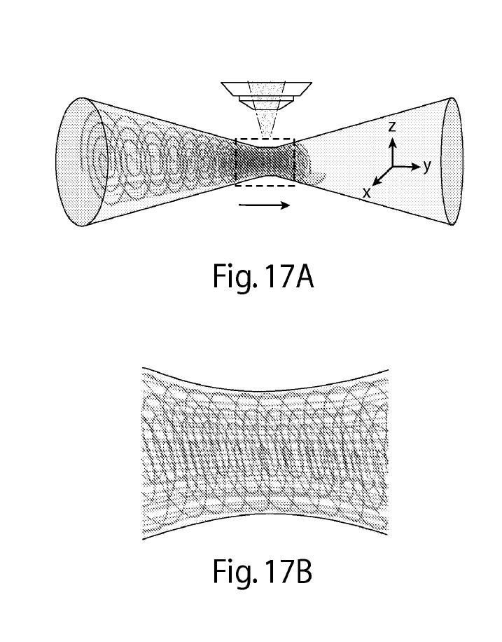

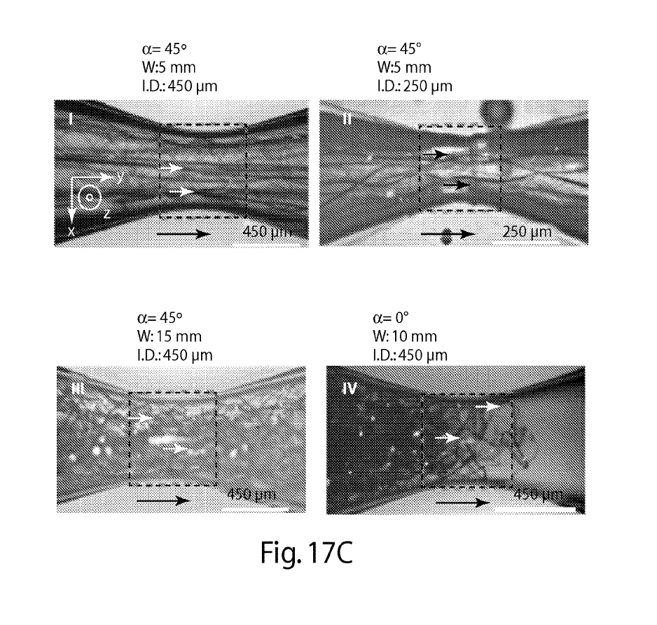

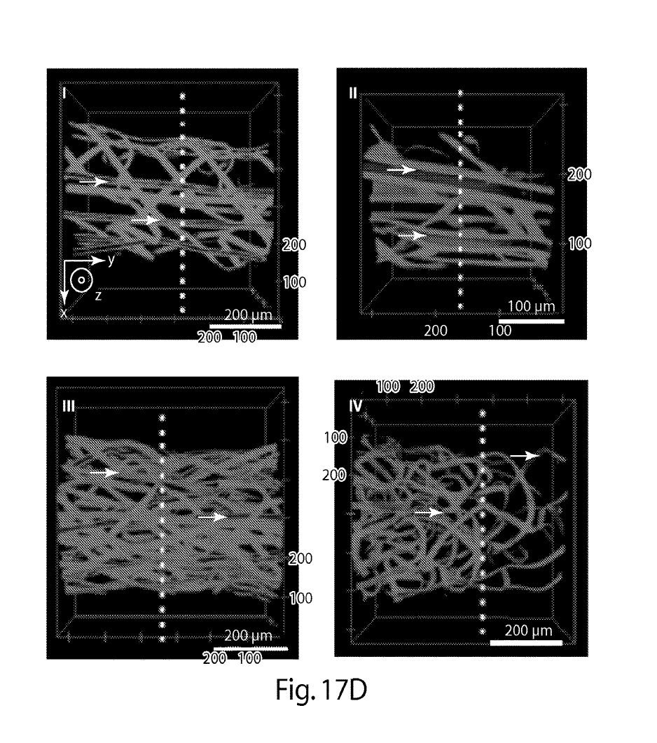

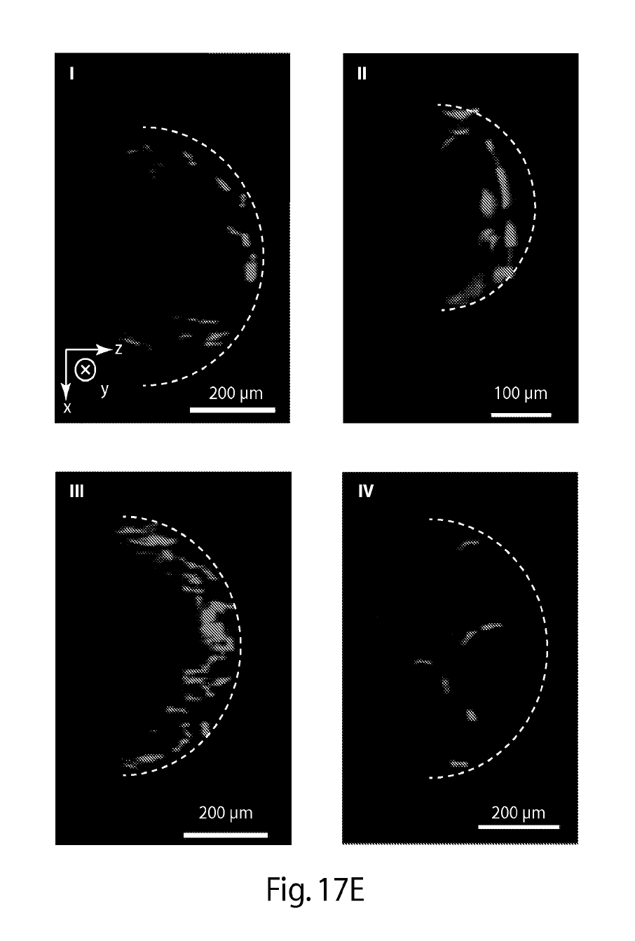

This example characterized the structures of different mesh electronics within glass needlelike constrictions to understand design parameters for successful injection. A schematic (FIGS. 17A-17B) highlights this approach in which a pulled glass tube with controlled ID central constriction was positioned under a microscope objective for bright-field and confocal fluorescence imaging, and the mesh electronics are injected partially through the constriction. Representative bright field microscopy images of mesh electronics with different structural parameters recorded from the central region of different ID glass channels (FIG. 17C) show some important features. Mesh electronics with alpha=45° and total widths substantially larger than the constriction ID colud be smoothly injected. Relatively straight longitudinal elements are seen in FIG. 17C, I and II, where the 5 mm 2D mesh widths were 11- and 20-times larger than the respective 450 and 250 micrometer ID needle constrictions. Even 1.5 cm width mesh electronics (FIG. 17C, III) can be injected smoothly through a 33-times smaller ID (450 micrometer) constriction. The density of longitudinal and transverse elements in the image made it more difficult to trace through the needle, although approximately straight longitudinal elements could still be seen.

Further insight into mesh electronics injection was obtained from higher-resolution fluorescence confocal microscopy images recorded at the same time as the above bright-field microscopy images. The corresponding 3D reconstructed confocal images of alpha=45° mesh electronics samples with mesh width/constriction ID ratios from 11 to 33 (FIG. 17D, I to III) show some important points. The longitudinal elements maintained a straight geometry without substantial bending through the constriction even for a 33:1 width: ID ratio (FIG. 17D, III). These images showed that the transverse element bend with a curvature that appeared to match the needle ID. This latter point and further structural details can be seen in cross-sectional plots of these 3D images (FIG. 17E, I to III), which showed that all of the transverse and longitudinal elements were uniformly organized near the ID of the glass constriction in tubular structures. Third, there was no evidence for fracture of alpha=45° design mesh elements in these images. Indeed, simulations of the strain versus needle ID showed that upper limit strain value for the mesh in a 100 micrometer ID needle, ˜1%, is less than the calculated critical fracture strain. Last, from a series of cross-sectional images, the longitudinal elements can be identified and the number of rolls that these mesh electronics made at the glass needle constriction were estimated as 3.4+/−0.2, 6.0+/−0.4 and 9.5+/−1.0 for FIG. 17E, I to III, respectively, which were comparable to a geometric calculation assuming that the 2D meshes roll up inside the different ID channels.

In contrast, bright-field microscopy images and 3D confocal images recorded from injection of alpha=0° mesh electronics (FIG. 17C-17E, IV) and thin-film electronics showed that these structures were not as smoothly injected through the needle-like constrictions as above. Specifically, images of a mesh electronics sample with alpha=0° (FIG. 17D, IV) but smaller width as alpha=45° (FIG. 17D, III) showed that the mesh could sometimes become jammed at the constriction. The structure of the mesh electronics was deformed and filled the cross-section of the channel versus roll-up along the ID (FIG. 17E, IV). Injection of thin film electronics with the same thickness and total width as the mesh in FIG. 17C, I for a width/needle ID ratio of 11 could sometimes become jammed in the channel. Reducing the thin film width/needle ID ratio to 4 did lead to more successful injection, although 3D confocal microscopy images also sometimes showed substantial buckling of the structure in contrast to the alpha=45° mesh electronics design. These results support the concept that reducing the transverse bending stiffness for the alpha=45° mesh design can be useful under some conditions to allow the electronics to smoothly roll-up and follow the needle ID with minimum strain and thereby allow for injection electronics with 2D widths >30-times the needle ID.

In addition, mesh electronics injection as a function of the fluid flow rate for a constant 400 micrometer I.D. needle were also investigated. It was found that smooth mesh electronics injection for flows from 20 to 150 mL/hr as long as the needle retraction speed matched the speed of the injected fluid. The lower limit for smooth injection, 20 mL/hr, is believed to be restricted by the smallest fluid drag force relative to the friction force between the rolled-up mesh electronics and the inner needle surfaces. The maximum flow, 150 mL/hr, was limited by the needle retraction speed of this set-up.

FIG. 17 shows imaging mesh electronics structure in needle constrictions. FIG. 17A is a schematic illustrating the structure of a pulled glass tube (outer shape) with mesh electronics passing from larger (left) to smallest (center) ID of tube, where the arrow indicates the direction of injection and x-y-z axes indicate coordinates relative to the microscope objective for images in FIGS. 17C to FIG. 17E. FIG. 17B is a schematic image of the mesh structure from the region of the constriction indicated by the dashed box in FIG. 17A. FIG. 17C are bright-field microscopy images of different design mesh electronics injected through glass channels. I and II, total width, W=5 mm, alpha=45° mesh electronics injected through 450 and 250 micrometer ID, respectively, glass channels. III, W=15 mm, alpha=45° mesh electronics injected through a 450 micrometer ID glass channel. IV, W=10 mm, alpha=0° mesh electronics injected through a 450 micrometer ID glass channel. The injection direction is indicated by arrows in the images; the orientation relative to the axes in FIG. 17A are indicated in I and the same for panels Ito IV. FIG. 17D, 3D reconstructed confocal images from the dashed box regions in the respective panels Ito IV in FIG. 17C; the x-y-z axes in I are the same for panels II to IV. Horizontal, small white arrows in FIGS. 17C and 17D indicate several of the longitudinal elements containing metal interconnects in the mesh electronics. FIG. 17E, cross-sectional images plotted as half cylinders from positions indicated by the vertical white dashed lines in FIG. 17D. The white dashed curves indicate the approximate IDs of the glass constrictions.

### EXAMPLE 10

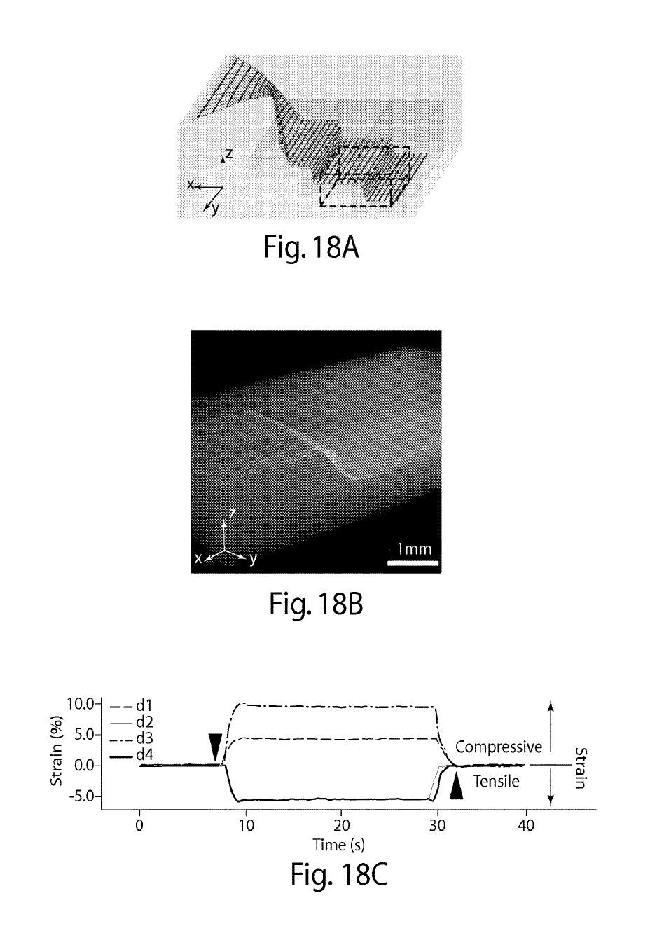

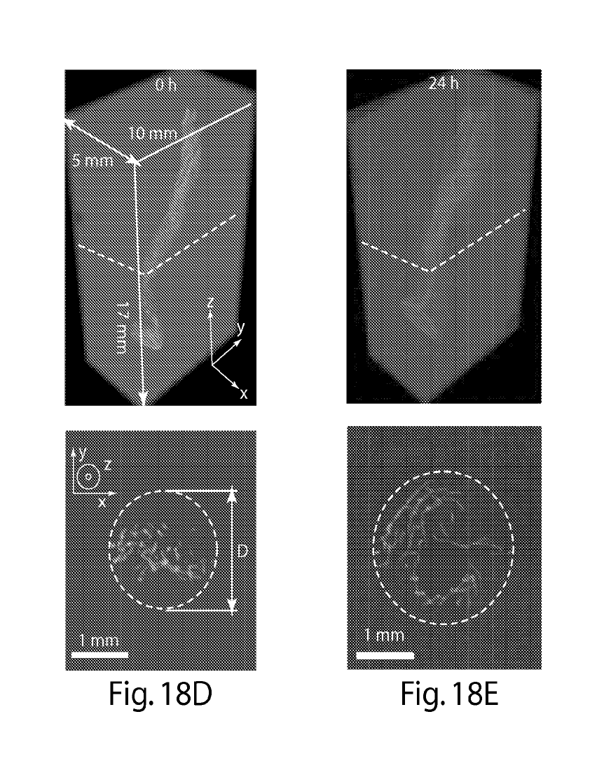

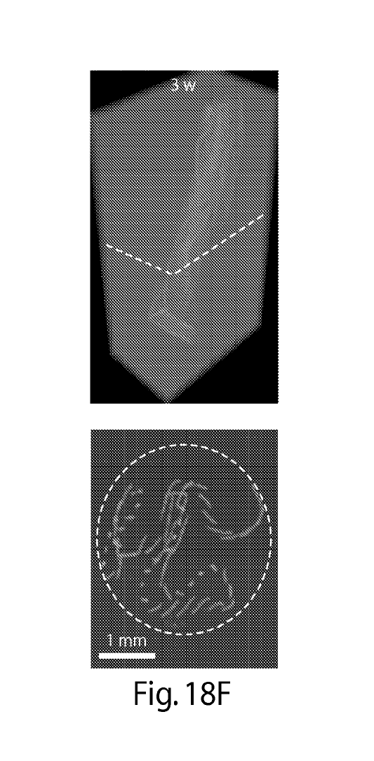

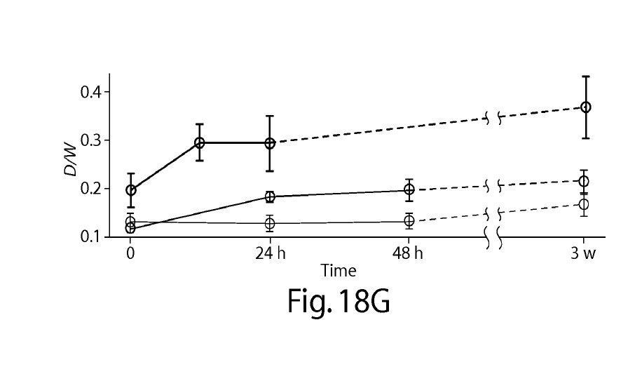

This example illustrates injection of electronics into man-made cavities and synthetic materials. This example investigated several model applications of the syringe injectable electronics, including delivery of electronics to internal regions of man-made structures and live animals. First, syringe injection and unfolding of mesh electronics into poly-dimethylsiloxane (PDMS) cavities was investigated as a technique for electrically-monitoring the internal properties of structures (FIG. 18A). The PDMS cavity was designed with a step-like internal corrugation (4 steps, 0.1 cm drop/step, and projected cavity area of 2×4.8 cm²). The mesh electronics, which incorporated addressable silicon nanowire piezoresistive strain sensors, was co-injected with diluted PDMS polymer precursors through a small injection site, with the I/O pads ejected or positioned outside the structure. Visual inspection during injection showed that the mesh electronics relaxed to ca. 80% of its 2D structure during injection and was fully-relaxed in <1 h. A micro-computed tomography image and photograph (FIG. 18B) demonstrated the unfolded mesh electronics smoothly followed the step-like internal cavity structure, and moreover, the image showed the continuity of metal interconnects in the longitudinal elements of the mesh.

After bonding a flexible cable to the external I/O pads of the mesh electronics, the response of the internal addressable silicon nanowire piezoresistive strain sensors was monitored as PDMS structures were deformed. A plot of the strain recorded simultaneously from 4 typical calibrated nanowire devices (d1-d4, FIG. 18C) as the structure that was deformed with a point load along the z-axis shows that both compressive (d1, d3) and tensile (d2, d4) local strains were recorded by the nanowires. Mapping the strain response onto the optical image of the electronics/PDMS hybrid showed the nanowire sensors were separated as far as 4 mm with 0.8 mm initial injection site. The measurements of both compressive and tensile strains were consistent with expectation for the point-like deformation of PDMS. Together with the large area strain mapping, these data suggest that syringe injection of mesh electronics with piezoresistive devices could be used to monitor and map internal strains within structural components with gaps/cracks in a manner not currently possible. More generally, the capability of nanowire devices to measure pH and other chemical changes could allow for simultaneous monitoring of corrosion and strain within internal cavities or cracks of materials and structures.

This example also investigated 3D gel structures without cavities as representative models of mesh electronics injection into soft materials and models of biological tissue. Images recorded as a function of time following injection mesh electronics into 75% Matrigel™, a tissue scaffold typically used in neural tissue engineering (FIGS. 18D to 18F) shows that the mesh unfolds ca. 80% in the radial direction over a 3-week period at 37° C. As expected, the degree of unfolding of the mesh electronics within the Matrigel™ depended on the gel concentration for fixed mesh mechanical properties (FIG. 18G); that is, a ca. 90% and 30% mesh unfolding for 25% and 100% Matrigel™ was observed, respectively, over a similar 3-week period at 37° C. The ability to inject and observe partial unfolding of the electronics within gels with tissue-like properties also suggested that co-injection with other biomaterials and/or cells could be another application direction for the injectable mesh electronics. Indeed, experiments show that coinjection of mesh electronics and embryonic rat hippocampal neurons into a Matrigel™ scaffold lead after 2 weeks culture to a 3D neural networks with neurites interpenetrating the mesh electronics. Such co-injection could be used for a variety of opportunities for tissue engineering or stem cell therapy.

FIG. 18 shows syringe injection of mesh electronics into 3D synthetic structures. FIG. 18A shows a schematic of a mesh electronics injected with uncured PDMS precursor into a PDMS cavity with I/O pads unfolded outside the cavity. The injected PDMS precursors were cured after injection. The lines highlight the overall mesh structure and indicate the regions of supporting and passivating polymers and the lighter lines indicate the metal interconnects between I/O pads (filled circle) and devices (darker filled circle). FIG. 18B is a micro-computed tomography image showing the zoomed-in structure highlighted by the black dashed box in FIG. 18A. FIG. 18C, 4 nanowire devices response to pressure applied on the PDMS. The downward and upward pointing triangles denote the times when the strain was applied and released, respectively. The downward and upward arrows show the tensile and compressive strains, corresponding to the minus and plus change of conductance, respectively. FIGS. 18D to 18F, (upper images) 3D reconstructed micro-computed tomography images of a mesh electronics injected into 75% Matrigel™after incubating for 0 h (FIG. 18D), 24 h (FIG. 18E), and 3 weeks (FIG. 18F) at 37° C. The x-y-z axes are shown in FIG. 18D and the same for FIG. 18E and FIG. 18F, where the injection direction is ca. along the z-axis. Corresponding cross-section images at z=10 mm with 500 micrometer thicknesses; the positions of the cross-sections are indicated by white dashed lines in the upper images. The maximum extent of mesh electronics unfolding was highlighted by white dashed circles with diameter, D, in each image. FIG. 18G, Time dependence of mesh electronics unfolding following injection into 25% (upper), 75% (middle) and 100% (lower) Matrigel™; the measured diameter, D, was normalized by the 2D width, W, of the fabricated mesh electronics. D was sampled from five cross-sections taken at z=5, 7.5, 10, 12.5 and 15 mm to obtain the average+/−1SD.

### EXAMPLE 11

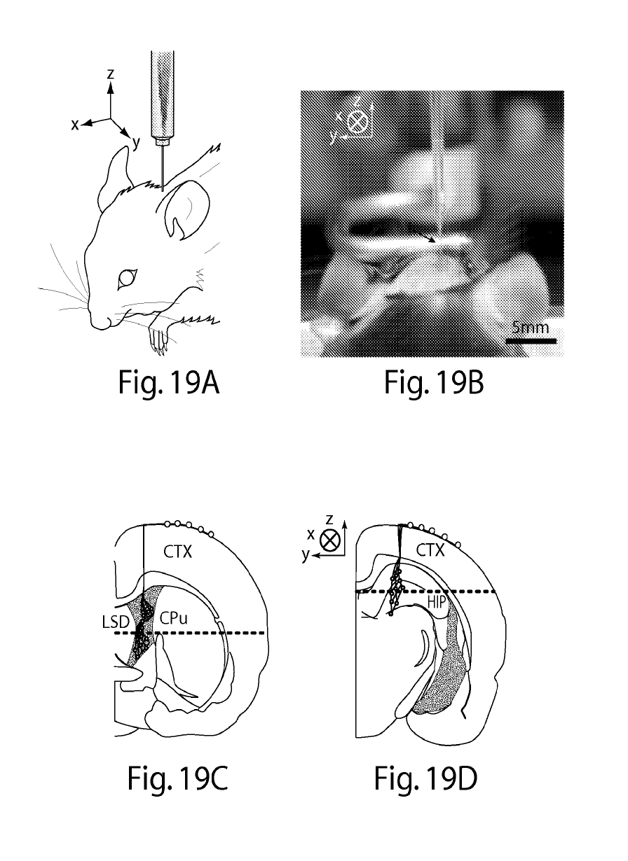

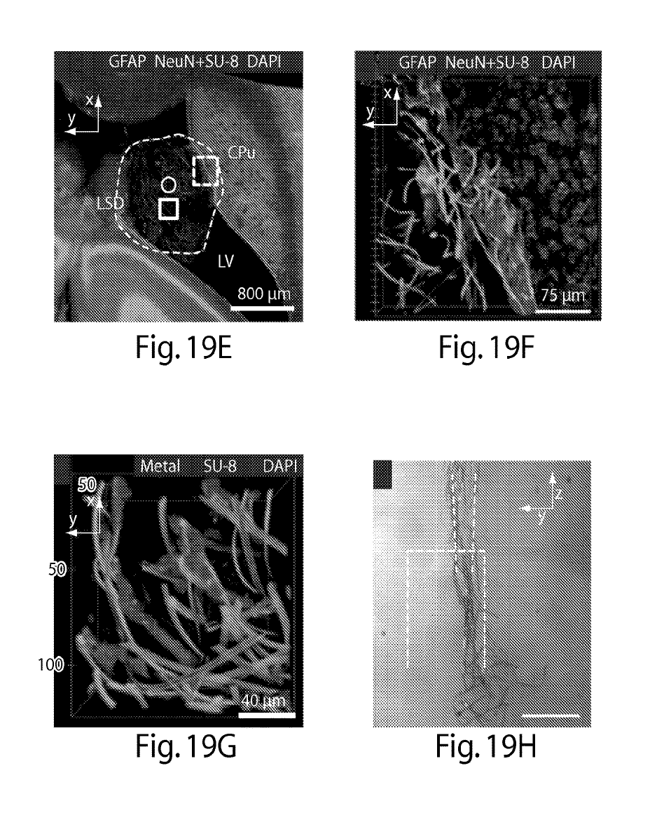

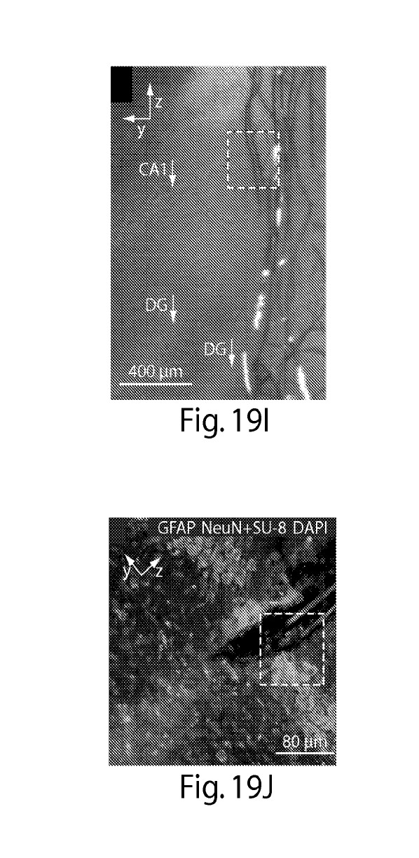

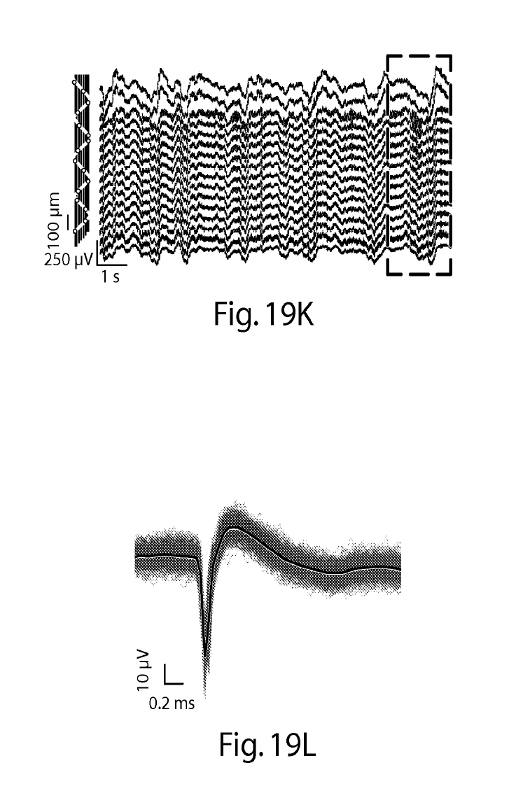

This example illustrates injection of mesh electronics into brains of live animals. In particular, this example investigated the behavior of mesh electronics injected into the brains of live rodents, where the mesh electronics were treated as biochemical reagents delivered to specific brain regions by stereotaxic injection, as shown schematically in FIG. 19A. In a typical procedure, a 100-200 micrometer ID glass needle loaded with mesh electronics, mounted in the stereotaxic apparatus and connected to a microinjector was positioned to a specific coordinate in the brain of an anesthetized mouse (FIG. 19B), and then the mesh was injected concomitantly with retraction of the needle so that the electronics is extended in the longitudinal (injection) direction. The capability of delivering millimeters width flexible electronics through 100′s micrometer outer diameter (OD) glass needles allowed for a much smaller window in the skull (e.g., <500 micrometer diameter used in these experiments) than the width of electronics thereby reducing the invasiveness of surgery. Chronic behavior was characterized 5 weeks post-injection, where electronics were delivered to both the lateral ventricle (LV) and hippocampus (HIP) regions of the brain (FIGS. 19C and 19D).

Confocal microscopy images recorded from tissue slices from the LV region prepared 5 weeks post-injection of the mesh electronics (FIGS. 19E-19G) demonstrate several important points. The mesh electronics relaxed from the initial ˜200 micrometer injection diameter to bridge the caudoputamen (CPu) and lateral septal nucleus (LSD) regions that define the boundaries of the cavity in this slice (FIG. 19E). Higher-resolution images from boundary between the mesh electronics and the CPu/subventricular zone (FIG. 19F) showed that mesh electronics could interpenetrate with the boundary cells, and moreover, that cells stained with neuron marker NeuN associated tightly with the mesh. Control image recorded from the same tissue slice at the LV region from opposite hemisphere without injected mesh electronics showed that the level of glial fibrillary acidic protein (GFAP) expression was similar with and without the injected mesh electronics. These data indicate that there is little chronic tissue response to the foreign mesh electronics. Images recorded of the mesh electronics in the middle of LV (FIG. 19G) showed a large number of 4′,6-diamidino-2-phenylindole (DAPI) stained cells were bound to the mesh structure. These images indicated that (i) the mesh expanded to integrate within the local extracellular matrix (i.e., the mesh is neurophilic), (ii) cells formed tight junctions with the mesh, and (iii) neural cells migrated 100's of microns from the subventricular zone along the mesh structure. Notably, these results suggest using injectable electronics to directly mobilize and monitor neural cells from LV region to brain injury and delivering flexible electronics to other biological cavities for recording.

In addition, mesh electronics were injected in the dense neural tissue of the HIP (FIG. 19D). Bright-field images of coronal tissues slices, prepared 5 weeks post-injection (FIG. 19H) demonstrated that the mesh electronics was fully extended in the longitudinal direction. The mesh only relaxed a small amount with respect to the initial injection diameter (dashed lines in FIG. 19H), given that the force to bend the mesh was comparable to the force to deform the tissue. In addition, an overlay of bright-field and DAPI epifluorescence images (FIG. 19I) showed that injection of the mesh electronics did not disrupt substantially the CA1 and dentate gyrus (DG) layers of this region. Notably, confocal microscopy images (FIG. 19J) highlight several characteristics. Analysis of the GFAP fluorescence intensity showed that there was a limited or an absence of astrocyte proliferation in the vicinity of the mesh, although the full image (FIG. 19J) indicated a reduction in cell density at the central region of injection. Significantly, analysis of a similar horizontal slice sample prepared from an independent mesh injection, also showed an absence of astrocyte proliferation around the electronics testifying to the robustness of this observation. These images showed many healthy neurons (NeuN signal) surrounding the SU-8 ribbons of the mesh (FIG. 19J), and fluorescence intensity analysis showed that the NeuN signal around injected mesh electronics was 1.36+/−0.26 higher than that away from electronics. These observations, which were similar to the results for injections into the LV showed the capability of the mesh electronics to promote positive cellular interactions, and are distinct from the chronic response of neural tissue from insertion of typical electrical probes where neuron density is reduced/astrocyte density increased near to conventional probes. These examples thus suggest that the injectable mesh electronics will offer substantial advantages for stable chronic recording.

These results may be attributed to the ultra-small bending stiffness and micrometer feature size of the mesh electronics delivered by syringe-injection. The bending stiffness of injected mesh electronics (0.087 nN·m) is 4-6 orders of magnitude smaller than that of previous implantable electronics such as silicon probe (4.6×10⁵ nN·m), carbon fibers (3.9×10⁴ nN·m) or thin-film electronics (0.16-1.3×10⁴ nN·m). The flexibility of the injected electronics was closer to the flexibility of tissue, which may minimize mechanical trauma caused by motion between the probe and the surrounding tissue. In addition, the feature sizes of the injected mesh electronics, 5-20 micrometers, are generally the same as single cells. Small feature sizes may be attributed to reduced chronic damage from implanted probes even when the probe stiffness is much greater than neural tissue.

Preliminary studies were also performed to verify the capability of injected mesh electronics for recording of brain activity. Mesh electronics were injected stereotaxically to the hippocampus of anesthetized mice using a procedure similar to that described above for chronic histology, and then the I/O was bonded to interface cable. Representative multichannel recording using mesh electronics with 20 micrometer diameter evaporated Pt-metal electrodes (FIG. 19K) yielded well-defined signals in all 16 channels, which also demonstrated the integrity of electronics after injection into brain tissue. The modulation amplitude, 200-400 microvolts, and dominant modulation frequency, 1-4 Hz, recorded are characteristic of microwave local field potentials (LFPs) in the anesthetized mouse. Moreover, spatiotemporal mapping of the LFP recordings revealed a characteristic hippocampal field activity for the rodent brain. In addition, sharp downward spikes were observed, and standard analysis of this data using a 300-6000 Hz bandpass filter and spike-sorting algorithm (FIG. 19I) demonstrated that these spikes displayed a uniform potential waveform with an average duration of ca. 2 ms and peak-to-peak amplitude of ca. 70 microvolts characteristic of that expected for a single-unit action potential. Importantly and in the context of long-term chronic recording, SU-8 is generally biocompatible and stable long-term, and it has been shown that metal oxide passivated silicon nanowire sensors also exhibit the long-term stability in physiological environment, thus suggesting the excellent potential of this example syringe injectable electronics for chronic implantation and recording. Significantly, it is believed these results together with the ‘neurophilic’ chronic response demonstrated in histology offer substantial promises for future investigations of long-term brain activity mapping.

FIG. 19 shows syringe injectable electronics into in vivo biological system. FIG. 19A is a schematic showing in vivo stereotaxic injection of mesh electronics into a mouse brain. FIG. 19B is an optical image of the stereotaxic injection of mesh electronics into an anesthetized 3 months old mouse brain. FIGS. 19C and 19D, Schematics of coronal slices illustrating the two distinct areas of the brain that mesh electronics were injected: FIG. 19C, through the cerebral cortex (CTX) into the lateral ventricle (LV) cavity adjacent to the caudoputamen (CPu) and lateral septal nucleus (LSD), and FIG. 19D, through the CTX into the hippocampus (HIP). Lines highlight and indicate the overall structure of mesh and dark filled circles indicate recording devices. The dashed line in FIG. 19C indicates the direction of horizontal slicing for imaging. FIG. 19E, Projection of 3D reconstructed confocal image from 100 micrometers thick, 3.17 mm long and 3.17 mm wide volume horizontal slice 5 weeks post-injection at the position indicated by dashed line in FIG. 19C. Dashed line highlights the boundary of mesh inside LV, and the solid circle indicates the size of the needle used for injection. Shading in this correspond to GFAP, NeuN/SU-8 and DAPI, respectively, and are denoted at the top of the image panel in this and subsequent images. FIG. 19F, 3D reconstructed confocal image at the interface between mesh electronics and subventricular zone (SVZ). FIG. 19G, 3D reconstructed confocal image at the ca. middle (of x-y plane) of the LV in the slice. FIG. 19H, bright-field microscopy image of a coronal slice of the HIP region 5 weeks post-injection of the mesh electronics at the position indicated in FIG. 19D. Dashed lines indicate the boundary of the glass needle. The white arrows indicate longitudinal elements that were broken during tissue slicing. Black dashed lines indicate the boundary of each individual image. FIG. 19I, overlaid bright field and epi-fluorescence images from the region indicated by white dashed box in FIG. 19H. Shading corresponds to DAPI staining of cell nuclei, white arrows indicate CA1 and dentate gyrus (DG) of the HIP. FIG. 19J, projection of 3D reconstructed confocal image from 30 micrometer thick, 317 micrometer long and 317 micrometer wide volume from the zoomed-in region highlighted by the black dashed box in i. FIG. 19K, Acute in vivo 16-channel recording using mesh electronics injected into a mouse brain. The devices were Pt-metal electrodes (impedance ˜950 kiloohms at 1 kHz) with their relative positions marked by spots in the schematic (left panel), and the signal was filtered with 60 Hz notch during acquisition. FIG. 19L, superimposed single-unit neural recordings from one channel after 300-6000 Hz band-pass filtering. The line represents the mean waveform for the single-unit spikes.

In summary, these examples show a new strategy for delivery of electronics to the internal regions of 3D man-made and biological structures that involves syringe injection of submicron thickness, large-area macroporous mesh electronics inside. Mesh electronics with 2D widths at least 30-times the needle ID can be injected and a high yield of active electronic devices can be maintained. In-situ imaging and modeling showed that the optimized transverse/longitudinal stiffness enables the mesh to ‘roll-up’ passing through needle constrictions. It was demonstrated that injected mesh electronics with addressable piezo-resistive devices were capable of monitoring internal mechanical strains within bulk structures, and it has also been shown that mesh electronics injected into the brains of mice exhibited little chronic immuno-reactivity, which indicate the injected mesh electronics are neurophilic, and can reliably monitor brain activity. Compared to other delivery methods, this syringe injection approach allows delivery of large (with respect to injection opening) flexible electronics into cavities and existing synthetic materials through a small injection site and a relatively rigid shell. Moreover, with subsequent self-unfolding of the rolled-up structure, injected electronics can fill the internal space of the cavities and materials that exhibit viscoelastic behavior.

### EXAMPLE 12

Following are additional materials and methods used in the above examples. Generally, freestanding injectable mesh electronics were fabricated on nickel relief layers. See, e.g., U.S. Pat. Apl. Pub. Nos. 2014/0073063 and 2014/0074253 and Int. Pat. Apl. Pub. No. WO 2014/165634, each incorporated herein by reference in its entirety. Following release from the substrate, mesh electronics were modified by poly-Dlysine (MW 70,000-150,000, Sigma-Aldrich Corp.) and then loaded into syringe fitted with either a metal needle or a glass needle pulled by the commercial available pipette puller (Model P-97, Sutter Instrument). A microinjector (NPIPDES, ALA Scientific instruments Inc.) and manually controlled syringes (Pressure Control Glass Syringes, Cadence, Inc.) were used to inject electronics. Confocal microscopes (Olympus Fluoview FV1000 and Zeiss LSM 780 confocal microscope) were used to image the structure of the mesh electronics in glass channels and immunostained mouse brain slices. ACF (AC-4351Y, Hitachi Chemical Co.) bonding to the mesh electronics I/O was carried out using a home-made or commercial bonding system (Fineplacer Lambda Manual Sub-Micron Flip-Chip Bonder, Finetech, Inc.) with a flexible cable (FFC/FPC Jumper Cables PREMO-FLEX, Molex). The strain response of silicon nanowire piezoresistive strain sensors was measured by a multi-channel current/voltage preamplifier (Model 1211, DL Instruments, Brooktondale, N.Y.), filtered with a 3 kHz low pass filter (CyberAmp 380, Molecular Devices), and digitized at a 1 kHz sampling rate (AxonDigi1440A, Molecular Devices), with a 100 mV DC source bias voltage. For in vivo brain recording from metal electrodes, the flexible cable was connected to a 32-channel Intan RHD 2132 amplifier evaluation system (Intan Technologies LLC., Los Angeles, Calif.) with an Ag/AgCl electrode acting as the reference. A 20 kHz sampling rate and 60 Hz notch were used during acute recording.

Open mesh electronics. The overall structure and relevant parameters of the macroporous mesh electronics include the following. W, the total mesh width; w₁, width of longitudinal ribbons along injection/long axis of mesh, w₂, width of transverse ribbons, that cross and connect to the longitudinal ribbons with an angle, alpha, relative to the longitudinal ribbons; L₁, the mesh unit cell length in the longitudinal direction; L₂, the mesh unit cell length in the transverse direction; and wₘ, the width of metal lines, which run along the longitudinal ribbons. The longitudinal and transverse ribbon widths ranged from 5-40 micrometers, and alpha was 45° or 0° . The embedded metal (SU-8/metal/SU-8) interconnects run along longitudinal ribbons; the metal contacts to nanowire transistor and bend-up passive metal sensors also have a metal line component embedded in the transverse ribbons.

Thin film electronics. Control samples with the same thickness as the mesh electronics but comprising a standard flexible thin-film structure were also designed and fabricated. The metal line patterns, thickness and widths are the same as design the tilted mesh electronics. The overall widths, W, of thin film electronics were 0.1-5 mm.

Free-standing mesh electronics fabrication, initial fabrication steps. The overall fabrication of the syringe injectable electronics is based on methods described previously. See U.S. Pat. Apl. Pub. Nos. 2014/0073063 and 2014/0074253 and Int. Pat. Apl. Pub. No. WO 2014/165634, each incorporated herein by reference in its entirety. Steps include: (1) 100 nm nickel metal, which serves as a final relief layer, was deposited on the silicon fabrication substrate (600 nm SiO₂, n-type 0.005 ohm cm, Nova Electronic Materials, Flower Mound, Tex.) by thermal evaporation; (2) A 300 to 400 nm layer of SU-8 photoresist (2000.5; MicroChem Corp., Newton, Mass.) was spin-coated on the fabrication substrate, prebaked (65° C./2 min; 95° C./2 min), and then (3) patterned by photolithography to define the bottom SU-8 layer of the injectable mesh electronics structure. (4) After post baking (65° C./2 min; 95° C./2 min), and developing by SU-8 Developer (MicroChem Corp., Newton, Mass.), the SU-8 pattern was cured at 180° C. for 20 min. At this point, either of two distinct types of device elements, silicon nanowire transistors or passive metal electrodes, was integrated in the fabrication process; these are described separately, followed by common steps used to complete fabrication of the free-standing mesh electronics.

Nanowire transistor elements. (5a) A 300 to 400 nm layer of SU-8 photoresist was deposited on the fabrication substrate, prebaked (65° C./2 min; 95° C./4 min), and then (5b) silicon nanowires were aligned on the SU-8 layer by contact printing. (5c) Photolithography was used to define the nanowire device regions, and after post-baking (65° C./2 min; 95° C./2 min), the pattern was developed by SU-8 Developer washed with isopropanol (2 times, 30 s per wash) to remove nanowires outside of the device regions. (5d) The new SU-8 pattern was cured at 180° C./20 min. (5e) Nanowire device element contacts were then fabricated. Briefly, the substrate was coated with 300 nm LOR 3A and 500 nm S1805 (MicroChem Corp., Newton, Mass.) double layer resist and patterned by photolithography. Sequential Cr/Pd/Cr (1.5/50-80/1.5 nm) metal layers were deposited by thermal evaporation followed by metal lift-off in Remover PG (MicroChem Corp., Newton, Mass.) to define the minimally-stressed nanowire contacts.

Metal electrode elements. (6a) The substrate was spin-coated with LOR 3A and S1805 double layer resist with similar thicknesses as described above. (6b) 20 micrometer diameter sensor pads (Cr/Pt, 5/50 nm) were defined by photolithography and electron beam evaporation followed by metal lift-off in Remover PG. (6c) The substrate was then spin-coated with LOR 3A and S1805 double layer resist with similar thicknesses as described above again. (6d) For sensors designed to bend-out from the mesh plane, nonsymmetrical Cr/Pd/Cr (1.5/50-80/30-50 nm) metal lines (200 micrometers long) were patterned by photolithography and subsequent thermal deposition followed by metal lift-off in Remover PG.

Completion of free-standing mesh electronics fabrication. (7) The substrate was coated with LOR 3A and S1805 double layer resist with similar thicknesses as described above and patterned by photolithography. Unstressed, symmetrical Cr/Au/Cr (1.5/50-100/1.5 nm) metal lines were sequentially deposited followed by metal lift-off in Remover PG to define the minimally stressed interconnects/address lines. All metal lines were defined such that they are on top of and smaller in width than the SU-8 mesh pattern described in steps 1-5. (8) A 300 to 400 nm layer of SU-8 photoresist was deposited on the fabrication substrate, pre-baked (65° C./2 min; 95° C./2 min), and then patterned by photolithography to match the lower SU-8 mesh structure and serve as top encapsulating/passivating layer of the metal contacts/interconnects (except for active device regions). The structure was post-baked, developed, and cured as described above. (9) In the case of nanowire transistor devices, 300 and 500 nm thick layers of LOR 3A and S1805 photoresist were deposited and defined by photolithography to protect the device region during release of the mesh from the fabrication substrate. (10) The syringe injectable mesh electronics were released from the substrate by etching the nickel layer (40% FeCl₃:39% HCl:H₂O=1:1:20) for 3-4 hours at 25° C. and then transferred to deionized (DI) water by glass pipette (5 mL, Disposable Pasteur Pipets, Lime Glass, VWR International, LLC, Radnor, Pa.). (12) The photoresist protection was removed from nanowire device meshes by exposure to ultraviolet light (430 nm, 120 s) and immersion in developer solution (MF-CD-26, MicroChem Corp., Newton, Mass.).

Injection of electronics. Surface modification of mesh electronics for aqueous injection. Freestanding mesh electronics structures were transferred by glass pipette sequentially to (a) DI water for 5 min., (b) aqueous poly-D-lysine (PDL, 0.5-1.0 mg/ml, MW 70,000-150,000, Sigma-Aldrich Corp., St. Louis, Mo.) solution for 2-12 hours at 25° C., and (c) 1× PBS (HyClone™ Phosphate Buffered Saline, Thermo Fisher Scientific Inc., Pittsburgh, Pa.) at 25° C. for storage (time limited for storage: 1-2 days).

Glass needles for injection and imaging. Glass needles for injection and imaging were prepared by using a commercial pipette puller (Model P-97, Sutter Instrument, CA). To prepare channels for imaging, the pulling was halted and suspended in the middle without breaking the glass tube. The channel sizes were characterized by confocal fluorescence microscopy, where rodamine-6G (Sigma-Aldrich Corp., St. Louis, Mo.) solution was filled into the channel for imaging. For a channel inner diameter (ID) smaller than 300 micrometers, epoxy glue was used to increase stability during imaging. Clean-cut needles were prepared by scoring (#CTS, Sutter Instrument, CA) and mechanical breakage followed by optical microscopy examination. To introduce the mesh electronics into glass needles, the tip end of a glass needle was connected to a syringe, and then the large end of the glass needle was used to suck the mesh electronics in towards the sharp needle tip. The correct orientation of the mesh electronics (i.e., recording devices at the needle tip) is readily achieved given visual asymmetry of the structures. The glass needle was removed from the plastic tube/syringe and the large end connected to a conventional micropipette holder (Q series holder, Harvard Apparatus, Holliston, Mass.). A microinjector was connected to this holder by plastic tubing. The injection process was controlled using a microinjector (NPIPDES, ALA Scientific instruments Inc., Farmingdale, N.Y.); for example, the injection length per microinjector pulse can yield well-defined ejection of the mesh electronics from the needles.

Injection through metal needles. After surface modification, the mesh electronics was transferred by glass pipette into a syringe (Pressure Control Glass Syringes, Cadence, Inc., Cranston, R.I.) fitted with a metal needle (18-32 gauge, Veterinary Needles, Cadence, Inc., Cranston, R.I.). The syringe was assembled and the plunger carefully pressed to drive the region containing devices into the needle, and then to inject the mesh into aqueous solutions.

Input/output (I/O) bonding with anisotropic conductive film (ACF). The I/O connection pads at the end of the mesh electronics structure were bonded to a flexible cable post-injection for measurements. First, the I/O region was allowed to unfold in solution layer outside of the injected materials, and then rinsed with ethanol and dried. Second, a piece of ACF (ACF, CP-13341-18AA, Dexerials America Corporation, San Jose, Calif.), 1.5 mm wide and 15 mm long was over the I/O pads and partially bonded for 10 sec at 75° C. and 1 MPa using a homemade or commercial bonder (Fineplacer Lambda Manual Sub-Micron Flip-Chip Bonder, Finetech, Inc., Manchester, N.H.). Third, a flexible cable (FFC/FPC Jumper Cables PREMO-FLEX, Molex, Lisle, Ill.) was placed on the ACF, aligned with I/O pads and bonded for 1-2 min at 165-200° C. and 4 MPa.

Injection of mesh electronics, co-injection into polymer cavities with a polymer precursor. Cavities for injection were formed from two pieces of cured polydimethylsiloxane (PDMS, Sylgard 184, Dow Corning Corporation, Midland, Mich.). Steps for the co-injection include the following: (1) mesh electronics were transferred from DI water to ethanol after etching. (2) PDMS pre-polymer components were prepared in a 10:1 (base:cure agent; Sylgard 184, Dow Corning Corporation, Midland, Mich.), diluted by hexane 1:3 PDMS:hexane volume ratio, and then (3) the mesh electronics was transferred to the PDMS/hexane solution and the resulting homogeneous suspension loaded into a glass syringe. (4) The device region of mesh was injected through a 16 or 18 gauge metal needle into the cavity, and the I/O region was positioned outside the cavity on a silicon wafer or glass slide. (5) The I/O region was washed with hexane to remove PDMS residue and bonded to a flexible cable interface as described above. The PDMS cavity with the mesh electronics was left at room temperature for 2-4 hours to allow for evaporation of hexane, and then undiluted PDMS precursors were injected into the cavity to fill the entire volume and cured at room temperature for 48 h.

Injection into Matrigel™. PDL modified mesh electronics were transferred to 1× PBS solution, autoclaved for 1 hour, transferred into Neurobasal™ medium (Invitrogen, Grand Island, N.Y.) by glass pipette, and then loaded into glass syringe as described above. 100% Matrigel™ (BD Bioscience, Bedford, Mass.) alone or diluted with Neurobasal™ medium to 75 and 25% (v/v) was polymerized for 20 min at 37° C. in an incubator. Mesh electronics were injected into the 100, 75 and 25% polymerized Matrigel™ samples, and the hybrid structures were incubated at 37° C. and imaged (FIGS. 18D-18F) at different times to investigate mesh unfolding in the gel.

Co-injection of mesh electronics with neurons. Hippocampal neurons (Gelantis, San Diego, Calif.) were prepared using a standard protocol. In brief, 5 mg of NeuroPapain Enzyme (Gelantis, San Diego, Calif.) was added to 1.5 ml of NeuroPrep Medium (Gelantis, San Diego, Calif.). The solution was kept at 37° C. for 15 min, and sterilized with a 0.2 micrometer syringe filter (Pall Corporation, Mich.). Day 18 embryonic Sprague/Dawley rat hippocampal tissue with shipping medium (E18 Primary Rat Hippocampal Cells, Gelantis, San Diego, Calif.) was spun down at 200 g for 1 min. The shipping medium was exchanged for NeuroPapain Enzyme medium. A tube containing tissue and the digestion medium was kept at 30° C. for 30 min and manually swirled every 2 min, the cells were spun down at 200 g for 1 min, the NeuroPapain medium was removed, and 1 ml of shipping medium was added. After trituration, cells were isolated by centrifugation at 200 g for 1 min, and then resuspended in 5-10 mg/ml Matrigel™ at 4° C. Matrigel™ with neurons were mixed with electronics at 4° C. and then loaded into syringe with metal gauge needle. The electronics and neurons were co-injected into 30% (v/v) polymerized Matrigel™ in culture plate and then placed in incubator to allow Matrigel™ to gel at 37° C. for 20 min. Then 1.5 ml of NeuroPure plating medium was added. After 1 day, the plating medium was changed to Neurobasal™ medium (Invitrogen, Grand Island, N.Y.) supplemented with B27 (B27 Serum-Free Supplement, Invitrogen, Grand Island, N.Y.), Glutamax™ (Invitrogen, Grand Island, N.Y.) and 0.1% Gentamicin reagent solution (Invitrogen, Grand Island, N.Y.). The in-vitro co-cultures were maintained at 37° C. with 5% CO₂ for 14 days, with medium changed every 4-6 days. After incubation, cells were fixed with 4% paraformaldehyde (Electron Microscope Sciences, Hatfield, Pa.) in PBS for 15-30 min, followed by 2-3 washes with ice-cold PBS. Cells were pre-blocked and permeabilized (0.2-0.25% Triton X-100 and 10% feral bovine serum (F2442, Sigma-Aldrich Corp. St. Louis, Mo.) for 1 hour at room temperature. Next, the cells were incubated with primary antibodies Anti-neuron specific beta-tubulin (in 1% FBS in 1% (v/v)) for 1 hour at room temperature or overnight at 4° C. Then cells were incubated with the secondary antibodies AlexaFluor-546 goat anti-mouse IgG (1:1000, Invitrogen, Grand Island, N.Y.).

In vivo rodent brain injection. Mouse preparation. (1) Adult (25-35 g) male C57BL/6J mice (Jackson lab) and Adult (25-35 g) male transgenic mice FVB/N-Tg (GFAPGFP)14Mes/J (Jackson lab) were group-housed, given access to food pellets and water ad libitum and maintained on a 12 h: 12 h light: dark cycle. (2) All animals were held in a facility beside lab 1 week prior to surgery, post-surgery and throughout the duration of the behavioral assays to minimize stress from transportation and disruption from foot traffic. All procedures were approved by the Animal Care and Use Committee of Harvard University and conformed to US National Institutes of Health guidelines.

Stereotaxic surgery. (3) After animals were acclimatized to the holding facility for more than 1 week, they were anesthetized with a mixture of 60 mg/kg of ketamine and 0.5 mg/kg medetomidine (Patterson Veterinary Supply Inc., Chicago, Ill.) administered intraperitoneal injection, with 30 microliter update injections of ketamine to maintain anesthesia during surgery. A heating pad (at 37° C.) was placed underneath the body to provide warmth during surgery. Depth of anesthesia was monitored by pinching the animal's feet periodically. (4) Animals were placed in a stereotaxic frame (Lab Standard Stereotaxic Instrument, Stoelting Co., Wood Dale, Ill.) and then (5) a 1 mm longitudinal incision was made, and the skin was resected from the center axis of the skull, exposing a 2 mm by 2 mm portion of the skull. (6) A 0.5 mm diameter hole was drilled into the frontal and parietal skull plates using a dental drill (Micromotor with On/Off Pedal 110/220, Grobet USA, Carlstadt, N.J.). (7) The dura was incised and resected. Sterile 1× PBS was swabbed on the brain surface to keep it moist throughout the surgery. A stereotaxic arm was used to hold and position the needle containing the injectable mesh electronics.

Stereotaxic injection. (8) Mesh electronics were autoclaved for 1 hour in 1× PBS solution before injection, and then transferred into Neurobasal™ medium and loaded into the autoclaved glass needle as described above. (9) The glass needle (with diameter of 100-200 micrometrs) was mounted to a micropipette setup for injection. (10) The needle was lowered into the exposed brain surface approximately 1-2 mm into the skull (Interaural: 6.16 mm, Bregma: −3.84 mm) to test the effects of deep brain and superficial layer injections. A syringe or microinjector was used to inject the mesh electronics into the brain. The needle was retracted during injection using a linear translational stage on the stereotaxic frame. The mesh is injected concomitantly with retraction of the needle so that the electronics is extended in the longitudinal (injection) direction. (11) After injection, the needle was withdrawn from the brain tissue and the I/O region was ejected on the surface of the skull and recording scaffold.

Acute recording. (12) A ceramic plate/scaffold with a 0.5-1 cm diameter hole was fixed above the mouse brain, and (13) silicone elastomer (World Precision Instruments Inc., Sarasota, Fla.) was used to seal the gap between the mouse skull and the scaffold to form a chamber that was kept filled with 1× PBS solution. (14) After injection of electronics as described in steps 10-11, the I/O region of electronics was unfolded on the surface of the ceramic scaffold. (15) I/O pads were bonded to a flexible cable by ACF as described above. (16) A 32-channel Intan RHD 2132 amplifier evaluation system (Intan Technologies LLC., Los Angeles, Calif.) was used for acute electrophysiology recording with an Ag/AgCl electrode acting as the reference. A 20 kHz sampling rate and 60 Hz notch were used during acute recording. A 300-6000 Hz band-pass filter was applied to original recording data for single-unit spikes analyses. Superposition of single-unit spikes was conducted by Clampfit (Molecular Devices, Sunnyvale, Calif.).

Chronic testing. (17) After injection, the skin that was retracted from the center axis was replaced and the incision was sealed with C&B-METABOND (Cement System, Parkell, Inc., Edgewood, N.Y.). (18) Antiinflammatory and anti-bacterial ointment was swabbed onto the skin after surgery. A 0.3 mL intraperitoneal injection of Buprenex (Patterson Veterinary Supply Inc. Chicago, Ill., diluted with 0.5 ml of PBS) was administered at 0.1 mg/kg to reduce post-operative pain. (19) Animals were observed for 4 hours after surgery and hydrogel was provided for food, and heating pad was on at 37° C. for the remainder of post-operative care. All procedures complied with the United States Department of Agriculture guidelines for the care and use of laboratory animals and were approved by the Harvard University Office for Animal Welfare.

Incubation and behavioral analysis. (20) Animals were cared every day for 3 days after the surgery and every other day after the first 3 days. (21) Animals were administered 0.3 mL of Buprenex (0.1 mg/kg, diluted with 0.5 mL 1× PBS) every 12 hours for 3 days. Animals were also observed every other day for behavioral changes. Animals, which were surgically operated on, were housed individually in cages with food and water ad libitum. The room was maintained at constant temperature on a 12-12 h light-dark cycle.

Brain tissue preparation for chronic immunostaining. Steps for brain tissue immunostaining are as follows: (1) 4-5 weeks after the surgery, mice underwent transcardial perfusion (40 mL 1× PBS) and were fixed with 4% formaldehyde (Sigma-Aldrich Corp., St. Louis, Mo., 40 mL). (2) Mice were decapitated and brains were removed from the skull and set in 4% formaldehyde for 24 hours as post fixation and then 1× PBS for 24 hours to remove excess formaldehyde. The mesh electronics remained inside the brain throughout fixing process. (3a) For samples with mesh electronics injected in the cortex/hippocampus region, brains were blocked, separated into the two hemispheres, and (3b) mounted on the vibratome stage (Vibrating Blade Microtome Leica VT1000 S, Leica Microsystems Inc. Buffalo Grove, Ill.). (3c) 50-100 micrometer thick vibratome tissue slices (horizontal and coronal orientations) were prepared for staining. (4a) For samples with mesh electronics injected in lateral ventricle, brains were blocked and then fixed in 1% (w/v) agarose type I-B (Sigma-Aldrich Corp., St. Louis, Mo.) to fix the position of mesh electronics in the lateral ventricle cavity and then (4b) mounted on the vibratome stage. (4c) 100 μm thick vibratome tissue slices (horizontal orientations) were prepared. Coronal slices allowed for cutting in a direction along the long axis of the injection on the frontal plane and horizontal slices allowed for cuts in a direction perpendicular to the long axis of injection. (5a) Sample prepared for cryosectioning were transferred to sucrose solution (30%) overnight, and then (5b) transferred to Cryo-OCT compound (VWR, International, LLC, Chicago, Ill.) with frozen at −80° C. (5c) Frozen samples were mounted on the stage of a Leica CM1950 cryosectioning instrument (Leica Microsystems Inc., Buffalo Grove, Ill.) and sectioned into 10 micrometer thick horizontal slice.

Immunostaining. (6) Slices >30 micrometer thick were then cleared with 5 mg/mL sodium borohydride in HEPES-buffered Hanks saline (HBHS, Invitrogen, Grand Island, N.Y.) for 30 minutes, with 3-times following HBHS washes at 5-10 minute intervals. Sodium azide (4%) diluted 100× in HBHS was included in all steps. (7) Slices were incubated with 0.5% (v/v) Triton X-100 in HBHS for 30 min at room temperature. (8) All slices were blocked with 5% (w/v) FBS and incubated overnight at room temperature. (9) Slices were washed four times, 30 min intervals, with HBHS to clear any remaining serum in the tissue. (10) Slices were then incubated overnight at room temperature with the glial fibrillary acidic protein (GFAP) primary antibody (targeting astrocytes, 1:1000, #13-0300 Invitrogen, Grand Island, N.Y.) and/or NeuN primary antibody (targeting nuclei of neurons, 1:200, #ab77315 AbCam, Cambridge, Mass.) containing 0.2% triton and 3% serum. (11) After incubation, slices were washed 4-times for 30 min with HBHS. Slices were incubated with secondary antibody (1:200; Alexa Flour® 546 goat anti-rat secondary antibody, 1:200, Alexa Fluor® 488 goat anti-rabbit secondary antibody and/or 1:200, Alexa Fluor® 647 goat anti-chicken secondary antibody (for GFP labeled mice), Invitrogen, Carlsbad, Calif.) and counterstained with Hoechst 33342 (nuclein stain 1:150, #46C3-4, Invitrogen, Carlsbad, Calif.) with 0.2% Triton and 3% serum overnight. (12) After the final washes (4 times, 30 min each with HBHS), slices were mounted on glass slides with coverslips using Prolong Gold (Invitrogen, Carlsbad, Calif.) mounting media. The slides remained covered (protected from light) at room temperature, allowing for 12 hours of clearance before imaging. When the antibody solutions were first prepared, they included 0.3 Triton X-100 and 5% FBS.

Structure characterization: scanning electron microscopy (SEM, Zeiss Ultra55/Supra55VP field-emission SEMs) was used to characterize the mesh electronics structures. Confocal, bright-field and epi-fluorescence imaging was carried out using an Olympus Fluoview FV1000 confocal laser scanning microscope or Zeiss LSM 780 confocal microscope (Carl Zeiss Microscopy, Thornwood, N.Y.). Confocal images were acquired using 405, 473 and 559 nm wavelength lasers to excite components labeled with Hoechst 33342, Alexa Flour® 488, Alexa Flour® 546, GFP, and Rodamine-6G fluorescent dyes. A 635 nm wavelength laser was used for imaging Alexa Flour® 647, and imaging metal interconnects in reflective mode. Epi-fluorescence images were acquired using a mercury lamp together with standard DAPI (EX:377/50,EM:447/60), GFP (EX:473/31,EM520/35) and TRITC (EX:525/40,EM:585/40) filters. ImageJ (ver. 1.45i, Wayne Rasband, National Institutes of Health, USA) was used for 3D reconstruction and statistical analysis of the confocal images, and overlapping epi-fluorescence images and bright-field images.

Imaging of mesh electronics in glass channels. Mesh electronics and thin film control samples with different width and structure were injected into the glass channels following the same injection process described above except that process was stopped so that the mesh remained in part in the constriction of the “needle.” Confocal fluorescence microscopy was used to image the 3D structure of mesh electronics and thin films in different diameter glass needles. 3D reconstructed images were obtained using ImageJ. Cross-section images of the samples were obtained using ImageJ to re-slice 3D reconstructed images in transverse direction with 1 micrometer steps along the longitudinal direction.

Micro-computed tomography. Structures of injected mesh electronics cured in PDMS and Matrigel™ were imaged using a HMXST Micro-CT X-ray scanning system with a standard horizontal imaging axis cabinet (model: HMXST225, Nikon Metrology, Inc., Brighton, Mich.). Typical imaging parameters for electronics in PDMS were 75 kV acceleration voltage and 120 microamp electron beam current; for electronics in Matrigel™, 80 kV acceleration voltage and 130 microamp electron beam current were used. In both cases, shading correction and bad pixel correction were applied before scanning to adjust the X-ray detector; no filter was applied. CT Pro (ver. 2.0, Nikon-Metris, UK) was used to calibrate centers of Micro-CT images. VGStudio MAX (ver. 2.0, Volume Graphics GMbh, Germany) was used for 3D reconstruction and analysis of the calibrated Micro-CT images.

Electrical measurements. Yield of injection. The yield of working devices after injection was determined by measuring the impedance of passive metal electrodes and conductance of nanowire devices before and after injection as follows: (1) As-made 2D mesh electronics were partially immersed in etchant solution as described above to release only the I/O region of mesh electronics and then mesh electronics was transferred to DI water and then dried in ethanol, while the released I/O region was unfolded on the substrate. (2) Next, the remaining nickel layer was etched and the sample transferred to DI water and dried in ethanol such that the device region was unfolded on the substrate. This two-step etching process allows the mesh electronics to fully unfold on the substrate in a manner that it can be subsequently re-suspended for injection. (3) Mesh electronics were modified by PDL as described above. (4a) For passive electrodes, the impedance (Z₀) at 1 kHz, and impedancefrequency (Z-f) data were recorded in 1× PBS using an Agilent B1500A semiconductor device parameter analyzer (Agilent Technologies Inc., Santa Clara, Calif.) with B1520A-FG multifrequency capacitance measurement unit (Agilent Technologies Inc., Santa Clara, Calif.). Electrodes with impedance at 1 kHz below 1.5 megohm were taken as suitable passive metal electrodes with total number, N0. (4b) For nanowire devices, the conductance (G₀) for each device was measured using a probe station (Lake Shore Cryotronics, Inc., Westerville, Ohio). Current-voltage (I-V) data were recorded using an Agilent 4156C semiconductor parameter analyzer (Agilent Technologies Inc., Santa Clara, Calif.) with contacts to device through probe station. Devices with conductance above 100 nS were taken as suitable nanowire devices with total number, N₀. (5) After impedance/conductance measurements, mesh electronics were immersed in DI water for 4 to 6 hours to suspend them, (6) mesh samples were transferred by glass pipette to PDL aqueous solution for surface modification as described above, and then (7) loaded into syringes fitted with ID needles from 100 to 600 micrometer and into a chamber with I/O unfolded on a substrate adjacent to the chamber. (8) Ethanol was used to rinse and dry the I/O. (9a) The impedance (Z₁) of the passive electrodes was measured as in step 4a, and the total number of electrodes meeting above criteria, N₁, post-injection was recorded. Yield and impedance changes in FIG. 16H were calculated as N₁/N₀ and (Z₁−Z₀)/Z₀, respectively. (9b) The conductance (G₁) of nanowire devices was measured again, and the total number, N₁, meeting the above criteria (step 4b above) was determined. Yield and conductance changes in FIG. 16I were calculated as N₁/N₀ and (G₁−G₀)/G₀, respectively. All measurements have been repeated for 16 different devices.

Test of ACF bonding. The connection resistance of ACF was measured to investigate the influence of bonding on electrical properties of devices. The conductance of each device (connected metal wires) was measured by probe station as R₀ and R₁ before and after ACF bonding, respectively. The connection resistance for each I/O pad (100 micrometer diameter) was calculated as (R1−R₀)/2. The calculated connection resistance after ACF bonding with commercial (ca. 21.2 ohm) and homemade (ca. 33.7 ohm) instruments, was <0.05% of the typical nanowire resistance and <0.01% of the typical metal electrode impedance at 1 kHz. The insulation resistance between I/O pads without circuits was over 10 gigaohm. These measurements and analyses demonstrate that ACF bonding had little influence on electrical properties of injectable mesh electronics, which ensured reliable measurements with injectable mesh electronics devices in the applications described above.

Piezoresistance measurements. The piezoresistance response of strained nanowire devices was measured as conductance change of device subject to the deformation of PDMS structure. In brief, the I/O pads were bonded to a flexible cable as described above, and connected to a multi-channel current/voltage preamplifier (Model 1211, DL Instruments, Brooktondale, N.Y.), filtered with a 3 kHz low pass filter (CyberAmp 380, Molecular Devices, Sunnyvale, Calif.), and digitized at a 1 kHz sampling rate (AxonDigi1440A, Molecular Devices, Sunnyvale, Calif.), with a 100 mV DC source bias voltage. Pressure was applied along z-axis for 20 sec using a homemade linear translation stage.

SU-8 passivation characterization. The effectiveness of SU-8 passivation was characterized following immersion in Neurobasal™ medium at 37° C. for 6 weeks using impedance-frequency (Z-f) measurement. A PDMS chamber 2 mm in longitudinal direction and 5 mm in transverse direction was positioned over the interconnect lines (without exposing the sensor electrodes), filled with 1× PBS solution, and then Z-f data were recorded using an Agilent B1500A semiconductor device parameter analyzer with B1520A-FG multi-frequency capacitance measurement unit. Significantly, impedance measurements from 1 to 10 kHz for 16 different SU-8 passivated metal interconnect lines showed average values above 10 gigaohm. The large impedance demonstrates that there is no obvious leakage through the thin SU-8 polymer passivation. In addition, the impedance at 1 kHz of the SU-8 passivated region, ˜30 G gigaohm is 10⁴-10⁵ larger than the typical values for the Pt-metal sensors.

Structure analysis and mechanical simulations. Number of rolls of mesh electronics inside glass needles. The mesh electronics rolls up in a scroll-like structure when injected through a glass needle. Theoretically, the number of circumferential rolls, Nᵣₒₗₗₛ can be calculated by dividing the total width, W, of the mesh with the perimeter of the tube, (pi)D, with, D, the tube ID, as Nᵣₒₗₗₛ=W/(pi)D with values of 3.5, 6.3, and 10.5 for FIGS. 17C to 17E, (I), (II) and (III), respectively. Experimentally, the number of circumferential rolls was estimated from the cross sections of 3D reconstructed confocal images as follows: First, the number of longitudinal ribbon (LR) features, K_{LR}, was counted in images of the scroll structure. Second, the number of LRs from a half circumference roll can be estimated as n_{LR}=(pi)D/2s, where s is the distance between LRs. Finally, the total number of circumference rolls is Nᵣₒₗₗₛ=2sK_{LR}/(pi)D, Using this method, the numbers of circumference rolls in FIGS. 17C to 17E were 3.4+/−0.2, 6.0 +/−0.4 and 9.5 +/−1.0 for (I), (II) and (III), respectively. The uncertainty arises from the identification of longitudinal elements from 8 random cross-sections for each case; small deviations from geometric analysis above may be arise in part from a failure to count some longitudinal elements due to low fluorescence intensity.

Mechanical simulation. Bending stiffness simulation. The bending stiffness of the mesh electronics with different structures was estimated by finite element software ABAQUS. A unit cell is used for the simulation, where the tilt angle alpha is defined in FIG. 16D and mesh electronics are modeled with shell elements. A homogeneous single shell section with 700 nm thick SU-8 is assigned to the transverse ribbons; a composite section with three layers of 350 nm thick SU-8, 100 nm thick gold and another 350 nm thick SU-8 is assigned to the longitudinal ribbons. Both SU-8 and gold are modeled as linear elastic materials, with Young's modulus 2 Gpa and 79 GPa respectively. To calculate the longitudinal and transverse bending stiffnesses, a fixed boundary condition is set at one of the ends parallel with the bending direction, and a small vertical displacement, d, is added at the other end. The external work, W, to bend the device is calculated. The effective bending stiffness of the device is defined as the stiffness required of a homogenous beam to achieve the same external work W under the displacement d. Therefore, the effective bending stiffness per width of the device can be estimated as:

$$D = \frac{2 {Wl}^{3}}{3 {d}^{2} b}$$

with b the width of the unit cell parallel with the bending direction, and l the length of the unit cell perpendicular to the bending direction.) .

Effective bending stiffnesses of implantable probes. The effective bending stiffness per width of the three-layer longitudinal ribbon, D₁, (longitudinal ribbon) in the mesh can be estimated as:

$${D}_{1} = \frac{{E}_{s}}{{w}_{1}} ( \frac{{h}^{3} {w}_{1}}{12} - \frac{{h}_{m}^{3} {w}_{m}}{12} ) + \frac{{E}_{m}}{{w}_{1}} \frac{{h}_{m}^{3} {w}_{m}}{12}$$

where Eₛ is Young's modulus of SU-8, Eₘ is Young's modulus of gold, h is the total thickness of ribbon, hₘ is the thickness of metal, w₁ is the total width of ribbon and wₘ is the width of metal. When Eₛ=2 GPa, Eₘ=79 GPa, h=800 nm, hₘ=100 nm, w₁=20 micrometer, wₘ=10 micrometer, D₁=0.086 nN m.

The effective bending stiffness per width of standard silicon probes, D₂, can be estimated as:

$${D}_{2} = {E}_{silicon} \frac{{h}_{silicon}^{3}}{12}$$

where E_{silicon} is the Young's modulus of silicon, h_{silicon} is the thickness of the probe. When E_{silicon}=165 GPa, h_{silicon}=15 micrometers, D₂=4.6×10₅ nN m .
The effective bending stiffness per width of ultra-small carbon electrodes, D₃, can be estimated as:

$${D}_{3} = {E}_{carbon} \frac{π {d}^{3}}{64}$$

where E_{carbon} is the Young's modulus of carbon fiber, d is the diameter of carbon fiber probe. When E_{carbon}=234 GPa, d=7 micrometer, D₃=3.9×10⁴ nN m.

The effective bending stiffness per width of planar shape probe, D₄, can be estimated as:

$${D}_{4} = {E}_{s} \frac{{h}_{s}^{3}}{12}$$

where Eₛ is the Young's modulus of polyimide, hₛ is the thickness of probe. When Eₛ=2-2.73 GPa, hₛ=10-20 micrometer, D₄=0.16-1.3×10⁴ nN m.

Simulation of mesh electronics strain. The data in FIGS. 16 and 17 show that mesh electronics can be injected in a rolled-up geometry through needles to 95 mm ID without breaking. The importance of the rolled up geometry during injection was quantified by using simulations to estimate the strain distribution versus needle ID the rolled-up geometry. The simulation treats a unit cell of the mesh bent with a radius of curvature, R, where a fixed boundary condition sets the strain of one longitudinal ribbon at zero and the maximal principal strain, epsilon-m, value then occurs at the junction between the transverse and second longitudinal element of the unit cell. This strain value represents an upper limit given that other edge of the unit cell was set to zero for the simulation. The plot of this upper limit strain value versus 1/R shows that strain increases linearly. The upper limit strain values extrapolated for a 100 micrometer ID needle for these two mesh structures, ca. 1.0%, are both smaller than the fracture strain, 5%, reported for a 20 micrometer thick SU-8 beam. In addition, the stress intensity factor, K, for a thin film under pure bending exhibits a square root dependence on thickness, K˜Eε\sqrt{h}, where E is the Young's modulus of the material, epsilon is the strain and h is the thickness of ribbon. The epsilon reaches the fracture strain of ribbon, epsilon-c, when K reaches the toughness of the material K_{c}. Since the thickness of SU-8 in the mesh structures is 700 nm (vs. 20 micrometers) the fracture strain of ribbon can be expected to be larger than 5%.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
passing a device comprising one or more nanoscale wires through an opening of a tube.

2-108. (canceled)
